(19) United States
(12) Patent Application Publication
(10) Pub. No.: US 2002/0091148 A1
(43) Pub. Date: Jul. 11, 2002

(54) 3-substituted indole antiproliferative angiogenesis inhibitors

(76) Inventors: Nwe Y. BaMaung, Niles, IL (US); Richard A. Craig, Racine, WI (US); Megumi Kawai, Libertyville, IL (US); Jieyi Wang, Lake Bluff, IL (US); Yujia Dai, Gurnee, IL (US); Yan Guo, Gurnee, IL (US); George Sheppard, Wilmette, IL (US); Mary K. Verzal, Kenosha, WI (US); Anil Vasudevan, Gurnee, IL (US); Michael Michaelides, Libertyville, IL (US)

Correspondence Address: Steven F. Weinstock, Abbott Laboratories, Department 377 / AP6D-2, 100 Abbott Park Road, Abbott Park, IL, 60064-6050, US

(21) Appl. No.: 09/952,603

(22) Filed: Sep. 14, 2001

Related U.S. Application Data
(60) Provisional application No. 60/233,390, filed on Sep. 15, 2000.

(51) Int. Cl. A61K 31/404

(52) U.S. Cl. 514/414; 514/419

(57) ABSTRACT

3-Substituted indole carbohydrazides having the formula

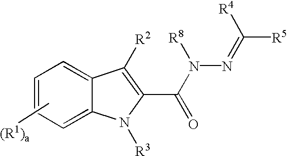

are useful for inhibiting angiogenesis and cell proliferation. Also disclosed are compositions which inhibit angiogenesis and cell proliferation and methods of inhibiting angiogenesis and cancer in a mammal.

### CROSS-REFERENCE TO RELATED APPLICATIONS

This application claims priority to U.S. Provisional Patent Application Ser. No. 60/233,390, filed on Sep. 15, 2000, which is hereby incorporated by reference.

### TECHNICAL FIELD

The present invention relates to 3-substituted indole carbohydrazides which are useful for inhibiting angiogenesis and cell proliferation, methods of making the compounds, compositions containing the compounds, and methods of treatment using the compounds.

### BACKGROUND OF THE INVENTION

Angiogenesis, the process by which new blood vessels are formed, is essential for normal body activities including reproduction, development, and wound repair. Although the process is not completely understood, it is believed to involve a complex interplay of molecules which regulate the growth of endothelial cells (the primary cells of capillary blood vessels). Under normal conditions, these molecules appear to maintain the microvasculature in a quiescent state (i.e., one of no capillary growth) for prolonged periods which may last for weeks or, in some cases, decades. When necessary (such as during wound repair), these same cells can undergo short bursts of growth and rapid proliferation (J. Biol. Chem. 1992, 267, 10931-10934, and Science 1987, 235, 442-447).

While it is normally a regulated process, many diseases (characterized as angiogenic diseases) are driven by persistent, unregulated angiogenesis. Ocular neovascularization has been implicated as the most common cause of blindness and is responsible for approximately twenty different eye diseases. In certain existing conditions, such as arthritis, newly formed capillary blood vessels invade the joints and destroy cartilage. The growth and metastasis of solid tumors are also dependent on angiogenesis (Cancer Res. 1986, 46, 467-473, and J. Natl. Cancer Inst. 1989, 82, 4-6). It has been shown that solid tumors cannot grow beyond 1 to 2 cubic millimeters without inducing the formation of new blood vessels. Once these new blood vessels become embedded in the tumor, they provide a means for tumor cells to enter the circulation and metastasize to distant sites such as the liver, the lungs, or the bones (N. Engl. J. Med. 1991, 324, 1-8).

Several chemotherapeutics used in cancer therapy are anti-angiogenic due to their ability to inhibit endothelial cell proliferation. The potential of such drugs to inhibit angiogenesis could be the result of their ability to cause collateral-damaging effects on cycling endothelial cells found in newly-formed blood vessels, or inhibiting other vital endothelial cell functions (such as microtubule synthesis) necessary for angiogenesis.

Although agents which inhibit angiogenesis and microtubule polymerization have been the subject of current research, there is still a need for compounds with improved profiles of activity.

### SUMMARY OF THE INVENTION

In its principle embodiment the present invention provides a method of treating cancer in a mammal in need of such therapy comprising administering to the mammal a therapeutically acceptable amount of a compound of formula (I),

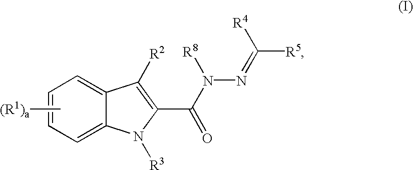

or a therapeutically acceptable salt thereof, wherein

a is 0, 1, 2, 3, or 4;

each R¹ is selected from the group consisting of alkoxy, amino, halo, hydroxy, and nitro;

R² is selected from the group consisting of alkenyl, alkoxy, alkyl, alkylsulfanyl, alkylsulfonyl, alkynyl, aminocarbonyl, Ar¹, arylalkyl, arylsulfanyl, arylsulfonyl, halo, and heterocycle;

R³ is selected from the group consisting of hydrogen, alkyl, and a nitrogen protecting group;

one of R⁴ and R⁵ is independently selected from the group consisting of alkyl, Ar², arylalkyl, cycloalkyl, (cycloalkyl)alkyl, heterocycle, and (heterocycle)alkyl; and the other is selected from the group consisting of hydrogen, and alkyl;

R⁸ is selected from the group consisting of hydrogen, and alkyl;

Ar¹ is an aryl group optionally substituted with one, two, three, four, or five substituents independently selected from the group consisting of alkenyl, alkoxy, alkoxyalkyl, alkyl, amino, cyano, cycloalkyl, (cycloalkyl)alkyl, halo, haloalkoxy, haloalkyl, hydroxy, and nitro; and

Ar² is an aryl group optionally substituted with one, two, three, four, or five substituents independently selected from the group consisting of alkenyl, alkoxy, alkoxyalkyl, alkoxycarbonyl, alkyl, alkylcarbonyl, alkylcarbonyloxy, alkylsulfanyl, alkylsulfonyl, amino, aminocarbonyl, aminosulfonyl, aminosulfonyloxy, cyano, cycloalkyl, (cycloalkyl)alkyl, formyl, halo, haloalkoxy, haloalkyl, heterocycle, hydroxy, hydroxyalkyl, and nitro.

In another embodiment the present invention provides a method of treating a mammal in need of anti-angiogenic therapy comprising administering to the mammal a therapeutically acceptable amount of a compound of formula (I), or a therapeutically acceptable salt thereof.

In another embodiment the present invention discloses a compound of formula (II),

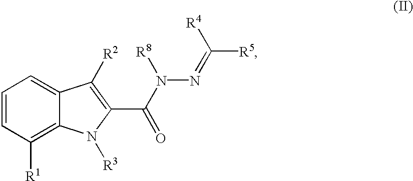

or a therapeutically acceptable salt thereof, wherein

R¹ is selected from the group consisting of hydrogen, alkoxy, amino, halo, and hydroxy;

R² is selected from the group consisting of alkenyl, alkoxy, alkyl, alkylsulfanyl, alkylsulfonyl, alkynyl, aminocarbonyl, Ar³, arylalkyl, arylsulfanyl, arylsulfonyl, halo, and heterocycle;

R³ is selected from the group consisting of hydrogen, alkyl, and a nitrogen protecting group;

one of R⁴ and R⁵ is independently selected from the group consisting of alkyl, Ar⁴, arylalkyl, cycloalkyl, (cycloalkyl)alkyl, heterocycle, and (heterocycle)alkyl; and the other is selected from the group consisting of hydrogen, and alkyl;

R⁸ is selected from the group consisting of hydrogen and alkyl;

Ar³ is an aryl group optionally substituted with one, two, three, four, or five substituents independently selected from the group consisting of alkenyl, alkoxy, alkoxyalkyl, alkyl, amino, cyano, cycloalkyl, (cycloalkyl)alkyl, halo, haloalkoxy, haloalkyl, hydroxy, and nitro; and

Ar⁴ is an aryl group optionally substituted with one, two, three, four, or five substituents independently selected from the group consisting of alkoxy, alkoxycarbonyl, alkyl, alkylsulfonyl, aminocarbonyl, aminosulfonyl, aminosulfonyloxy, cyano, halo, haloalkoxy, heterocycle, and hydroxy; with the proviso that when Ar³ is unsubstituted, Ar⁴ is substituted.

In another embodiment the present invention provides a pharmaceutical composition comprising a compound of formula (II), or a therapeutically acceptable salt thereof, in combination with a therapeutically acceptable carrier.

### DETAILED DESCRIPTION OF THE INVENTION

Compounds of the present invention comprise 3-substituted indole carbohydrazides which are useful for the treatment of diseases caused or exacerbated by angiogenesis and/or cell proliferation.

As used in the present specification the following terms have the meanings indicated:

The term “alkenyl,” as used herein, represents a straight or branched chain group of one to six carbon atoms containing at least one carbon-carbon double bond.

The term “alkoxy,” as used herein, represents an alkyl group attached to the parent molecular moiety through an oxygen atom.

The term “alkoxyalkyl,” as used herein, represents an alkoxy group attached to the parent molecular moiety through an alkyl group.

The term “alkoxycarbonyl,” as used herein, represents an alkoxy group attached to the parent molecular moiety through a carbonyl group.

The term “alkyl,” as used herein, represents a group of one to six carbon atoms derived from a straight or branched chain saturated hydrocarbon.

The term “alkylcarbonyl,” as used herein, represents an alkyl group attached to the parent molecular moiety through a carbonyl group.

The term “alkylcarbonylalkyl,” as used herein, represents an alkylcarbonyl group attached to the parent molecular moiety through an oxygen atom.

The term “alkylcarbonyloxy,” as used herein, represents an alkylcarbonyl group attached to the parent molecular moiety through an oxygen atom.

The term “alkylcarbonyloxyalkyl,” as used herein, represents an alkylcarbonyloxy group attached to the parent molecular moiety through an alkyl group.

The term “alkylsulfanyl,” as used herein, represents an alkyl group attached to the parent molecular moiety through a sulfur atom.

The term “alkylsulfonyl,” as used herein, represents an alkyl group attached to the parent molecular moiety through a sulfonyl group.

The term “alkynyl,” as used herein, represents a straight or branched chain group of one to six carbon atoms containing at least one carbon-carbon triple bond.

The term “amido,” as used herein, represents an amino group attached to the parent molecular moiety through a carbonyl group.

The term “amino,” as used herein, represents —NR⁶R⁷, wherein R⁶ and R⁷ are independently selected from the group consisting of hydrogen, alkenyl, alkyl, alkylcarbonyl, aryl, cycloalkyl, (cycloalkyl)alkyl, and a nitrogen protecting group.

The term “aminocarbonyl,” as used herein, represents an amino group attached to the parent molecular moiety through a carbonyl group.

The term “aminosulfonyl,” as used herein, represents an amino group attached to the parent molecular moiety through a sulfonyl group.

The term “aminosulfonyloxy,” as used herein, represents an aminosulfonyl group attached to the parent molecular moiety through an oxygen atom.

The term “antiproliferative therapy,” as used herein, represents the treatment of conditions which are caused or exacerbated by angiogenesis and/or cell mitosis (e.g., cancer).

The term “aryl,” as used herein, represents dihydronaphthyl, indanyl, indenyl, naphthyl, phenyl, and tetrahydronaphthyl. Aryl groups having an unsaturated or partially saturated ring fused to an aromatic ring can be attached through the saturated or the unsaturated part of the group.

The term “arylalkyl,” as used herein, represents an aryl group attached to the parent molecular moiety through an alkyl group.

The term “arylsulfanyl,” as used herein, represents an aryl group attached to the parent molecular moiety through a sulfur atom.

The term “arylsulfonyl,” as used herein, represents an aryl group attached to the parent molecular moiety through a sulfonyl group.

The term “carbonyl,” as used herein, represents —C(O)—.

The term “cyano,” as used herein, represents —CN.

The term “cycloalkyl,” as used herein, represents a saturated cyclic, bicyclic, or tricyclic hydrocarbon ring system having three to twelve carbon atoms. Examples of cycloalkyl groups include cyclopropyl, cyclopentyl, bicyclo(3,1,1)heptyl, adamantyl, and the like.

The term “(cycloalkyl)alkyl,” as used herein, represents a cycloalkyl group attached to the parent molecular moiety through an alkyl group.

The term “formyl,” as used herein, represents —CHO.

The term “halo,” as used herein, represents F, Cl, Br, or I.

The term “haloalkoxy,” as used herein, represents a haloalkyl group attached to the parent molecular moiety through an oxygen atom.

The term “haloalkyl,” as used herein, represents an alkyl group substituted by one, two, three, or four halogen atoms.

The term “heterocycle,” as used herein, represents a five-, six-, or seven-membered ring containing one, two, or three heteroatoms independently selected from the group consisting of nitrogen, oxygen, and sulfur. The five-membered ring has zero to two double bonds and the six- and seven-membered rings have zero to three double bonds. The term “heterocycle” also includes bicyclic groups in which the heterocycle ring is fused to an aryl group. The heterocycle groups of this invention can be attached through a carbon atom or a nitrogen atom in the ring. The heterocycle groups of this invention can also be optionally substituted with one, two, three, or four substituents independently selected from the group consisting of alkenyl, alkoxy, alkoxyalkyl, alkoxycarbonyl, alkyl, alkylcarbonyl, alkylcarbonyloxyalkyl, alkylsulfanyl, alkylsulfonyl, amido, amino, aminosulfonyl, aryl, carbonyloxy, carbonyloxyalkyl, cyano, cycloalkyl, (cycloalkyl)alkyl, formyl, halo, haloalkoxy, haloalkyl, hydroxy, hydroxyalkyl, and nitro, wherein the aryl can be further optionally substituted with one, two, or three substituents independently selected from the group consisting of alkoxy, alkyl, halo, haloalkoxy, haloalkyl, hydroxy, and nitro.

The term “(heterocycle)alkyl,” as used herein, represents a heterocycle group attached to the parent molecular moiety through an alkyl group.

The term “hydroxy,” as used herein, represents —OH.

The term “hydroxyalkyl,” as used herein, represents a hydroxy group attached to the parent molecular moiety through an alkyl group.

The term “nitro,” as used herein, represents —NO₂.

The term “nitrogen protecting group,” as used herein, represents groups intended to protect an amino group against undesirable reactions during synthetic procedures. Common N-protecting groups comprise acyl groups such as acetyl, benzoyl, 2-bromoacetyl, 4-bromobenzoyl, tert-butylacetyl, carboxaldehyde, 2-chloroacetyl, 4-chlorobenzoyl, α-chlorobutyryl, 4-nitrobenzoyl, o-nitrophenoxyacetyl, phthalyl, pivaloyl, propionyl, trichloroacetyl, and trifluoroacetyl; sulfonyl groups such as benzenesulfonyl, and p-toluenesulfonyl; carbamate forming groups such as benzyloxycarbonyl, benzyloxycarbonyl (Cbz), tert-butyloxycarbonyl (Boc), p-chlorobenzyloxycarbonyl, p-methoxybenzyloxycarbonyl, and the like.

The term “prodrug,” refers to compounds which are rapidly transformed in vivo to parent compounds of formula (I) for example, by hydrolysis in blood.

The term “sulfonyl,” as used herein, represents —SO₂—.

The present compounds can also exist as therapeutically acceptable prodrugs. The term “therapeutically acceptable prodrug,” refers to those prodrugs or zwitterions which are suitable for use in contact with the tissues of patients without undue toxicity, irritation, and allergic response, are commensurate with a reasonable benefit/risk ratio, and are effective for their intended use.

The term “therapeutically acceptable salt,” as use herein, represents those salts which are, within the scope of sound medical judgment, suitable for use in contact with the tissues of humans and lower animals without undue toxicity, irritation, allergic response and the like and are commensurate with a reasonable benefit/risk ratio. The salts can be prepared in situ during the final isolation and purification of the compounds of the present invention or separately by reacting the free base group with a suitable organic acid. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, trifluoroacetate, undecanoate, valerate salts, and the like. Representative alkali or alkaline earth metal salts include calcium, lithium, magnesium, potassium, sodium, and the like, as well as non-toxic ammonium, quaternary ammonium, and amine cations, including, but not limited to, ammonium, dimethylamine, ethylamine, methylamine, tetraethylammonium, tetramethylammonium, triethylamine, trimethylamine, and the like.

Because carbon-carbon double bonds exist in the present compounds, the invention contemplates various geometric isomers and mixtures thereof resulting from the arrangement of substituents around these carbon-carbon double bonds. It should be understood that the invention encompasses both isomeric forms, or mixtures thereof, which possess the ability to inhibit angiogenesis and/or cell proliferation. These substituents are designated as being in the E or Z configuration wherein the term “E” represents higher order substituents on opposite sides of the carbon-carbon double bond, and the term “Z” represents higher order substituents on the same side of the carbon-carbon double bond.

In accordance with methods of treatment and pharmaceutical compositions of the invention, the compounds can be administered alone or in combination with other anti-angiogenic and/or antimitotic agents. When using the compounds, the specific therapeutically effective dose level for any particular patient will depend upon factors such as the disorder being treated and the severity of the disorder; the activity of the particular compound used; the specific composition employed; the age, body weight, general health, sex, and diet of the patient; the time of administration; the route of administration; the rate of excretion of the compound employed; the duration of treatment; and drugs used in combination with or coincidently with the compound used. The compounds can be administered orally, parenterally, osmotically (nasal sprays), rectally, vaginally, or topically in unit dosage formulations containing carriers, adjuvants, diluents, vehicles, or combinations thereof. The term “parenteral” includes infusion as well as subcutaneous, intravenous, intramuscular, and intrasternal injection.

Parenterally administered aqueous or oleaginous suspensions of the compounds can be formulated with dispersing, wetting, or suspending agents. The injectable preparation can also be an injectable solution or suspension in a diluent or solvent. Among the acceptable diluents or solvents employed are water, saline, Ringer's solution, buffers, monoglycerides, diglycerides, fatty acids such as oleic acid, and fixed oils such as monoglycerides or diglycerides.

The inhibitory effect of parenterally administered compounds can be prolonged by slowing their absorption. One way to slow the absorption of a particular compound is administering injectable depot forms comprising suspensions of crystalline, amorphous, or otherwise water-insoluble forms of the compound. The rate of absorption of the compound is dependent on its rate of dissolution which is, in turn, dependent on its physical state. Another way to slow absorption of a particular compound is administering injectable depot forms comprising the compound as an oleaginous solution or suspension. Yet another way to slow absorption of a particular compound is administering injectable depot forms comprising microcapsule matrices of the compound trapped within liposomes, microemulsions, or biodegradable polymers such as polylactide-polyglycolide, polyorthoesters or polyanhydrides. Depending on the ratio of drug to polymer and the composition of the polymer, the rate of drug release can be controlled.

Transdermal patches can also provide controlled delivery of the compounds. The rate of absorption can be slowed by using rate controlling membranes or by trapping the compound within a polymer matrix or gel. Conversely, absorption enhancers can be used to increase absorption.

Solid dosage forms for oral administration include capsules, tablets, pills, powders, and granules. In these solid dosage forms, the active compound can optionally comprise diluents such as sucrose, lactose, starch, talc, silicic acid, aluminum hydroxide, calcium silicates, polyamide powder, tableting lubricants, and tableting aids such as magnesium stearate or microcrystalline cellulose. Capsules, tablets and pills can also comprise buffering agents, and tablets and pills can be prepared with enteric coatings or other release-controlling coatings. Powders and sprays can also contain excipients such as talc, silicic acid, aluminum hydroxide, calcium silicate, polyamide powder, or mixtures thereof. Sprays can additionally contain customary propellants such as chlorofluorohydrocarbons or substitutes therefor.

Liquid dosage forms for oral administration include emulsions, microemulsions, solutions, suspensions, syrups, and elixirs comprising inert diluents such as water. These compositions can also comprise adjuvants such as wetting, emulsifying, suspending, sweetening, flavoring, and perfuming agents.

Topical dosage forms include ointments, pastes, creams, lotions, gels, powders, solutions, sprays, inhalants, and transdermal patches. The compound is mixed under sterile conditions with a carrier and any needed preservatives or buffers. These dosage forms can also include excipients such as animal and vegetable fats, oils, waxes, paraffins, starch, tragacanth, cellulose derivatives, polyethylene glycols, silicones, bentonites, silicic acid, talc and zinc oxide, or mixtures thereof. Suppositories for rectal or vaginal administration can be prepared by mixing the compounds with a suitable nonirritating excipient such as cocoa butter or polyethylene glycol, each of which is solid at ordinary temperature but fluid in the rectum or vagina. Ophthalmic formulations comprising eye drops, eye ointments, powders, and solutions are also contemplated as being within the scope of this invention.

The total daily dose of the compounds administered to a host in single or divided doses can be in amounts from about 0.1 to about 200 mg/kg body weight or preferably from about 0.25 to about 100 mg/kg body weight. Single dose compositions can contain these amounts or submultiples thereof to make up the daily dose.

Preferred embodiments of the present invention include, but are not limited to: compounds of formula (I) wherein one of R⁴ and R⁵ is Ar² or heterocycle and the other is hydrogen; compounds of formula (I) wherein one R² is Ar¹ or alkyl; compounds of formula (II), wherein one of R⁴ and R⁵ is Ar⁴ or heterocycle and the other is hydrogen; and compounds of formula (II), wherein R² is Ar³ or alkyl.

Determination of Biological Activity

Human neonatal dermal microvascular endothelial cells (HMVEC) and their recommended culture media (EGM2) were purchased from Clonetics (San Diego, Calif.). Cells were grown in EGM2 with 5% FBS of instructions provided by Clonetics. Cell proliferation assays were performed in 96-well plates using cells between passages 6 and 12. Cells were seeded at 3000-5000 cells/well in 180 μL/well EGM2 with 5% FBS and were allowed to attach for 4 hours at 5% CO₂ in a 37° C. incubator. All compounds were dissolved in DMSO at 10 mM and were diluted with 50 mM Hepes buffer (pH 7.4) in 100 mM NaCl to final concentrations of 0.01 μM, 0.1 μM, 1 μM, 10 μM, 100 μM, and 1000 μM. Each well of the culture plate was treated with 20 μL of the diluents resulting in final concentrations of 0.001 μM, 0.01 μM, 0.1 μM, 1 μM, 10 μM, and 100 μM. The cells were returned to 5% CO₂ in a 37° C. incubator for 3 days. Live cells were quantitated with MTS reagents (Promega, Madison Wis.). IC₅₀ values were calculated from dose response curves. Compounds of the present invention had IC₅₀ values between 9 nm and 60 μM with a preferred range of 0.1 μM-0.5 μM and a most preferred range of 9 nM-50 nM. As it has been shown that SU5416, a small molecule which inhibits endothelial cell proliferation, has good in vivo activity against certain tumor models, it can therefore be extrapolated that the compounds of the invention, including but not limited to those specified in the examples, are useful for the treatment of diseases caused or exacerbated by angiogenesis (Adv. Cancer Res. 2000, 79, 1-38).

The cell morphology change upon treatment with these compounds indicated cellular microtubules might be a target. Cellular microtubule staining studies confirmed this hypothesis. In vitro tubulin polymerization and colchicine/vinblastine binding assays further showed these compounds bind to tubulin and can compete off both colchicine and vinblastine's binding to tubulin.

Microtubule polymerization was carried out with the CytoDYNAMIX Screen 1 kit from Cytoskeleton (Denver, Colo.) following its instruction manual. Briefly, compounds (1 μM-1000 μM final concentration) were incubated with 30 μM tubulin at 37° C., and the micortubule polymerization was followed by recording optical density at 340 nm. The IC50s of these compounds for microtubule polymerization were from 1 μM to 1000 μM.

Tubulin colchicine or vinblastine binding site competitive assays were performed with assay kits purchased from Cytoskeleton (Denver, Colo.) following their instruction manuals. Briefly, compounds (1 μM-1000 μM) were incubated with tubulin (10 μM) and fluorescent colchicine (5 μM) or fluorescent vinblastine (5 μM), and tubulin bound colchicines or vinblastine were quantitated by fluorescent spectrophotometer after size exclusion chromatography. The compounds of the present invention were active in competing off both colchicine and vinblastine binding to tubulin. The IC50s of these compounds to compete colchicine or vinblastine binding to tubulin ranged from 1 μM to 1000 μM.

As angiogenesis and cell proliferation inhibitors, these compounds are useful in the treatment of both primary and metastatic solid tumors and carcinomas of, for example, the breast, colon, rectum, lung, oropharynx, hypopharynx, esophagus, stomach, pancreas, liver, gallbladder, bile ducts, small intestine, urinary tract including kidney, bladder and urothelium, female genital tract including cervix, uterus, ovaries, choriocarcinoma, and gestational trophoblastic disease, male genital tract including prostate, seminal vesicles, testes, and germ cell tumors, endocrine glands including thyroid, adrenal, and pituitary, skin including hemangiomas, melanomas, sarcomas arising from bone or soft tissues including Kaposi's sarcoma, tumors of the brain, nerves, and eyes, meninges including astrocytomas, gliomas, glioblastomas, retinoblastomas, neuromas, neuroblastomas, Schwannomas and meningiomas, solid tumors arising from hematopoietic malignancies including leukemias and chloromas, plasmacytomas, plaques, tumors of mycosis fungoides, cutaneous T-cell lymphoma/leukemia, lymphomas including Hodgkin's and non-Hodgkin's lymphomas, prophylaxis of autoimmune diseases including rheumatoid, immune and degenerative arthritis, ocular diseases including diabetic retinopathy, retinopathy of prematurity, corneal graft rejection, retrolental fibroplasia, neovascular glaucoma, rubeosis, retinal neovascularization due to macular degeneration, hypoxia, abnormal neovascularization conditions of the eye, skin diseases including psoriasis, blood vessel diseases including hemagiomas and capillary proliferation within atherosclerotic plaques, Osler-Webber Syndrome, myocardial angiogenesis, plaque neovascularization, telangiectasia, hemophiliac joints, angiofibroma, and wound granulation.

Synthetic Methods

Abbreviations which have been used in the descriptions of the scheme and the examples that follow are: THF for tetrahydrofuran, and DMSO for dimethylsulfoxide.

The compounds and processes of the present invention will be better understood in connection with the following synthetic schemes which illustrate methods by which the compounds of the invention can be prepared. The compounds defined above can be prepared by a variety of synthetic routes. Representative procedures are shown in Scheme 1. Starting materials can be obtained from commercial sources or prepared by well-established literature methods known to those of ordinary skill in the art. The groups R¹, R², R³, R⁴, and R⁵ are defined above. It will be readily apparent to one of ordinary skill in the art that the compounds defined above can be synthesized by substitution of the appropriate reactants and agents in the syntheses shown below.

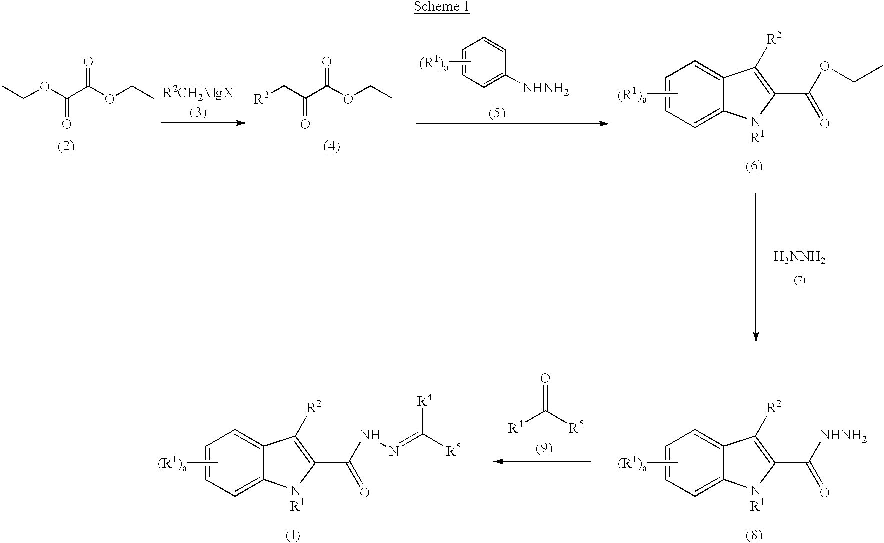

As shown in Scheme 1, compounds of formula (2) can be reacted with compounds of formula (3) (X is Cl, Br, or I) to provide compounds of formula (4). Examples of solvents used in these reactions include diethyl ether, THF, and methyl tert-butyl ether. The reaction is conducted at about −100° C. to 0° C. and depends on the solvent chosen. Reaction times are typically about 20 to about 60 minutes.

Compounds of formula (4) can be reacted with compounds of formula (5) in the presence of acid to provide compounds of formula (6) (R¹ is H). Representative acids include sulfuric acid, hydrochloric acid, and acetic acid. Examples of solvents used in these reactions include ethanol, isopropanol, and methanol. The reaction is conducted at about 60° C. to about 130° C. Reaction times are typically about 30 minutes to about 2 hours.

Conversion of compound of formula (6) (R¹ is H) to compounds of formula (8) (R¹ is H) can be accomplished by treatment with hydrazine (7) or hydrazine hydrate. Examples of solvents used in these reactions include ethanol, isopropanol, and methanol. The reaction is conducted at about 60° C. to about 95° C. and depends on the solvent chosen. Reaction times are typically about 12 to about 24 hours. Compounds of formula (8) (R¹ is H) can be condensed with compounds of formula (9) to provide compounds of formula (I) (R¹ is H). Examples of solvents used in these reactions include ethanol, methanol, and isopropanol. The reaction is conducted at about 60° C. to about 95° C. and depends on the solvent chosen. Reaction times are typically about 12 to about 24 hours.

Compounds of formula (I) (R¹ is H) can be intraconverted to compounds of formula (I) (R¹ is alkyl or a nitrogen protecting group) by methods known to those of ordinary skill in the art.

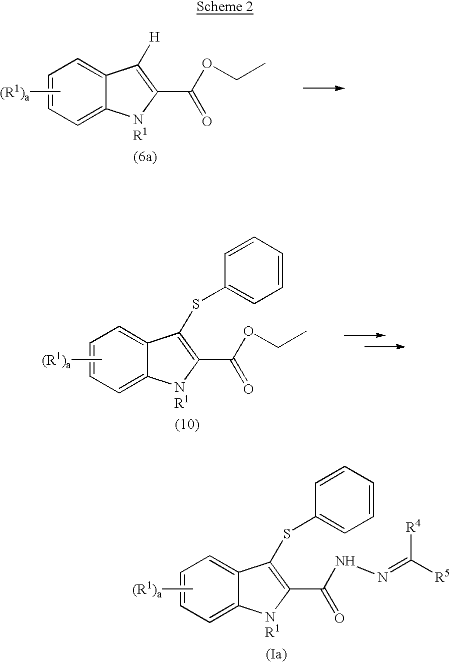

Scheme 2 shows the synthesis of compounds of formula (Ia). Compounds of formula (6a) can be converted to compounds of formula (7) by treatment with a base such as sodium hydride or sodium hexamethyldisilazide, followed by treatment with diphenyl disulfide. Examples of solvents used in these reactions include N,N-dimethylformamide and N-methylpyrrolidinone. Compounds of formula (7) can be converted to compounds of formula (Ia) by the methods described in Scheme 1. Alternatively, compounds of formula (7) can be treated with an oxidizing agent such as mCPBA or KMnO₄ to provide the corresponding sulfone.

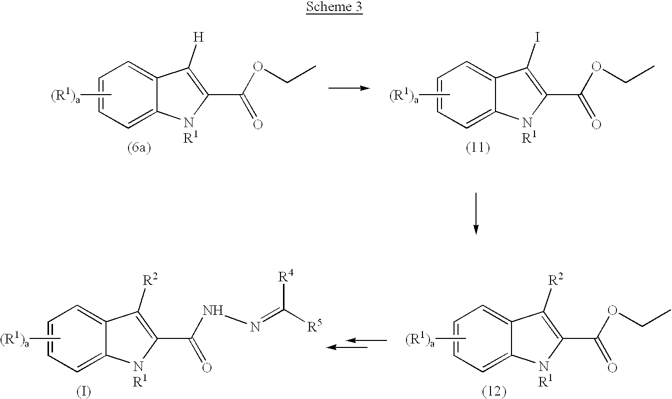

Compounds of formula (I) wherein R² is an aryl or heterocycle group can be prepared by the methods described in Scheme 3. Compounds of formula (6a) can be reacted with a strong base such as potassium hydroxide or sodium hydroxide in a solvent such as N,N-dimethylformamide or 1,2-dimethoxyethane, then treated with iodine to provide compounds of formula (11). Typical reaction temperatues are about 20° C. to about 35° C. These compounds can be converted to compounds of formula (12) by treatment with the appropriately substituted boronic acid or ester in the presence of a palladium catalyst and a base. Examples of palladium catalysts include palladium chloride, palladium dibenzylideneacetone, and palladium tetrakistriphenylphosphine. Representative bases include sodium carbonate and potassium carbonate. Typical solvents include 1,2-dimethoxyethane and N-methylpyrrolidinone and typical reaction temperatures are between about 70° C. and about 90° C.

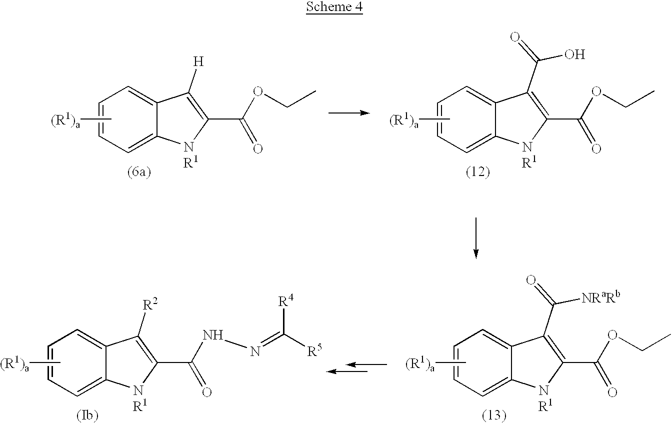

As shown in Scheme 4, compounds of formula (6a) can be converted to compounds of formula (12) by treatment with POCl₃ in a solvent such as ethanol followed by treatment with NaClO₂ in the presence of NaH₂PO₄ in a solvent such as t-butanol. Compounds of formula (12) can be converted to compounds of formula (13) by treatment with an appropriately substituted amine in the presence of hydroxybenzotriazole, 1-ethyl-3-(3-(dimethylamino)propyl)-carbodiimide hydrochloride and N-methylmorpholine in a solvent such as DMF. Compounds of formula (13) can be converted to compounds of formula (Ib) by the methods described in Scheme 1.

The present invention will now be described in connection with certain preferred embodiments which are not intended to limit its scope. On the contrary, the present invention covers all alternatives, modifications, and equivalents as can be included within the scope of the claims. Thus, the following examples, which include preferred embodiments, will illustrate the preferred practice of the present invention, it being understood that the examples are for the purposes of illustration of certain preferred embodiments and are presented to provide what is believed to be the most useful and readily understood description of its procedures and conceptual aspects.

### EXAMPLE 1

### N′-((4-methoxyphenyl)methylidene)-3-phenyl-1H-indole-2-carbohydrazide

### EXAMPLE 1A

### Ethyl 2-oxo-3-phenylpropanoate

A solution of diethyl oxalate (11.15 mL, 82.1 mmol) in diethyl ether (50 mL) at −78° C. was treated dropwise with 1M benzylmagnesium chloride in diethyl ether (82 mL, 82 mmol) while maintaining an internal temperature of −60° C. The mixture was stirred for 30 minutes and poured into a mixture of concentrated HCl (8 mL), ice (40 mL), and diethyl ether (50 mL). The organic phase was washed with water and brine, dried (MgSO₄), filtered, and concentrated to provide 15.5 g (98%) of the desired product of sufficient purity for subsequent use.

### EXAMPLE 1B

### Ethyl 3-phenyl-1H-indole-2-carboxylate

A mixture of Example 1A (7.81 g, 40.7 mmol) and phenylhydrazine (4.00 mL, 40.7 mmol) was treated with concentrated sulfuric acid (4 drops), heated to 120° C. for 15 minutes, cooled to room temperature, treated with ethanol (50 mL), treated with bubbling HCl gas for 2 minutes, and heated to reflux for 1 hour. The mixture was poured into water (100 mL) and extracted with diethyl ether. The combined extracts were washed with water and brine, dried (Na₂SO₄), filtered, and concentrated. The concentrate was recrystallized from ethanol to provide 3.43 g (32%) of the desired product.

### EXAMPLE 1C

### 3-phenyl-1H-indole-2-carbohydrazide

A solution of Example 1B (2.65 g, 10 mmol) in ethanol (20 mL) was treated with hydrazine hydrate (3.12 mL, 100 mmol), heated to reflux for 18 hours, cooled to room temperature, and filtered. The resulting solid was washed with ethanol and dried under vacuum to provide 1.86 g (74%) of the desired product of sufficient purity for subsequent use.

### EXAMPLE 1D

### N′-((4-methoxyphenyl)methylidene)-3-phenyl-1H-indole-2-carbohydrazide

A solution of Example 1C (1.76 g, 7.0 mmol) and p-anisaldehyde (894 mL, 7.35 mmol) in ethanol (120 mL) was refluxed for 18 hours, cooled to room temperature, and filtered. The resulting solid was washed with ethanol and dried under vacuum to provide 2.08 g (80%) of the desired product. MS (ESI(+)) m/e 370 (M+H)⁺.

### EXAMPLE 2

### N′-((4-bromophenyl)methylidene)-3-phenyl-1H-indole-2-carbohydrazide

A solution of Example 1C (1.76 g, 7.0 mmol) and 4-bromobenzaldehyde (1.36 g, 7.35 mmol) in ethanol (120 mL) was refluxed for 18 hours, cooled to room temperature, and filtered. The resulting solid was washed with ethanol and dried under vacuum to provide 2.38 g (81%) of the desired product. MS (ESI(+)) m/e 420 (M+H)⁺; ¹H NMR (300 MHz, DMSO-d₆) δ 7.09-7.16 (m, 1H), 7.26-7.57 (m, 9H), 7.58-7.72 (m, 3H), 8.05 (s, 1H).

### EXAMPLE 3

### 3-phenyl-N′-((4-(trifluoromethoxy)phenyl)methylidene)-1H-indole-2-carbohydrazide

The desired product was prepared by substituting 4-(trifluoromethoxy)benzaldehyde for 4-bromobenzaldehyde in Example 2, then purifying the resulting product by flash column chromatography on silica gel with 15% acetone/hexanes. MS (ESI(+)) m/e 424 (M+H)⁺; ¹H NMR (300 MHz, DMSO-d₆) δ 7.10-7.16 (m, 1H), 7.25-7.58 (m, 9H), 7.65 (d, 1H), 7.73-7.92 (br s, 2H), 8.12 (s, 1H).

### EXAMPLE 4

### N′-((4-(difluoromethoxy)phenyl)methylidene)-3-phenyl-1H-indole-2-carbohydrazide

The desired product was prepared by substituting 4-(difluoromethoxy)benzaldehyde for 4-bromobenzaldehye in Example 2, then purifying the resulting product by flash column chromatography on silica gel with 15% acetone/hexanes. MS (ESI(+)) m/e 406 (M+H)⁺; ¹H NMR (300 MHz, DMSO-d₆) δ 7.06-7.16 (m, 2H), 7.20-7.60 (m, 9H), 7.65 (d, 1H), 7.72-7.81 (br m, 1H), 8.07 (s, 1H).

### EXAMPLE 5

### N′-((3-bromophenyl)methylidene)-3-phenyl-1H-indole-2-carbohydrazide

The desired product was prepared by substituting 3-bromobenzaldehyde for 4-bromobenzaldehyde in Example 2, then purifying the resulting product by flash column chromatography on silica gel with 15% acetone/hexanes. MS (ESI(−)) m/e 418 (M−H)⁻; ¹H NMR (300 MHz, DMSO-d₆) δ 7.11-7.17 (m, 1H), 7.25-8.86 (m, 13H).

### EXAMPLE 6

### N′-((4-(dihydroxyamino)phenyl)methylidene)-3-phenyl-1H-indole-2-carbohydrazide

The desired product was prepared by substituting 4-nitrobenzaldehyde for 4-bromobenzaldehyde in Example 2. MS (ESI(+)) m/e 385 (M+H)⁺; ¹H NMR (300 MHz, DMSO-d₆) δ 7.11-7.16 (m, 1H), 7.26-7.34 (m, 1H), 7.36-7.57 (m, 6H), 7.66 (d, 1H), 7.81-8.33 (m, 5H).

### EXAMPLE 7

### 3-phenyl-N′-(phenylmethylidene)-1H-indole-2-carbohydrazide

The desired product was prepared by substituting benzaldehyde for 4-bromobenzaldehyde in Example 2. MS (ESI(+)) m/e 340 (M+H)⁺; ¹H NMR (300 MHz, DMSO-d₆) δ 7.09-7.15 (m, 1H), 7.26-7.58 (m, 10H), 7.62-7.75 (m, 3H), 8.07 (s, 1H).

### EXAMPLE 8

### N′-((3-cyanophenyl)methylidene)-3-phenyl-1H-indole-2-carbohydrazide

The desired product was prepared by substituting 3-cyanobenzaldehyde for 4-bromobenzaldehyde in Example 2. MS (ESI(+)) m/e 365 (M+H)⁺; ¹H NMR (300 MHz, DMSO-d₆) δ 7.11-7.16 (m, 1H), 7.26-7.33 (m, 1H), 7.37-8.21 (m, 11H).

### EXAMPLE 9

### N′-((4-methoxyphenyl)methylidene)-3-methyl-1H-indole-2-carbohydrazide

The desired product was prepared by substituting p-anisaldehyde and Example 61C for 4-bromobenzaldehyde and Example 1C, respectively, in Example 2. MS (ESI(+)) m/e 308 (M+H)⁺.

### EXAMPLE 10

### N′-((4-cyanophenyl)methylidene)-3-phenyl-1H-indole-2-carbohydrazide

The desired product was prepared by substituting 4-cyanobenzaldehyde for 4-bromobenzaldehyde in Example 2. MS (ESI(+)) m/e 365 (M+H)⁺, ¹H NMR (300 MHz, DMSO-d₆) δ 7.10-7.16 (m, 1H), 7.26-7.32 (m, 1H), 7.36-7.48 (br s, 2H), 7.48-7.56 (m, 2H), 7.96 (br s, 3H), 8.11 (br s, 1H).

### EXAMPLE 11

### N-(4-((2-((3-phenyl-1H-indol-2-yl)carbonyl)hydrazono)methyl)phenyl)acetamide

The desired product was prepared by substituting 4-acetamidobenzaldehyde for 4-bromobenzaldehyde in Example 2. MS (ESI(+)) m/e 397 (M+H)⁺; ¹H NMR (300 MHz, DMSO-d₆) δ 2.06 (s, 3H), 7.09-7.15 (m, 1H), 7.25-7.31 (m, 1H), 7.32-7.38 (br s, 1H), 7.40-7.70 (m, 9H), 8.00 (s, 1H).

### EXAMPLE 12

### N′-((4-(diethylamino)phenyl)methylidene)-3-phenyl-1H-indole-2-carbohydrazide

The desired product was prepared by substituting 4-diethylaminobenzaldehyde for 4-bromobenzaldehyde in Example 2. MS (ESI(+)) m/e 411 (M+H)⁺; ¹H NMR (300 MHz, DMSO-d₆) δ 1.10 (br t, 6H), 3.34-3.41 (br m, 4H), 6.52-6.73 (m, 2H), 7.08-7.15 (m, 1H), 7.25-7.29 (m, 1H), 7.30-7.38 (m, 1H), 7.40-7.57 (m, 6H), 7.62 (d, 1H), 7.87 (s, 1H).

### EXAMPLE 13

### N′-((4-isopropylphenyl)methylidene)-3-phenyl-1H-indole-2-carbohydrazide

The desired product was prepared by substituting 4-isopropylbenzaldehyde for 4-bromobenzaldehyde in Example 2. MS (ESI(+)) m/e 382 (M+H)⁺; ¹H NMR (300 MHz, DMSO-d₆) δ 1.20 (d, 6H), 2.92 (br m, 1H), 7.10-7.15 (m, 1H), 7.25-7.67 (m, 12H), 8.03 (s. 1H).

### EXAMPLE 14

### N′-((3-nitrophenyl)methylidene)-3-phenyl-1H-indole-2-carbohydrazide

The desired product was prepared by substituting 3-nitrobenzaldehyde for 4-bromobenzaldehyde in Example 2. MS (ESI(−)) m/e 383 (M−H)⁻; ¹H NMR (300 MHz, DMSO-d₆) δ 7.11-7.16 (m, 1H), 7.26-7.33 (t, 1H), 7.35-7.60 (m, 5H), 7.63-7.77 (m, 2H), 7.96-8.60 (br m, 3H).

### EXAMPLE 15

### 3-phenyl-N′-((4-(1-pyrrolidinyl)phenyl)methylidene)-1H-indole-2-carbohydrazide

The desired product was prepared by substituting 4-pyrrolidinobenzaldehyde for 4-bromobenzaldehyde in Example 2. MS (ESI(+)) m/e 409 (M+H)⁺; ¹H NMR (300 MHz, DMSO-d₆) δ 1.95 (br s, 4H), 6.53-6.63 (br m, 2H), 7.09-7.14 (m, 1H), 7.24-7.30 (m, 1H), 7.32-7.37 (br m., 1H), 7.40-7.58 (m, 7H), 7.63 (br d, 1H), 7.88 (s, 1H).

### EXAMPLE 16

### N′-((4-(methylsulfonyl)phenyl)methylidene)-3-phenyl-1H-indole-2-carbohydrazide

The desired product was prepared by substituting 4-methylsulfonylbenzaldehyde for 4-bromobenzaldehyde in Example 2. MS (ESI(−)) m/e 416 (M−H)⁻; ¹H NMR (300 MHz, DMSO-d₆) δ 3.23 (s, 3H), 7.11-7.16 (m, 1H), 7.26-7.33 (t, 1H), 7.37-7.60 (m, 5H), 7.65 (d, 1H), 7.84-8.05 (br m, 3H), 8.14 (br s, 1H).

### EXAMPLE 17

### N′-(butylidene)-3-phenyl-1H-indole-2-carbohydrazide

The desired product was prepared by substituting butyraldehyde for 4-bromobenzaldehyde in Example 2. MS (ESI(+)) m/e 306 (M+H)⁺; ¹H NMR (300 MHz, DMSO-d₆) δ 0.85-0.95 (br m, 3H), 1.41-1.55 (br m, 2H), 2.14-2.25 (br m, 2H), 7.07-7.15 (m, 1H), 7.23-7.53 (m, 8H), 7.60 (d. 1H).

### EXAMPLE 18

### N′-(pentylidene)-3-phenyl-1H-indole-2-carbohydrazide

The desired product was prepared by substituting pentanal for 4-bromobenzaldehyde in Example 2. MS (ESI(+)) m/e 320 (M+H)⁺; ¹H NMR (300 MHz, DMSO-d₆) δ 0.80-0.93 (br m, 3H), 1.21-1.48 (m, 4H), 2.15-2.27 (br m, 2H), 7.06-7.14 (m, 1H), 7.20-7.53 (m, 7H), 7.60 (d, 1H).

### EXAMPLE 19

### N′-((4-chlorophenyl)methylidene)-3-phenyl-1H-indole-2-carbohydrazide

The desired product was prepared by substituting 4-chlorobenzaldehyde for 4-bromobenzaldehyde in Example 2. MS (ESI(+)) m/e 374 (M+H)⁺; ¹H NMR (300 MHz, DMSO-d₆) δ 7.10-7.16 (m, 1H), 7.26-7.33 (t, 1H), 7.37-7.77 (m, 11H), 8.06 (br s, 1H).

### EXAMPLE 20

### N′-((4-bromophenyl)methylidene)-3-isopropyl-1H-indole-2-carbohydrazide

### EXAMPLE 20A

### Ethyl 4-methyl-2-oxopentanoate

The desired product was prepared by substituting isobutylmagnesium bromide for benzylmagnesium chloride in Example 1A.

### EXAMPLE 20B

### Ethyl 3-isopropyl-1H-indole-2-carboxylate

The desired product was prepared by substituting Example 20A for Example 1A in Example 1B, then purifying the resulting product by flash column chromatography on silica gel with 0-10% ethyl acetate/n-hexane.

### EXAMPLE 20C

### 3-isopropyl-1H-indole-2-carbohydrazide

The desired product was prepared by substituting Example 20B for Example 1B in Example 1C, then purifying the resulting product by flash column chromatography on silica gel with 0-20% acetone/n-hexane.

### EXAMPLE 20D

### N′-((4-bromophenyl)methylidene)-3-isopropyl-1H-indole-2-carbohydrazide

The desired product was prepared by substituting Example 20C for Example 1C in Example 2. MS (ESI(−)) m/e 384 (M−H)⁻; ¹H NMR (300 MHz, DMSO-d₆) δ 1.40 (d, 6H), 3.82 (br m, 1H), 7.00-7.06 (m, 1H), 7.17-7.25 (m. 1H), 7.40-7.45 (m, 1H), 7.67 (s, 4H), 7.78-7.83 (m, 1H), 8.29 (br s, 1H).

### EXAMPLE 21

### N′-((4-chlorophenyl)methylidene)-3-isopropyl-1H-indole-2-carbohydrazide

The desired product was prepared by substituting Example 20C and 4-chlorobenzaldehyde for Example 1C and 4-bromobenzaldehyde, respectively, in Example 2. MS (ESI(+)) m/e 340 (M+H)⁺; ¹H NMR (300 MHz, DMSO-d₆) δ 1.40 (d, 6H), 3.82 (br m, 1H), 6.99-7.06 (m, 1H), 7.17-7.24 (m, 1H), 7.40-7.45 (m, 1H), 7.49-7.55 (m, 2H), 7.70-7.83 (m, 3H), 8.32 (br s, 1H).

### EXAMPLE 22

### N′-((4-fluorophenyl)methylidene)-3-isopropyl-1H-indole-2-carbohydrazide

The desired product was prepared by substituting Example 20C and 4-fluorobenzaldehyde for Example 1C and 4-bromobenzaldehyde, respectively, in Example 2. MS (ESI(+)) m/e 324 (M+H)⁺; ¹H NMR (300 MHz, DMSO-d₆) δ 1.39 (dd, 6H), 3.79 (br m,, 1H), 6.96-7.06 (m, 1H), 7.14-7.25 (m, 1H), 7.25-7.35 (m, 2H), 7.38-7.46 (m, 1H), 7.74-7.83 (m, 3H), 8.33 (br s, 1H).

### EXAMPLE 23

### N′-((4-bromophenyl)methylidene)-5-fluoro-3-phenyl-1H-indole-2-carbohydrazide

### EXAMPLE 23A

### Ethyl 5-fluoro-3-phenyl-1H-indole-2-carboxylate

The desired product was prepared by substituting 4-fluorophenylhydrazine for phenylhydrazine in Example 1B.

### EXAMPLE 23B

### 5-fluoro-3-phenyl-1H-indole-2-carbohydrazide

The desired product was prepared by substituting Example 23A for Example 1B in Example 1C.

### EXAMPLE 23C

### N′-((4-bromophenyl)methylidene)-5-fluoro-3-phenyl-1H-indole-2-carbohydrazide

The desired product was prepared by substituting Example 23B for Example 1C in Example 2. MS (ESI(+)) m/e 437 (M+H)⁺; ¹H NMR (300 MHz, DMSO-d₆) δ 7.11-7.19 (m, 2H), 7.30-7.36 (m, 1H), 7.42-7.56 (m, 6H), 7.59-7.70 (br s, 2H), 7.72-7.86 (m, 2H), 8.07 (br s, 1H).

### EXAMPLE 24

### N′-((4-chlorophenyl)methylidene)-5-fluoro-3-phenyl-1H-indole-2-carbohydrazide

The desired product was prepared by substituting Example 23B and 4-chlorobenzaldehyde for Example 1C and 4-bromobenzaldehyde, respectively, in Example 2. MS (ESI(+)) m/e 392 (M+H)⁺; ¹H NMR (300 MHz, DMSO-d₆) δ 7.11-7.20 (m, 2H), 7.29-7.56 (m, 8H), 7.65-7.77 (br m, 2H), 8.08 (br s, 1H).

### EXAMPLE 25

### 5-fluoro-N′-((4-fluorophenyl)methylidene)-3-phenyl-1H-indole-2-carbohydrazide

The desired product was prepared by substituting Example 23B and 4-fluorobenzaldehyde for Example 1C and 4-bromobenzaldehyde, respectively, in Example 2. MS (ESI(+)) m/e 376 (M+H)⁺; ¹H NMR (300 MHz, DMSO-d₆) δ 7.11-7.20 (m, 2H), 7.22-7.56 (m, 8H), 7.71-7.83 (br m, 2H), 8.08 (br s, 1H).

### EXAMPLE 26

### N′-((4-bromophenyl)methylidene)-5-methoxy-3-phenyl-1H-indole-2-carbohydrazide

### EXAMPLE 26A

### Ethyl 5-methoxy-3-phenyl-1H-indole-2-carboxylate

The desired product was prepared by substituting 4-methoxyphenylhydrazine for phenylhydrazine in Example 1B, collecting the resulting precipitate by filtration, and purifying the resulting product by recrystallization from ethanol.

### EXAMPLE 26B

### 5-methoxy-3-phenyl-1H-indole-2-carbohydrazide

The desired product was prepared by substituting Example 26A for Example 1B in Example 1C.

### EXAMPLE 26C

### N′-((4-bromophenyl)methylidene)-5-methoxy-3-phenyl-1H-indole-2-carbohydrazide

The desired product was prepared by substituting Example 26B for Example 1C in Example 2. MS (ESI(+)) m/e 450 (M+H)⁺; ¹H NMR (300 MHz, DMSO-d₆) δ 3.75 (s, 3H), 6.95 (dd, 1H), 7.26-7.70 (m, 11H), 8.03 (br s, 1H).

### EXAMPLE 27

### N′-((4-chlorophenyl)methylidene)-5-methoxy-3-phenyl-1H-indole-2-carbohydrazide

The desired product was prepared by substituting Example 26B and 4-chlorobenzaldehyde for Example 1C and 4-bromobenzaldehyde, respectively, in Example 2. MS (ESI(+)) m/e 404 (M+H)⁺; ¹H NMR (300 MHz, DMSO-d₆) δ 3.75 (s, 3H), 6.94 (dd, 1H), 7.03 (br m, 1H), 7.26-7.56 (m, 9H), 7.78 (br m, 1H), 8.05 (br s, 1H).

### EXAMPLE 28

### N′-((4-fluorophenyl)methylidene)-5-methoxy-3-phenyl-1H-indole-2-carbohydrazide

The desired product was prepared by substituting Example 26B and 4-fluorobenzaldehyde for Example 1C and 4-bromobenzaldehyde, respectively, in Example 2. MS (ESI(+)) m/e 388 (M+H)⁺; ¹H NMR (300 MHz, DMSO-d₆) δ 3.75 (s, 3H), 6.86-7.07 (m, 2H), 7.22-7.56 (m, 9H), 7.73 (br m, 1H), 8.05 (br s, 1H).

### EXAMPLE 29

### 5-bromo-N′-((4-bromophenyl)methylidene)-3-phenyl-1H-indole-2-carbohydrazide

### EXAMPLE 29A

### Ethyl 5-bromo-3-phenyl-1H-indole-2-carboxylate

The desired product was prepared by substituting 4-bromophenylhydrazine hydrochloride for phenylhydrazine in Example 1B, collecting the resulting precipitate by filtration, washing the solid with ethanol, and drying under vacuum.

### EXAMPLE 29B

### 5-bromo-3-phenyl-1H-indole-2-carbohydrazide

The desired product was prepared by substituting Example 29A for Example 1B in Example 1C.

### EXAMPLE 29C

### 5-bromo-N′-((4-bromophenyl)methylidene)-3-phenyl-1H-indole-2-carbohydrazide

The desired product was prepared by substituting Example 29B for Example 1C in Example 2. MS (ESI(+)) m/e 498 (M+H)⁺; ¹H NMR (300 MHz, DMSO-d₆) δ 7.15-7.56 (m, 9H), 7.59-7.77 (br m, 3H), 8.07 (br s, 1H).

### EXAMPLE 30

### 5-bromo-N′-((4-chlorophenyl)methylidene)-3-phenyl-1H-indole-2-carbohydrazide

The desired product was prepared by substituting Example 29B and 4-chlorobenzaldehyde for Example 1C and 4-bromobenzaldehyde, respectively, in Example 2. MS (ESI(+)) m/e 454 (M+H)⁺; ¹H NMR (300 MHz, DMSO-d₆) δ 7.25-7.57 (m, 9H), 7.66-7.77 (br s, 3H), 8.07 (br s, 1H).

### EXAMPLE 31

### 5-bromo-N′-((4-fluorophenyl)methylidene)-3-phenyl-1H-indole-2-carbohydrazide

The desired product was prepared by substituting Example 29B and 4-fluorobenzaldehyde for Example 1C and 4-bromobenzaldehyde, respectively, in Example 2. MS (ESI(−)) m/e 436(M+H)⁻; ¹H NMR (300 MHz, DMSO-d₆) δ 7.07-7.57 (m, 9H), 7.74 (br s, 3H), 8.08 (br s, 1H).

### EXAMPLE 32

### N′-((4-cyanophenyl)methylidene)-3-isopropyl-1H-indole-2-carbohydrazide

The desired product was prepared by substituting Example 20C and 4-cyanobenzaldehyde for Example 1C and 4-bromobenzaldehyde, respectively, in Example 2. MS (ESI(−)) m/e 329(M−H)⁻; ¹H NMR (300 MHz, DMSO-d₆) δ 1.40 (d, 6H), 3.83 (br m, 1H), 7.01-7.07 (dt, 1H), 7.18-7.26 (dt, 1H), 7.44 (d, 1H), 7.81 (d, 1H), 7.91 (s, 4H), 8.37 (br s, 1H).

### EXAMPLE 33

### N′-((4-cyanophenyl)methylidene)-5-fluoro-3-phenyl-1H-indole-2-carbohydrazide

The desired product was prepared by substituting Example 23B and 4-cyanobenzaldehyde for Example 1C and 4-bromobenzaldehyde, respectively, in Example 2. MS (ESI(+)) m/e 383 (M+H)⁺; ¹H NMR (300 MHz, DMSO-d₆) δ 7.13-7.22 (m, 1H), 7.24-7.60 (m, 7H), 7.68-8.22 (m, 5H).

### EXAMPLE 34

### N′-((4-cyanophenyl)methylidene)-5-methoxy-3-phenyl-1H-indole-2-carbohydrazide

The desired product was prepared by substituting Example 26B and 4-cyanobenzaldehyde for Example 1C and 4-bromobenzaldehyde, respectively, in Example 2. MS (ESI(+)) m/e 395 (M+H)⁺; ¹H NMR (300 MHz, DMSO-d₆) δ 3.75 (s, 3H), 6.93-6.97 (m, 1H), 7.04 (m, 1H), 7.22-7.56 (m, 6H), 7.60-7.95 (m, 4H), 8.07 (br s, 1H).

### EXAMPLE 35

### 5-bromo-N′-((4-cyanophenyl)methylidene)-3-phenyl-1H-indole-2-carbohydrazide

The desired product was prepared by substituting Example 29B and 4-cyanobenzaldehyde for Example 1C and 4-bromobenzaldehyde, respectively, in Example 2. MS (ESI(−)) m/e 443 (M−H)⁻; ¹H NMR (300 MHz, DMSO-d₆) δ 7.28-7.56 (m, 8H), 7.73 (br s, 1H), 7.78-7.94 (br m, 3H), 8.15 (br s, 1H).

### EXAMPLE 36

### 3-isopropyl-N′-(phenylmethylidene)-1H-indole-2-carbohydrazide

The desired product was prepared by substituting Example 20C and benzaldehyde for Example 1C and 4-bromobenzaldehyde, respectively, in Example 2. MS (ESI(−)) m/e 304 (M−H)⁻; ¹H NMR (300 MHz, DMSO-d₆) δ 1.40 (d, 6H), 3.83 (br m, 1H), 7.01-7.07 (dt, 1H), 7.18-7.25 (dt, 1H), 7.40-7.50 (m, 4H), 7.68-7.77 (br m, 2H), 7.81 (d, 1H), 8.33 (br s, 1H).

### EXAMPLE 37

### 5-fluoro-3-phenyl-N′-(phenylmethylidene)-1H-indole-2-carbohydrazide

The desired product was prepared by substituting Example 23B and benzaldehyde for Example 1C and 4-bromobenzaldehyde, respectively, in Example 2. MS (ESI(+)) m/e 358 (M+H)⁺; ¹H NMR (300 MHz, DMSO-d₆) δ 7.11-7.20 (m, 1H), 7.23-7.60 (m, 11H), 7.68 (br s, 1H), 8.08 (br s, 1H).

### EXAMPLE 38

### 5-methoxy-3-phenyl-N′-(phenylmethylidene)-1H-indole-2-carbohydrazide

The desired product was prepared by substituting Example 26B and benzaldehyde for Example 1C and 4-bromobenzaldehyde, respectively, in Example 2. MS (ESI(+)) m/e 370 (M+H)⁺; ¹H NMR (300 MHz, DMSO-d₆) δ 3.75 (s, 3H), 6.93-6.97 (m, 1H), 7.04 (s, 1H), 7.22-7.55 (m, 9H), 7.58-7.73 (m, 2H), 8.05 (br s, 1H).

### EXAMPLE 39

### 5-bromo-3-phenyl-N′-(phenylmethylidene)-1H-indole-2-carbohydrazide

The desired product was prepared by substituting Example 29B and benzaldehyde for Example 1C and 4-bromobenzaldehyde, respectively, in Example 2. MS (ESI(+)) m/e 420 (M+H)⁺; ¹H NMR (300 MHz, DMSO-d₆) δ 7.26-7.62 (m, 10H), 7.82-7.82 (m, 3H), 8.08 (br s, 1H).

### EXAMPLE 40

### 3-isopropyl-N′-((4-nitrophenyl)methylidene)-1H-indole-2-carbohydrazide

The desired product was prepared by substituting Example 20C and 4-nitrobenzaldehyde for Example 1C and 4-bromobenzaldehyde, respectively, in Example 2. MS (ESI(−)) m/e 349 (M−H)⁻; ¹H NMR (300 MHz, DMSO-d₆) δ 1.41 (d, 6H), 3.82 (br m, 1H), 7.01-7.07 (dt, 1H), 7.20-7.26 (dt, 1H), 7.44 (d, 1H), 7.82 (d, 1H), 7.98 (d, 2H), 8.32 (d, 2H), 8.43 (s, 1H).

### EXAMPLE 41

### 5-fluoro-N′-((4-nitrophenyl)methylidene)-3-phenyl-1H-indole-2-carbohydrazide

The desired product was prepared by substituting Example 23B and 4-nitrobenzaldehyde for Example 1C and 4-bromobenzaldehyde, respectively, in Example 2. MS (ESI(−)) m/e 401 (M−H)^{-;} ¹H NMR (300 MHz, DMSO-d₆) δ 7.13-7.22 (m, 1H), 7.25-7.57 (m, 6H), 7.85-8.40 (m, 5H).

### EXAMPLE 42

### 5-methoxy-N′-((4-nitrophenyl)methylidene)-3-phenyl-1H-indole-2-carbohydrazide

The desired product was prepared by substituting Example 26B and 4-nitrobenzaldehyde for Example 1C and 4-bromobenzaldehyde, respectively, in Example 2. MS (ESI(+)) m/e 415 (M+H)⁺; ¹H NMR (300 MHz, DMSO-d₆) δ 3.75 (s, 3H), 6.94-6.99 (m, 1H), 7.05 (s, 1H), 7.22-7.57 (m, 6H), 7.73-8.32 (m, 5H).

### EXAMPLE 43

### 5-bromo-N′-((4-nitrophenyl)methylidene)-3-phenyl-1H-indole-2-carbohydrazide

The desired product was prepared by substituting Example 29B and 4-nitrobenzaldehyde for Example 1C and 4-bromobenzaldehyde, respectively, in Example 2. MS (ESI(+)) m/e 463 (M+H)⁺; ¹H NMR (300 MHz, DMSO-d₆) δ 7.35-7.54 (m, 7H), 7.75 (br s, 1H), 7.88-8.03 (br m, 1H), 8.03-8.41 (br m, 4H).

### EXAMPLE 44

### N′-(1-naphthylmethylidene)-3-phenyl-1H-indole-2-carbohydrazide

The desired product was prepared by substituting 1-naphthaldehdye for 4-bromobenzaldehyde in Example 2. MS (ESI(+)) m/e 390 (M+H)⁺; ¹H NMR (300 MHz, DMSO-d₆) δ 7.12-7.17 (m, 1H), 7.26-7.41 (m, 2H), 7.41-7.76 (m, 10H), 7.82-8.08 (br m, 3H), 8.73 (br s, 1H).

### EXAMPLE 45

### 3-phenyl-N′-((4-(trifluoromethyl)phenyl)methylidene)-1H-indole-2-carbohydrazide

The desired product was prepared by substituting 4-(trifluoromethyl)benzaldehyde for 4-bromobenzaldehyde in Example 2. MS (ESI(+)) m/e 408 (M+H)⁺.

### EXAMPLE 46

### 3-phenyl-N′-(4-quinolinylmethylidene)-1H-indole-2-carbohydrazide

The desired product was prepared by substituting 4-quinolinecarbaldehyde for 4-bromobenzaldehyde in Example 2. MS (ESI(+)) m/e 391 (M+H)⁺; ¹H NMR (300 MHz, DMSO-d₆) δ 7.12-7.20 (m, 2H), 7.28-7.86 (m, 10H), 8.08 (br s, 1H), 8.70 (br s, 1H), 8.95 (br s, 1H).

### EXAMPLE 47

### Methyl 4-((((3-phenyl-1H-indol-2-yl)carbonyl)hydrazono)methyl)benzoate

The desired product was prepared by substituting ethyl 4-formylbenzoate for 4-bromobenzaldehyde in Example 2. MS (ESI(+)) m/e 398 (M+H)⁺; ¹H NMR (300 MHz, DMSO-d₆) δ 3.87 (s, 3H), 7.11-7.16 (m, 1H), 7.27-7.35 (m, 2H), 7.38-8.17 (br m, 11H).

### EXAMPLE 48

### N′-((4-bromophenyl)methylidene)-3-(4-fluorophenyl)-1H-indole-2-carbohydrazide

### EXAMPLE 48A

### Ethyl 3-(4-fluorophenyl)-2-oxopropanoate

The desired product was prepared by substituting 4-fluorobenzylmagnesium bromide for benzylmagnesium chloride in Example 1A.

### EXAMPLE 48B

### Ethyl 3-(4-fluorophenyl)-1H-indole-2-carboxylate

The desired product was prepared by substituting Example 48A for Example 1A in Example 1B, collecting the resulting precipitate by filtration, washing the solid with ethanol, and drying under vacuum.

### EXAMPLE 48C

### 3-(4-fluorophenyl)-1H-indole-2-carbohydrazide

The desired product was prepared by substituting Example 48B for Example 1B in Example 1C.

### EXAMPLE 48D

### N′-((4-bromophenyl)methylidene)-3-(4-fluorophenyl)-1H-indole-2-carbohydrazide

The desired product was prepared by substituting Example 48C for Example 1C in Example 2. MS (ESI(+)) m/e 438 (M+H)⁺; ¹H NMR (300 MHz, DMSO-d₆) δ 7.11-7.17 (m, 1H), 7.19-7.35 (m, 3H), 7.48-7.73 (m, 7H), 8.10 (br s, 1H).

### EXAMPLE 49

### N′-((4-chlorophenyl)methylidene)-3-(4-fluorophenyl)-1H-indole-2-carbohydrazide

The desired product was prepared by substituting Example 48C and 4-chlorobenzaldehyde for Example 1C and 4-bromobenzaldehyde, respectively, in Example 2. MS (ESI(+)) m/e 392 (M+H)⁺; ¹H NMR (300 MHz, DMSO-d₆) δ 7.12-7.17 (m, 1H), 7.19-7.35 (m, 3H), 7.38-7.65 (m, 5H), 7.66-7.81 (m, 4H), 8.11 (br s, 1H).

### EXAMPLE 50

### 3-(4-fluorophenyl)-N′-((4-nitrophenyl)methylidene)-1H-indole-2-carbohydrazide

The desired product was prepared by substituting Example 48C and 4-nitrobenzaldehyde for Example 1C and 4-bromobenzaldehyde, respectively, in Example 2. MS (ESI(+)) m/e 403 (M+H)⁺; ¹H NMR (300 MHz, DMSO-d₆) δ 7.12-7.17 (m, 1H), 7.19-7.35 (m, 3H), 7.48-7.57 (m, 3H), 7.76-8.35 (m, 4H), 11.64-11.98 (br m, 2H).

### EXAMPLE 51

### N′-((4-cyanophenyl)methylidene)-3-(4-fluorophenyl)-1H-indole-2-carbohydrazide

The desired product was prepared by substituting Example 48C and 4-cyanobenzaldehyde for Example 1C and 4-bromobenzaldehyde, respectively, in Example 2. MS (ESI(+)) m/e 383 (M+H)⁺; ¹H NMR (300 MHz, DMSO-d₆) δ 7.12-7.17 (m, 1H), 7.19-7.33 (m, 2H), 7.48-7.57 (m, 3H), 7.62 (d, 1H), 7.74-7.96 (br m, 4H), 8.14 (br s, 1H).

### EXAMPLE 52

### 3-(4-chlorophenyl)-N′-((4-cyanophenyl)methylidene)-1H-indole-2-carbohydrazide

### EXAMPLE 52A

### Ethyl 3-(4-chlorophenyl)-2-oxopropanoate

The desired product was prepared by substituting 4-chlorobenzylmagnesium bromide for benzylmagnesium chloride in Example 1A.

### EXAMPLE 52B

### Ethyl 3-(4-chlorophenyl)-1H-indole-2-carboxylate

A mixture of Example 52A (4.75 g, 21.0 mmol) and phenyl hydrazine (2.07 mL, 21.0 mmol) was treated with concentrated sulfuric acid (5 drops), heated to 120° C. for 15 minutes, cooled to room temperature, treated with ethanol (25 mL), treated with bubbling HCl gas for 2 minutes, and heated to reflux for 1 hour. The mixture was poured into water (30 mL) and extracted with diethyl ether. The combined extracts were washed with water and brine, dried (MgSO₄), filtered, and concentrated. The concentrate was recrystallized from ethanol to provide 750 mg (12%) of the desired product.

### EXAMPLE 52C

### 3-(4-chlorophenyl)-1H-indole-2-carbohydrazide

The desired product was prepared by substituting Example 52B for Example 1B in Example 1C.

### EXAMPLE 52D

### 3-(4-chlorophenyl)-N′-((4-cyanophenyl)methylidene)-1H-indole-2-carbohydrazide

The desired product was prepared by substituting Example 52C and 4-cyanobenzaldehyde for Example 1C and 4-bromobenzaldehyde, respectively, in Example 2. MS (ESI(+)) m/e 399 (M+H)⁺; ¹H NMR (300 MHz, DMSO-d₆) δ 7.12-7.18 (m, 1H), 7.27-7.33 (m, 1H), 7.39-7.59 (m, 5H), 7.65 (d, 1H), 7.79-7.97 (m, 3H), 8.18 (br s, 1H).

### EXAMPLE 53

### N′-((4-bromophenyl)methylidene)-3-(4-chlorophenyl)-1H-indole-2-carbohydrazide

The desired product was prepared by substituting Example 52C for Example 1C in Example 2. MS (ESI(−)) m/e 452 (M−H)⁻; ¹H NMR (300 MHz, DMSO-d₆) δ 7.12-7.17 (t, 1H), 7.27-7.33 (t, 1H), 7.43-7.74 (m, 10H), 8.13 (br s, 1H).

### EXAMPLE 54

### 3-(4-chlorophenyl)-N′-((4-chlorophenyl)methylidene)-1H-indole-2-carbohydrazide

The desired product was prepared by substituting Example 52C and 4-chlorobenzaldehyde for Example 1C and 4-bromobenzaldehyde, respectively, in Example 2. MS (ESI(−)) m/e 406 (M−H)⁻; ¹H NMR (300 MHz, DMSO-d₆) δ 7.12-7.18 (m, 1H), 7.27-7.33 (t, 1H), 7.41-7.58 (m, 8H), 7.64 (d, 1H), 7.68-7.79 (br m, 1H), 8.14 (br s, 1H).

### EXAMPLE 55

### 3-(4-chlorophenyl)-N′-((4-nitrophenyl)methylidene)-1H-indole-2-carbohydrazide

The desired product was prepared by substituting Example 52C and 4-nitrobenzaldehyde for Example 1C and 4-bromobenzaldehyde, respectively, in Example 2. MS (ESI(−)) m/e 417 (M−H)⁻; ¹H NMR (300 MHz, DMSO-d₆) δ 7.12-7.18 (m, 1H), 7.27-7.35 (t, 1H), 7.40-7.56 (m, 8H), 7.65 (d, 1H), 7.85-8.05 (br m, 2H), 8.15-8.34 (br m, 3H).

### EXAMPLE 56

### N′-((4-bromo-3,5-dimethoxyphenyl)methylidene)-3-phenyl-1H-indole-2-carbohydrazide

The desired product was prepared by substituting 4-bromo-3,5-dimethoxybenzaldehyde for 4-bromobenzaldehyde in Example 2. MS (ESI(−)) m/e 478 (M−H)⁻; ¹H NMR (300 MHz, DMSO-d₆) δ 3.87 (br s, 6H), 7.02 (br s, 1H), 7.09-7.17 (t, 1H), 7.25-7.56 (m, 8H), 7.65 (d, 1H), 8.05 (br s, 1H).

### EXAMPLE 57

### N′-((3,4-dichlorophenyl)methylidene)-3-phenyl-1H-indole-2-carbohydrazide

The desired product was prepared by substituting 3,4-dichlorobenzaldehyde for 4-bromobenzaldehyde in Example 2. MS (ESI(+)) m/e 408 (M+H)⁺; ¹H NMR (300 MHz, DMSO-d₆) δ 7.09-7.17 (m, 1H), 7.26-7.33 (m, 1H), 7.38-8.10 (m, 10H).

### EXAMPLE 58

### N′-((4-bromo-2-fluorophenyl)methylidene)-3-phenyl-1H-indole-2-carbohydrazide

The desired product was prepared by substituting 4-bromo-2-fluorobenzaldehyde for 4-bromobenzaldehyde in Example 2. MS (ESI(+)) m/e 438 (M+H)⁺; ¹H NMR (300 MHz, DMSO-d₆) δ 7.09-7.17 (m, 1H), 7.26-7.34 (t, 1H), 7.38-7.68 (m, 10H), 8.25 (br s, 1H), 11.92 (br s, 1H).

### EXAMPLE 59

### N′-((2,4-dichlorophenyl)methylidene)-3-phenyl-1H-indole-2-carbohydrazide

The desired product was prepared by substituting 2,4-dichlorobenzaldehyde for 4-bromobenzaldehyde in Example 2. MS (ESI(+)) m/e 408 (M+H)⁺; ¹H NMR (300 MHz, DMSO-d₆) δ 7.09-7.17 (m, 1H), 7.26-7.34 (t, 1H), 7.38-7.56 (m, 9H), 7.63-7.68 (d, 1H), 8.45 (br s, 1H), 11.92 (br s, 1H).

### EXAMPLE 60

### N′-((4-chloro-3-nitrophenyl)methylidene)-3-phenyl-1H-indole-2-carbohydrazide

The desired product was prepared by substituting 4-chloro-3-nitrobenzaldehyde for 4-bromobenzaldehyde in Example 2. MS (ESI(−)) m/e 417 (M−H)⁻; ¹H NMR (300 MHz, DMSO-d₆) δ 7.10-7.17 (m, 1H), 7.26-7.35 (t, 1H), 7.37-8.40 (m, 10H), 8.13 (br s, 1H), 11.92 (br s, 1H).

### EXAMPLE 61

### N′-((4-fluorophenyl)methylidene)-3-methyl-1H-indole-2-carbohydrazide

### EXAMPLE 61A

### Ethyl 2-oxobutanoate

The desired product was prepared by substituting ethylmagnesium bromide for benzylmagnesium chloride in Example 1A.

### EXAMPLE 61B

### Ethyl 3-methyl-1H-indole-2-carboxylate

The desired product was prepared by substituting Example 61A for Example 1A in Example 1B, then purifying the resulting product by flash column chromatography on silica gel with 0-10% ethyl acetate/hexanes. ¹H NMR (300 MHz, DMSO-d₆) δ 1.36 (t, 3H), 2.54 (s, 3H), 4.34 (q, 2H), 7.05 (t, 1H), 7.25 (t, 1H), 7.40 (d, 1H), 7.64 (d, 1H), 11.44 (s, 1H).

### EXAMPLE 61C

### 3-methyl-1H-indole-2-carbohydrazide

The desired product was prepared by substituting Example 61B for Example 1B in Example 1C, then purifying the resulting product by flash column chromatography on silica gel with 0-20% ethyl acetate/hexanes. MS (ESI(+)) m/e 190 (M+H)⁺; ¹H NMR (300 MHz, DMSO-d₆) δ 2.47 (s, 3H), 4.49 (s, 2H), 7.03 (t, 1H), 7.18 (t, 1H), 7.36 (d, 1H), 7.58 (d, 1H), 9.12 (s, 1H), 11.07 (s, 1H).

### EXAMPLE 61D

### N′-((4-fluorophenyl)methylidene)-3-methyl-1H-indole-2-carbohydrazide

The desired product was prepared by substituting Example 61C and 4-fluorobenzaldehyde for Example 1C and 4-bromobenzaldehyde, respectively, in Example 2. MS (ESI(+)) m/e 296 (M+H)⁺; ¹H NMR (300 MHz, DMSO-d₆) δ 2.54 (s, 3H), 7.70 (t, 1H), 7.22-7.35 (m, 3H), 7.43 (d, 1H), 7.64 (d, 1H), 7.80 (m, 2H), 8.36 (br s, 1H).

### EXAMPLE 62

### N′-((4-cyanophenyl)methylidene)-3-(3,4-dimethylphenyl)-1H-indole-2-carbohydrazide

### EXAMPLE 62A

### Ethyl 3-(3,4-dimethylphenyl)-2-oxopropanoate

The desired product was prepared by substituting 3,4-dimethylbenzylmagnesium bromide for benzylmagnesium chloride in Example 1A.

### EXAMPLE 62B

### Ethyl 3-(3,4-dimethylphenyl)-1H-indole-2-carboxylate

The desired product was prepared by substituting Example 62A for Example 1A in Example 1B, then purifying the resulting product by flash column chromatography on silica gel with 0-10% ethyl acetate/hexanes. MS (ESI(−)) m/e 292 (M−H)⁻; ¹H NMR (300 MHz, DMSO-d₆) δ 1.20 (t, 3H), 2.28 (s, 6H), 4.22 (q, 2H), 7.07 (t, 1H), 7.20 (s, 2H), 7.27 (s, 1H), 7.29 (t, 1H), 7.48 (d, 2H), 11.81 (s, 1H).

### EXAMPLE 62C

### 3-(3,4-dimethylphenyl)-1H-indole-2-carbohydrazide

The desired product was prepared by substituting Example 62B for Example 1B in Example 1C, then purifying the resulting product by recrystallization from ethanol.

### EXAMPLE 62D

### N′-((4-cyanophenyl)methylidene)-3-(3,4-dimethylphenyl)-1H-indole-2-carbohydrazide

The desired product was prepared by substituting Example 62C and 4-cyanobenzaldehyde for Example 1C and 4-bromobenzaldehyde in Example 2. MS (ESI(+)) m/e 393 (M+H)⁺; ¹H NMR (300 MHz, DMSO-d₆) δ 2.22 (br s, 6H), 7.08-7.18 (m, 3H), 7.25-7.31 (m, 3H), 7.49 (d, 1H), 7.61 (d, 1H), 7.85 (br s, 3H), 8.08 (br s, 1H).

### EXAMPLE 63

### N′-((4-chlorophenyl)methylidene)-3-(3,4-dimethylphenyl)-1H-indole-2-carbohydrazide

The desired product was prepared by substituting Example 62C and 4-chlorobenzaldehyde for Example 1C and 4-bromobenzaldehyde in Example 2. MS (ESI(+)) m/e 402 (M+H)⁺; ¹H NMR (300 MHz, DMSO-d₆) δ 2.25 (br s, 6H), 7.10 (t, 1H), 7.20 (br s, 2H), 7.24-7.32 (m, 3H), 7.48 (m, 3H), 7.60 (d, 1H), 7.68 (br s, 1H), 8.04 (br s, 1H).

### EXAMPLE 64

### 3-(3,4-dimethylphenyl)-N′-((4-nitrophenyl)methylidene)-1H-indole-2-carbohydrazide

The desired product was prepared by substituting Example 62C and 4-nitrobenzaldehyde for Example 1C and 4-bromobenzaldehyde in Example 2. MS (ESI(+)) m/e 413 (M+H)⁺; ¹H NMR (300 MHz, DMSO-d₆) δ 2.22 (br s, 6H), 7.12 (t, 2H), 7.19 (br s, 2H), 7.26-7.32 (m, 2H), 7.50 (d, 1H), 7.63 (d, 1H), 7.90 (br s, 1H), 8.02 (br s, 1H), 8.25 (m, 2H).

### EXAMPLE 65

### 3-(3,4-dimethylphenyl)-N′-((4-fluorophenyl)methylidene)-1H-indole-2-carbohydrazide

The desired product was prepared by substituting Example 62C and 4-fluorobenzaldehyde for Example 1C and 4-bromobenzaldehyde, respectively, in Example 2. MS (ESI(+)) m/e 386 (M+H)⁺; ¹H NMR (300 MHz, DMSO-d₆) δ 2.28 (br s, 6H), 7.10 (t, 1H), 7.20-7.32 (m, 6H), 7.49 (d, 1H), 7.61 (d, 1H), 7.73 (br s, 1H), 8.05 (br s, 1H).

### EXAMPLE 66

### 3-phenyl-N′-(4-pyridinylmethylidene)-1H-indole-2-carbohydrazide

The desired product was prepared by substituting isonicotinaldehyde for 4-bromobenzaldehyde in Example 2. MS (ESI(−)) m/e 339 (M−H)⁻; ¹H NMR (300 MHz, DMSO-d₆) δ 7.10-7.17 (m, 2H), 7.26-7.33 (t, 2H), 7.35-7.68 (m, 8H), 8.03 (br s, 1H), 8.58 (br s, 2H).

### EXAMPLE 67

### 3-phenyl-N′-(3-pyridinylmethylidene)-1H-indole-2-carbohydrazide

The desired product was prepared by substituting nicotinaldehyde for 4-bromobenzaldehyde in Example 2. MS (ESI(+)) m/e 341 (M+H)⁺; ¹H NMR (300 MHz, DMSO-d₆) δ 7.09-7.17 (m, 2H), 7.24-7.57 (m, 10H), 7.61 (d, 1H), 8.13 (br s, 1H), 8.58 (br s, 1H).

### EXAMPLE 68

### 3-phenyl-N′-(2-pyridinylmethylidene)-1H-indole-2-carbohydrazide

The desired product was prepared by substituting 2-pyridinecarbaldehyde for 4-bromobenzaldehyde in Example 2. MS (ESI(+)) m/e 341 (M+H)⁺; ¹H NMR (300 MHz, DMSO-d₆) δ 7.11-7.18 (m, 2H), 7.26-7.61 (m, 10H), 7.65 (d, 1H), 8.03 (br s, 1H), 8.57 (br s, 1H).

### EXAMPLE 69

### N′-((6-methyl-2-pyridinyl)methylidene)-3-phenyl-1H-indole-2-carbohydrazide

The desired product was prepared by substituting 6-methyl-2-pyridinecarbaldehyde for 4-bromobenzaldehyde in Example 2. MS (ESI(+)) m/e 355 (M+H)⁺; ¹H NMR (300 MHz, DMSO-d₆) δ 2.46 (s, 3H), 7.09-7.17 (m, 2H), 7.22-7.40 (m, 4H), 7.42-7.57 (m, 4H), 7.63-7.79 (m, 2H), 8.01 (br s, 1H).

### EXAMPLE 70

### 3-methyl-N′-(phenylmethylidene)-1H-indole-2-carbohydrazide

The desired product was prepared by substituting Example 61C and benzaldehyde for Example 1C and 4-bromobenzaldehyde, respectively, in Example 2.

### EXAMPLE 71

### N′-((4-bromophenyl)methylidene)-3-methyl-1H-indole-2-carbohydrazide

The desired product was prepared by substituting Example 61C for Example 1C in Example 2.

### EXAMPLE 72

### N′-((4-cyanophenyl)methylidene)-3-methyl-1H-indole-2-carbohydrazide

The desired product was prepared by substituting Example 61C and 4-cyanobenzaldehyde for Example 1C and 4-bromobenzaldehyde, respectively, in Example 2.

### EXAMPLE 73

### N′-(3-furylmethylidene)-3-phenyl-1H-indole-2-carbohydrazide

The desired product was prepared by substituting 3-furaldehyde for 4-bromobenzaldehyde in Example 2. MS (ESI(+)) m/e 330 (M+H)⁺; ¹H NMR (300 MHz, DMSO-d₆) δ 6.78 (br s, 1H), 7.12 (t, 1H), 7.24-7.36 (m, 3H), 7.41-7.55 (m, 4H), 7.63 (d, 1H), 7.75 (br s, 1H), 8.02 (s, 1H), 8.12 (s, 1H).

### EXAMPLE 74

### N′-((5-methyl-2-furyl)methylidene)-3-phenyl-1H-indole-2-carbohydrazide

The desired product was prepared by substituting 5-methyl-2-furaldehyde for 4-bromobenzaldehyde in Example 2. MS (ESI(+)) m/e 344 (M+H)⁺; ¹H NMR (300 MHz, DMSO-d₆) δ 2.33 (s, 3H), 6.24 (s, 1H), 6.77 (s, 1H), 7.13 (t, 1H), 7.24-7.55 (m, 7H), 7.64 (d, 1H), 7.84 (br s, 1H).

### EXAMPLE 75

### N′-(1-benzofuran-2-ylmethylidene)-3-phenyl-1H-indole-2-carbohydrazide

The desired product was prepared by substituting 1-benzofuran-2-carbaldehyde for 4-bromobenzaldehyde in Example 2. MS (ESI(+)) m/e 380 (M+H)⁺; ¹H NMR (300 MHz, DMSO-d₆) δ 7.11-7.17 (t, 2H), 7.24-7.55 (m, 10H), 7.61-7.72 (d, 4H), 8.08 (br s, 1H).

### EXAMPLE 76

### N′-((5-nitro-2-furyl)methylidene)-3-phenyl-1H-indole-2-carbohydrazide

The desired product was prepared by substituting 5-nitro-2-furaldehyde for 4-bromobenzaldehyde in Example 2. MS (ESI(−)) m/e 373 (M−H)⁻; ¹H NMR (300 MHz, DMSO-d₆) δ 7.13 (t, 1H), 7.28-7.35 (m, 2H), 7.37-7.55 (m, 4H), 7.65 (d, 1H), 7.76 (s, 1H), 7.97 (br s, 1H).

### EXAMPLE 77

### N′-(2-furylmethylidene)-3-phenyl-1H-indole-2-carbohydrazide

The desired product was prepared by substituting 2-furaldehyde for 4-bromobenzaldehyde in Example 2. MS (ESI(+)) m/e 330 (M+H)⁺; ¹H NMR (300 MHz, DMSO-d₆) δ 6.62 (br s, 1H), 6.90 (br s, 1H), 7.12 (t, 1H), 7.24-7.37 (m, 3H), 7.39-7.55 (m, 4H), 7.64 (d, 1H), 7.83 (s, 1H), 7.94 (s, 1H).

### EXAMPLE 78

### 3-isopropyl-N′-((5-nitro-2-furyl)methylidene)-1H-indole-2-carbohydrazide

The desired product was prepared by substituting Example 20C and 5-nitro-2-furaldehyde for Example 1C and 4-bromobenzaldehyde, respectively, in Example 2. MS (ESI(−) m/e 339 (M−H)⁻; ¹H NMR (300 MHz, DMSO-d₆) δ 1.41 (d, 6H), 3.80 (m, 1H), 7.02-7.07 (m, 1H), 7.20-7.29 (m, 2H), 7.43 (d, 1H), 7.79-7.85 (m, 2H), 8.27 (s, 1H).

### EXAMPLE 79

### 3-isopropyl-N′-((5-methyl-2-furyl)methylidene)-1H-indole-2-carbohydrazide

The desired product was prepared by substituting Example 20C and 5-methyl-2-furaldehyde for Example 1C and 4-bromobenzaldehyde, respectively, in Example 2. MS (ESI(+)) m/e 310 (M+H)⁺; ¹H NMR (300 MHz, DMSO-d₆) δ 1.40 (d, 6H), 2.36 (s, 3H), 3.78 (br m, 1H), 6.27 (m, 1H), 6.81 (d, 1H), 6.97-7.06 (m, 1H), 7.16-7.23 (m, 1H), 7.41 (d, 1H), 7.78 (d, 1H), 8.10 (br s, 1H).

### EXAMPLE 80

### 3-isopropyl-N′-(3-pyridinylmethylidene)-1H-indole-2-carbohydrazide

The desired product was prepared by substituting Example 20C and nicotinaldehyde for Example 1C and 4-bromobenzaldehyde, respectively, in Example 2. MS (ESI(+)) m/e 307 (M+H)⁺; ¹H NMR (300 MHz, DMSO-d₆) δ 1.41 (d, 6H), 3.83 (br m., 1H), 7.00-7.06 (m, 1H), 7.18-7.25 (m, 1H), 7.40-7.53 (m, 2H), 7.80 (d, 1H), 8.13 (m, 1H), 8.36 (br s, 1H), 8.62 (m, 1H), 8.87 (d, 1H).

### EXAMPLE 81

### N′-(2-furylmethylidene)-3-isopropyl-1H-indole-2-carbohydrazide

The desired product was prepared by substituting Example 20C and 2-furaldehyde for Example 1C and 4-bromobenzaldehyde, respectively, in Example 2. MS (ESI(+)) m/e 296 (M+H)⁺; ¹H NMR (300 MHz, DMSO-d₆) δ 1.40 (d, 6H), 3.79 (br s, 1H), 6.65 (m, 1H), 6.94 (d, 1H), 7.00-7.06 (m, 1H), 7.16-7.23 (m, 1H), 7.42 (d, 1H), 7.78 (d, 1H), 7.85 (s, 1H), 8.20 (br s, 1H).

### EXAMPLE 82

### 3-methyl-N′-((5-nitro-2-furyl)methylidene)-1H-indole-2-carbohydrazide

The desired product was prepared by substituting Example 61C and 5-nitro-2-furaldehyde for Example 1C and 4-bromobenzaldehyde, respectively, in Example 2. MS (ESI(−)) m/e 311 (M−H)⁻; ¹H NMR (300 MHz, DMSO-d₆) δ 2.54 (s, 3H), 7.05-7.12 (m, 1H), 7.23-7.29 (m, 1H), 7.43 (d, 1H), 7.66 (d, 1H), 7.81 (d, 1H), 8.31 (s, 1H).

### EXAMPLE 83

### 3-methyl-N′-((5-methyl-2-furyl)methylidene)-1H-indole-2-carbohydrazide

The desired product was prepared by substituting Example 61C and 5-methyl-2-furaldehyde for Example 1C and 4-bromobenzaldehyde, respectively, in Example 2. MS (ESI(+)) m/e 282 (M+H)⁺; ¹H NMR (300 MHz, DMSO-d₆) δ 2.36 (s, 3H), 2.53 (s, 3H), 6.28 (m, 1H), 6.83 (d, 1H), 7.04-7.10 (m, 1H), 7.20-7.27 (m, 1H), 7.42 (d, 1H), 7.63 (d, 1H), 8.14 (br s, 1H).

### EXAMPLE 84

### 3-methyl-N′-(3-pyridinylmethylidene)-1H-indole-2-carbohydrazide

The desired product was prepared by substituting Example 61C and nicotinaldehyde for Example 1C and 4-bromobenzaldehyde, respectively, in Example 2. MS (ESI(+)) m/e 279 (M+H)⁺; ¹H NMR (300 MHz, DMSO-d₆) δ 2.53 (s, 3H), 6.65 (m, 1H), 7.05-7.11 (m, 1H), 7.22-7.28 (m, 1H), 7.42-7.53 (m, 2H), 7.65 (d, 1H), 8.15 (m, 1H), 8.40 (br s, 1H), 8.62 (m, 1H), 8.88 (d, 1H).

### EXAMPLE 85

### N′-(2-furylmethylidene)-3-methyl-1H-indole-2-carbohydrazide

The desired product was prepared by substituting Example 61C and 2-furaldehyde for Example 1C and 4-bromobenzaldehyde, respectively, in Example 2. MS (ESI(+)) m/e 268 (M+H)⁺; ¹H NMR (300 MHz, DMSO-d₆) δ 2.53 (s, 3H), 6.65 (m, 1H), 6.95 (d, 1H), 7.04-7.10 (m, 1H), 7.21-7.27 (m, 1H), 7.43 (d, 1H), 7.63 (d, 1H), 7.86 (m, 1H), 8.24 (br s, 1H).

### EXAMPLE 86

### 3-phenyl-N′-(1,3-thiazol-2-ylmethylidene)-1H-indole-2-carbohydrazide

The desired product was prepared by substituting 1,3-thiazole-2-carbaldehyde for 4-bromobenzaldehyde in Example 2. MS (ESI(−)) m/e 345 (M−H)⁻; ¹H NMR (300 MHz, DMSO-d₆) δ 7.10-7.17 (m, 1H), 7.26-7.36 (m, 2H), 7.38-7.57 (m, 4H), 7.65 (d, 1H), 7.83 (br s, 1H), 7.93 (s, 1H), 8.27 (br s, 1H).

### EXAMPLE 87

### N′-((4,5-dimethyl-2-furyl)methylidene)-3-phenyl-1H-indole-2-carbohydrazide

The desired product was prepared by substituting 4,5-dimethyl-2-furaldehyde for 4-bromobenzaldehyde in Example 2. MS (ESI(+)) m/e 358 (M+H)⁺; ¹H NMR (300 MHz, DMSO-d₆) δ 1.93 (s, 3H), 2.25 (s, 3H), 6.68 (s, 1H), 7.08-7.15 (m, 1H), 7.24-7.38 (m, 2H), 7.40-7.54 (m, 4H), 7.64 (d, 1H), 7.78 (s, 1H).

### EXAMPLE 88

### N′-((5-(4-chlorophenyl)-2-furyl)methylidene)-3-phenyl-1H-indole-2-carbohydrazide

The desired product was prepared by substituting 5-(4-chlorophenyl)-2-furaldehyde for 4-bromobenzaldehyde in Example 2. MS (ESI(+)) m/e 440 (M+H)⁺; ¹H NMR (300 MHz, DMSO-d₆) δ 7.03 (br s, 1H), 7.10-7.20 (m, 2H), 7.26-7.49 (m, 3H), 7.40-7.57 (m, 6H), 7.65 (d, 1H), 7.76-7.86 (br m, 2H), 7.98 (br s, 1H).

### EXAMPLE 89

### N′-((5-ethyl-2-furyl)methylidene)-3-phenyl-1H-indole-2-carbohydrazide

The desired product was prepared by substituting 5-ethyl-2-furaldehyde for 4-bromobenzaldehyde in Example 2. MS (ESI(+)) m/e 358 (M+H)⁺; ¹H NMR (300 MHz, DMSO-d₆) δ 1.19 (t, 3H), 2.68 (br q, 2H), 6.25 (br s., 1H), 6.78 (br s, 1H), 7.09-7.16 (m, 1H), 7.24-7.38 (m, 2H), 7.40-7.56 (m, 5H), 7.63 (d, 1H), 7.85 (br s, 1H).

### EXAMPLE 90

### (5-((2-((3-phenyl-1H-indol-2-yl)carbonyl)hydrazono)methyl)-2-furyl)methyl Acetate

The desired product was prepared by substituting (5-formyl-2-furyl)methyl acetate for 4-bromobenzaldehyde in Example 2. MS (ESI(−)) m/e 400 (M−H)⁻; ¹H NMR (300 MHz, DMSO-d₆) δ 2.06 (s, 3H), 5.06 (s, 2H), 6.65 (br s, 1H), 6.87 (br s, 1H), 7.09-7.16 (m, 1H), 7.25-7.39 (m, 2H), 7.39-7.56 (m, 5H), 7.65 (d, 1H), 7.90 (br s, 1H).

### EXAMPLE 91

### N′-((5-(4-nitrophenyl)-2-furyl)methylidene)-3-phenyl-1H-indole-2-carbohydrazide

The desired product was prepared by substituting 5-(4-nitrophenyl)-2-furaldehyde for 4-bromobenzaldehyde in Example 2. MS (ESI(−)) m/e 449 (M−H)⁻; ¹H NMR (300 MHz, DMSO-d₆) δ 7.10-8.37 (m, 15H).

### EXAMPLE 92

### N′-((4-methyl-1H-imidazol-5-yl)methylidene)-3-phenyl-1H-indole-2-carbohydrazide

The desired product was prepared by substituting 4-methyl-1H-imidazole-5-carbaldehyde for 4-bromobenzaldehyde in Example 2. MS (ESI(+)) m/e 344 (M+H)⁺; ¹H NMR (300 MHz, DMSO-d₆) δ 2.26 (s, 3H), 7.05-7.13 (m, 1H), 7.16-7.33 (m, 4H), 7.35-7.46 (m, 2H), 7.46-7.57 (m, 4H).

### EXAMPLE 93

### N′(1H-imidazol-2-ylmethylidene)-3-phenyl-1H-indole-2-carbohydrazide

The desired product was prepared by substituting 1H-imidazole-2-carbaldehyde for 4-bromobenzaldehyde in Example 2. MS (ESI(+)) m/e 330 (M+H)⁺; ¹H NMR (300 MHz, DMSO-d₆) δ 6.76 (s, 1H), 7.03-7.34 (m, 5H), 7.37-7.56 (m, 5H, 7.64 (d, 1H), 7.96 (br s, 1H).

### EXAMPLE 94

### N′-((1-methyl-1H-imidazol-2-yl)methylidene)-3-phenyl-1H-indole-2-carbohydrazide

The desired product was prepared by substituting 1-methyl-1H-imidazole-2-carbaldehyde for 4-bromobenzaldehyde in Example 2. MS (ESI(+)) m/e 344 (M+H)⁺; ¹H NMR (300 MHz, DMSO-d₆) δ 3.95 (br d, 1H), 4.45 (br s, 1H), 7.02-7.66 (m, 9H), 8.05 (s, 1H), 8.75 (br s,1H).

### EXAMPLE 95

### N′-(1H-imidazol-5-ylmethylidene)-3-phenyl-1H-indole-2-carbohydrazide

The desired product was prepared by substituting 1H-imidazole-5-carbaldehyde for 4-bromobenzaldehyde in Example 2. MS (ESI(+)) m/e 330 (M+H)⁺; ¹H NMR (300 MHz, DMSO-d₆) δ 4.03 (s, 1H), 4.45 (br d, 1H), 7.05-7.57 (m, 9H), 8.75 (br s, 1H).

### EXAMPLE 96

### N′-((2-chloro-3-quinolinyl)methylidene)-3-phenyl-1H-indole-2-carbohydrazide

The desired product was prepared by substituting 2-chloro-3-quinolinecarbaldehyde for 4-bromobenzaldehyde in Example 2. MS (ESI(+)) m/e 425 (M+H)⁺; ¹H NMR (300 MHz, DMSO-d₆) δ 7.12-7.18 (m, 1H), 7.27-7.35 (m, 1H), 7.36-7.50 (br m, 2H), 7.51-7.60 (m, 3H), 7.65-7.73 (m, 2H), 7.82-7.89 (m, 1H), 7.93-8.00 (m, 1H), 8.21 (br s,1H), 8.55 (br s, 1H), 8.96 (br s, 1H).

### EXAMPLE 97

### 3-phenyl-N′-(1H-pyrrol-2-ylmethylidene)-1H-indole-2-carbohydrazide

The desired product was prepared by substituting 1H-pyrrole-2-carbaldehyde for 4-bromobenzaldehyde in Example 2. MS (ESI(+)) m/e 329 (M+H)⁺; ¹H NMR (300 MHz, DMSO-d₆) δ 6.12 (br d, 1H), 6.44 (br d, 1H), 6.90 (br d, 1H), 7.07-7.14 (m, 1H), 7.24-7.38 (m, 3H), 7.42-7.57 (m, 6H), 7.64 (d, 1H), 7.88 (s, 1H).

### EXAMPLE 98

### N′-((1-methyl-1H-pyrrol-2-yl)methylidene)-3-phenyl-1H-indole-2-carbohydrazide

The desired product was prepared by substituting 1-methyl-1H-pyrrole-2-carbaldehyde for 4-bromobenzaldehyde in Example 2. MS (ESI(+)) m/e 343 (M+H)⁺; ¹H NMR (300 MHz, DMSO-d₆) δ 3.84 (s, 3H), 6.08 (br d, 1H), 6.45 (br d, 1H), 6.94 (br d, H), 7.04-7.14 (m, 1H), 7.20-7.38 (m, 3H), 7.42-7.57 (m, 6H), 7.63 (d, 1H), 7.99 (s, 1H).

### EXAMPLE 99

### N′-((4-chloro-1-methyl-1H-pyrazol-3-yl)methylidene)-3-phenyl-1H-indole-2-carbohydrazide

The desired product was prepared by substituting 4-chloro-1-methyl-1H-pyrazole-3-carbaldehyde for 4-bromobenzaldehyde in Example 2. MS (ESI(+)) m/e 378 (M+H)⁺; ¹H NMR (300 MHz, DMSO-d₆) δ 3.63 (s, 3H), 7.09-7.57 (m, 9H), 7.63 (d, 1H), 8.02 (s, 1H).

### EXAMPLE 100

### N′-((4-(difluoromethoxy)phenyl)methylidene)-3-methyl-1H-indole-2-carbohydrazide

The desired product was prepared by substituting Example 61C and 4-(difluoromethoxy)benzaldehyde for Example 1C and 4-bromobenzaldehyde, respectively, in Example 2. MS (ESI(+)) m/e 344 (M+H)⁺; ¹H NMR (300 MHz, DMSO-d₆) δ 2.52 (s, 3H), 7.07 (t, 1H), 7.25 (t, 1H), 7.27 (d, 2H), 7.08-7.58 (t, 1H), 7.44 (d, 1H), 7.64 (d, 1H), 7.80 (d, 2H), 8.34 (s, 1H), 11.28 (s, 1H), 11.48 (s, 1H).

### EXAMPLE 101

### N′-((4-chlorophenyl)methylidene)-3-(phenylsulfonyl)-1H-indole-2-carbohydrazide

### EXAMPLE 101A

### Methyl 3-(phenylsulfanyl)-1H-indole-2-carboxylate

A mixture of 3-(phenylsulfonyl)-1H-indole-2-carboxylic acid (650 mg, 2.41 mmol, prepared according to the procedure described in Synthesis, 1988, 480) in benzene (20 mL) and methanol (5 mL) at room temperature was treated dropwise with 2M TMSCHN₂ in hexanes (3.6 mL, 7.23 mmol), stirred for 18 hours, quenched with acetic acid until gas evolution ceased, and concentrated. The concentrate was purified by flash column chromatography on silica gel with 4:1 hexanes/ethyl acetate to provide the desired product (595 mg, 87%). MS (CI) m/e 284 (M+H)⁺, 301 (M+NH₄)⁺.

### EXAMPLE 101B

### Methyl 3-(phenylsulfonyl)-1H-indole-2-carboxylate

A solution of Example 101A (390 mg, 1.38 mmol) in dichloromethane (15 mL) and methanol (10 mL) at 0° C. was treated portionwise with 70-75% 3-chloroperoxybenzoic acid (825 mg), warmed to room temperature, stirred for 8 hours, treated with 10% sodium bisulfite (20 mL), stirred for 5 minutes, and extracted with dichloromethane. The combined extracts were washed with saturated sodium bicarbonate and water, dried (MgSO₄), filtered, and concentrated. The concentrate was recrystallized from ethyl acetate/hexanes to provide the desired product (360 mg, 83%). MS (CI) m/e 316 (M+H)⁺, 333 (M+NH₄)⁺.

### EXAMPLE 101C

### 3-(phenylsulfonyl)-1H-indole-2-carbohydrazide

A suspension of Example 101B (340 mg, 1.08 mmol) in ethanol (8 mL) was treated with hydrazine hydrate (336 μL, 10.8 mmol), heated to reflux for 18 hours, cooled to room temperature, and cooled in a freezer for 2 hours. The precipitate by filtration and dried under vacuum to provide the desired product (277 mg, 81%). MS (CI) m/e 316 (M+H)⁺, 333 (M+NH₄)⁺.

### EXAMPLE 101D

### N′-((4-chlorophenyl)methylidene)-3-(phenylsulfonyl)-1H-indole-2-carbohydrazide

A solution of Example 101C (60 mg, 0.19 mmol) in ethanol (3 mL) was treated with 4-chlorobenzaldehyde (27 mg, 0.19 mmol), heated to reflux for 18 hours, cooled to room temperature, and cooled in a freezer for 2 hours. The precipitate was collected by filtration and dried under vacuum to provide the desired product as a mixture of cis- and trans-isomers (82 mg, 98%). MS (ESI) m/e 438, 440 (M+H)⁺; ¹H NMR (300 MHz, DMSO-d₆) δ 7.22-7.43 (m, 5H), 7.46-7.64 (m, 4H), 7.80-7.86 (m, 1.6H), 7.98 (m, 1.4H), 8.08 (m, 1H), 8.12 (s, 0.4H), 8.34 (s, 0.6H), 12.41 (s, 0.6H), 12.46 (s, 0.4H), 12.78 (s, 0.4H), 13.00 (s, 0.6H); Anal. calcd. for C₂₂H₁₆ClN₃O₃S: C, 60.34; H, 3.68; N, 9.60. Found: C, 60.15; H, 3.57; N, 9.50.

### EXAMPLE 102

### N′-((4-bromophenyl)methylidene)-3-(phenylsulfonyl)-1H-indole-2-carbohydrazide

The desired product was prepared as a mixture of cis- and trans-isomers by substituting 4-bromobenzaldehyde for 4-chlorobenzaldehyde in Example 101D. MS (ESI) m/e 482, 484 (M+H)⁺; ¹H NMR (300 MHz, DMSO-d₆) δ 7.22-7.44 (m, 4H), 7.47-7.64 (m, 4H), 7.68-7.85 (m, 3H), 7.98 (m, 1H), 8.05-8.14 (m, 1H), 8.12 (s, 04H), 8.32 (s, 1H), 12.42 (s, 0.6H), 12.47 (s, 0.4H), 12.77 (s, 0.4H), 13.00 (s, 0.6H); Anal. calcd. for C₂₂H₁₆BrN₃O₂S: C, 54.78; H, 3.34; N, 8.71. Found: C, 54.60; H, 3.23; N, 8.71.

### EXAMPLE 103

### 3-benzyl-N′-((4-chlorophenyl)methylidene)-1H-indole-2-carbohydrazide

### EXAMPLE 103A

### Ethyl 3-benzyl-1H-indole-2-carboxylate

Ethyl 2-oxo-4-phenylbutyrate (5 g, 24.3 mmol) at room temperature was treated with phenylhydrazine (2.38 mL, 24.3 mmol) and concentrated H₂SO₄ (3 drops), heated to 120° C. for 30 minutes, cooled to toom temperature, treated with ethanol (30 mL) and HCl gas, heated to 85-90° C. for 1 hour, and extracted with ethyl acetate. The combined extracts were washed with water, dried (Na₂SO₄), filtered, and concentrated. The concentrate was purified by recrystallization from ethanol/ethyl acetate to provide the desired product (4.57 g, 67%). MS (ESI) m/e 280 (M+H)⁺, 297 (M+NH₄)⁺, 278 (M−H)⁻.

### EXAMPLE 103B

### 3-benzyl-1H-indole-2-carbohydrazide

A solution of Example 103A (1.0 g, 3.58 mmol) in ethanol (10 mL) at room temperature was treated with hydrazine hydrate (1.11 mL, 35.8 mmol), heated to reflux for 4 hours, and concentrated. The precipitate was collected by filtration, washed with ethanol, and dried under vacuum to provide the desired product (750 mg, 79%). MS (ESI) m/e 266 (M+H)⁺, 288 (M+Na)⁺, 264 (M−H)⁻, 300 (M+Cl)⁻.

### EXAMPLE 103C

### 3-benzyl-N′-((4-chlorophenyl)methylidene)-1H-indole-2-carbohydrazide

A solution of Example 103B (100 mg, 0.38 mmol) in ethanol (10 mL) at room temperature was treated with 4-chlorobenzaldehyde (57.5 mg, 0.396 mmol), heated to reflux for 18 hours, cooled to room temperature, and filtered. The filter cake was washed with ethanol and dried under vacuum to provide the desired product (117 mg, 79%). mp 221-222° C.; MS (ESI) m/e 388, 390 (M+H)⁺, 386, 388 (M−H)⁻; ¹H NMR (DMSO-d₆) δ 4.44 (s, 2H), 7.00-7.31 (m, 7H), 7.44-7.47 (d, 1H), 7.52-7.55 (d, 2H), 7.58-7.61 (d, 1H), 7.76-7.78 (d, 2H), 8.33 (s, 1H), 11.41 (s, 1H), 11.71 (s, 1H); Anal. calcd. for C₂₃H₁₈ClN₃O: C, 71.22; H, 4.68; N, 10.83. Found: C, 71.03; H, 4.75; N, 10.74.

### EXAMPLE 104

### 3-benzyl-N′-((4-bromophenyl)methylidene)-1H-indole-2-carbohydrazide

A solution of Example 103B (100 mg, 0.38 mmol) in ethanol (10 mL) at room temperature was treated with 4-bromobenzaldehyde (73 mg, 0.396 mmol), heated to reflux for 18 hours, cooled to room temperature, and filtered. The filter cake was washed with ethanol and dried under vacuum to provide the desired product (138.8 mg, 84.5%). mp 227-229° C.; MS (ESI) m/e 432, 434 (M+H)⁺, 430, 432 (M−H)⁻; ¹H NMR (DMSO-d₆) δ 4.44 (s, 2H), 7.00-7.31 (m, 7H), 7.44-7.47 (d, 1H), 7.58-7.61 (d, 1H), 7.65-7.72 (m, 4H), 8.31 (s, 1H), 11.41 (s, 1H), 11.72 (s, 1H); Anal. calcd. for C₂₃H₁₈BrN₃O: C, 63.90; H, 4.20; N, 9.72. Found: C, 63.62; H, 4.19; N, 9.69.

### EXAMPLE 105

### 2-((2-(4-chlorobenzylidene)hydrazino)carbonyl)-N,N-diethyl-1H-indole-3-carboxamide

### EXAMPLE 105A

### Ethyl 3-formyl-1H-indole-2-carboxylate

A solution of POCl₃ (5.52 mL, 59.2 mmol) in DMF (18 mL, 232.5 mmol) at 0° C. was stirred for 40 minutes, treated dropwise with a solution of ethyl indole-2-carboxylate (10 g, 52.8 mmol) in DMF (15 mL), warmed to room temperature, stirred for 30 minutes, heated to 60° C. for 4 hours, cooled to room temperature, treated with water (60 mL), and adjusted to pH 7 with 2M NaOH. The precipitate was filtered, washed with water, dried under vacuum, and recrystallized from ethyl acetate to provide the desired product (10.9 g, 95%).

### EXAMPLE 105B

### 2-(ethoxycarbonyl)-1H-indole-3-carboxylic Acid

A solution of Example 105A (500 mg, 2.3 mmol) in tert-butanol (48 mL) and 2-methyl-2-butene (11.5 mL) at room temperature was treated with a solution of sodium chlorite (1.9 g, 21.1 mmol) and sodium dihydrogenphosphate (1.9 g, 15.9 mmol) in water (19 mL), stirred for 18 hours, and concentrated. The concentrate was diluted with water and extracted twice with hexanes. The aqueous phase was adjusted to pH 3 with 1N HCl and extracted three times with ethyl acetate. The combined ethyl acetate extracts were dried (Na₂SO₄), filtered, and concentrated to provide the desired product (530 mg, 99%). MS (ESI) m/e 234 (M+H)⁺, 251 (M+NH₄)⁺, 256 (M+Na)⁺, 232 (M−H)⁻.

### EXAMPLE 105C

### Ethyl 3-((diethylamino)carbonyl)-1H-indole-2-carboxylate

A solution of Example 105B (200 mg, 0.86 mmol) in DMF (10 mL) at room temperature was treated with diethylamine (106 μL, 1.03 mmol), EDC (181 mg, 0.94 mmol), HOBT (127.5 mg, 0.94 mmol), and 4-methylmorpholine, stirred for 18 hours, diluted with water, and extracted three times with ethyl acetate. The combined extracts were washed with water and brine, dried (Na₂SO₄), filtered, and concentrated. The concentrate was purified by flash column chromatography on silica gel with 50% ethyl acetate/hexanes to provide the desired product (188 mg, 76%). MS (ESI) m/e 289 (M+H)⁺, 311 (M+Na)⁺, 287 (M−H)⁻.

### EXAMPLE 105D

### N,N-diethyl-2-(hydrazinocarbonyl)-1H-indole-3-carboxamide

A solution of Example 105C (188 mg, 0.65 mmol) in ethanol (10 mL) was treated with hydrazine hydrate (202 μL, 6.5 mmol), heated to reflux for 18 hours, and concentrated. The concentrate was purified by flash column chromatography on silica gel with ethyl acetate to provide the desired product (148 mg, 83%). MS (ESI) m/e 275 (M+H)⁺, 297 (M+Na)⁺, 273 (M−H)⁻, 309 (M+Cl)⁻.

### EXAMPLE 105E

### 2-((2-(4-chlorobenzylidene)hydrazino)carbonyl)-N,N-diethyl-1H-indole-3-carboxamide

A solution of Example 105D (148 mg, 0.54 mmol) in ethanol (8 mL) at room temperature was treated with 4-chlorobenzaldehyde (82 mg, 0.567 mmol), heated to reflux for 18 hours, cooled to room temperature, and filtered. The filter cake was washed with ethanol and dried under vacuum to provide the desired product (138 mg, 64%). mp 255-257° C.; MS (ESI) m/e 397, 399 (M+H)⁺, 395, 397 (M−H)⁻, ¹H NMR (DMSO-d₆) δ 0.80-1.40 (m, 6H), 7.16-7.21 (t, 1H), 7.29-7.34 (t, 1H), 7.48-7.55 (m, 4H), 7.80-7.83 (d, 2H), 8.27 (s, 1H), 12.00-12-30 (m, 2H); Anal. calcd. for C₂₁H₂₁ClN₄O₂: C, 63.55; H, 5.33; N, 14.12. Found: C, 63.42; H, 5.24; N, 14.02.

### EXAMPLE 106

### 2-((2-(4-chlorobenzylidene)hydrazino)carbonyl)-N,N-dimethyl-1H-indole-3-carboxamide

### EXAMPLE 106A

### 2-(hydrazinocarbonyl)-N,N-dimethyl-1H-indole-3-carboxamide

The desired product was prepared by substituting dimethylamine for diethylamine in Examples 105C and 105D.

### EXAMPLE 106B

### 2-((2-(4-chlorobenzylidene)hydrazino)carbonyl)-N,N-dimethyl-1H-indole-3-carboxamide

A solution of Example 106A (42 mg, 0.17 mmol) in ethanol (5 mL) was treated with 4-chlorobenzaldehyde (26 mg, 0.18 mmol), heated to reflux for 18 hours, cooled to room temperature, and concentrated. The concentrate was purified by flash column chromatography on silica gel with 30% ethyl acetate/hexanes to provide the desired product (40.3 mg, 64%). MS (ESI) m/e 369, 371 (M+H)⁺, 395, 397 (M−H)⁻; ¹H NMR (DMSO-d₆) δ 2.80-3.22 (m, 6H), 7.18-7.23 (t, 1H), 7.29-7.34 (t, 1H), 7.52-7.57 (m, 4H), 7.81-7.84 (d, 2H), 8.29 (s, 1H); Anal. calcd. for C₁₉H₁₇ClN₄O₂ 0.55H₂O: C, 60.26; H, 4.82; N, 14.33. Found: C, 60.52; H, 4.49; N, 14.79.

### EXAMPLE 107

### 2-((2-(4-chlorobenzylidene)hydrazino)carbonyl)-N-phenyl-1H-indole-3-carboxamide

The desired product was prepared by substituting aniline for ethylamine in Example 105. mp>260° C.; MS (ESI) m/e 417, 419 (M+H)⁺, 415, 417 (M−H)⁻, ¹H NMR (DMSO-d₆) δ 7.13-7.18 (t, 1H), 7.25-7.30 (t, 1H), 7.35-7.43 (m, 4H), 7.53-7.56 (d, 2H), 7.59-7.62 (d, 2H), 7.76-7.79 (d, 2H), 7.83-7.86 (d, 2H), 7.99-8.02 (d, 1H), 8.35 (s, 1H), 10.83 (s, 1H), 12.58 (s, 1H), 13.26 (s, 1H); Anal. calcd. for C₂₃H₁₇ClN₄O₂: C, 66.27; H, 4.11; N, 13.44. Found: C, 65.97; H, 3.81; N, 13.21.

### EXAMPLE 108

### N′-((4-chlorophenyl)methylidene)-3-(methylthio)-1H-indole-2-carbohydrazide

### EXAMPLE 108A

### Ethyl 3-(methylsulfanyl)-2-oxopropanoate

A solution of methyl disulfide (5.0 g, 53 mmol) in hexanes (100 mL) at 0° C. was treated with 1.4M methyllithium in diethyl ether (36 mL, 50 mmol), stirred for 30 minutes, cooled to −78° C., treated with ethyl bromopyruvate (8.86 g, 45.4 mmol), stirred for 80 minutes, and poured into saturated ammonium chloride. The aqueous phase was extracted with diethyl ether and the combined extracts were dried (Na₂SO₄), filtered, and concentrated to provide the desired product (6.8 g). MS (DCI) m/e 180 (M+NH₄)⁺.

### EXAMPLE 108B

### Ethyl 3-(methylsulfanyl)-1H-indole-2-carboxylate

A mixture of Example 108A (6.8 g), phenylhydrazine (4.9 g, 45.4 mmol), and concentrated sulfuric acid (5 drops) was heated to 120° C. for 15 minutes, cooled to room temperature, treated with ethanol (70 mL), treated with HCl gas for 2 minutes, heated to 85-90° C. for 1 hour, poured into water, and extracted with ethyl acetate. The combined extracts were washed with water and brine, dried (Na₂SO₄), filtered, and concentrated. The concentrate was purified by flash column chromatography on silica gel with 10% to 20% ethyl acetate/hexanes to provide a solid which was washed with hexanes and dried to provide the desired product (2.07 g, 19%) MS (ESI) m/e 236 (M+H)⁺, 253 (M+NH₄)⁺, 234 (M−H)⁻.

### EXAMPLE 108C

### 3-(methylsulfanyl)-1H-indole-2-carbohydrazide

The desired product was prepared by substituting Example 108B for Example 103A in Example 103B.

### EXAMPLE 108D

### N′-((4-chlorophenyl)methylidene)-3-(methylthio)-1H-indole-2-carbohydrazide

The desired product was prepared by substituting Example 108C for Example 103B in Example 103C. mp 232-235° C. MS (ESI) m/e 344, 346 (M+H)⁺, 342, 344 (M−H)⁻, ¹H NMR (DMSO-d₆) δ 2.42 (s, 3H), 7.17-7.22 (t, 1H), 7.28-7.33 (t, 1H), 7.49-7.52 (d, 1H), 7.54-7.57 (d, 2H), 7.73-7.75 (d, 1H), 7.80-7.83 (d, 2H), 8.47 (s, 1H), 11.83 (s, 1H), 12.16 (s, 1H);

Anal. calcd. for C₁₇H₁₄ClN₃OS: C, 59.39; H, 4.10; N, 12.22. Found: C, 59.07; H, 4.14; N, 12.05.

### EXAMPLE 109

### 3-(methylthio)-N′-(1,3-thiazol-2-ylmethylidene)-1H-indole-2-carbohydrazide

The desired product was prepared by substituting Example 108C and 1,3-thiazole-2-carbaldehyde for Example 103B and 4-chlorobenzaldehyde, respectively, in Example 103C.

mp 216-222° C.; ¹H NMR (DMSO-d₆) δ 2.40 (s, 3H), 7.17-7.23 (t, 1H), 7.29-7.34 (t, 1H), 7.49-7.52 (d, 1H), 7.74-7.76 (d, 1H), 7.89 (1H), 8.00-8.01 (d, 1H), 8.71 (s, 1H), 12.07 (s, 1H), 12.17 (br s, 1H); MS (ESI) m/e 317 (M+H)⁺, 339 (M+Na)⁺, 315 (M−H)⁻, 351 (M+Cl)⁻; Anal. calcd. for C₁₄H₁₂N₄OS₂: C, 53.15; H, 3.82; N, 17.71. Found: C, 52.87; H, 3.76; N, 17.44.

### EXAMPLE 110

### N′-((4-chlorophenyl)methylidene)-3-(methylsulfonyl)-1H-indole-2-carbohydrazide

### EXAMPLE 110A

### 3-(methylsulfonyl)-1H-indole-2-carbohydrazide

A solution of Example 108B (500 mg, 2.13 mmol) in dichloromethane (25 mL) and methanol (17 mL) at 0° C. was treated with mCPBA (1.27 g, 5.32 mmol), warmed to room temperature, stirred at room temperature for 5 hours, treated with saturated sodium bicarbonate, and extracted three times with dichloromethane. The combined extracts were washed sequentially with saturated sodium bicarbonate, water, and brine, dried (Na₂SO₄), filtered, and concentrated. The concentrate was triturated with ethyl acetate/hexanes and filtered to provide the desired product (461 mg, 81%). MS (ESI) m/e 268 (M+H)⁺, 285 (M+NH₄)⁺, 266 (M−H)⁻.

### EXAMPLE 110B

### N′-((4-chlorophenyl)methylidene)-3-(methylsulfonyl)-1H-indole-2-carbohydrazide

The desired product was prepared by substituting Example 110A for Example 103B in Example 103C. mp>280° C.; MS (ESI) m/e 376, 378 (M+H)⁺, 374, 376 (M−H)⁻; ¹H NMR (DMSO-d₆) δ 3.14 (s, 1H), 3.36 (s, 2H), 7.27-7.47 (m, 4H), 7.55-7.59 (m, 2H), 7.80-7.83 (d, 1H), 7.89-7.91 (d, 0.33H), 7.97-7.99 (d, 0.67H), 8.14 (s, 0.33H), 8.32 (s, 0.67H), 12.33 (s, 0.67H), 12.40 (s, 0.33H), 12.73 (s, 0.33H), 12.97 (s, 0.67H); Anal. calcd. for C₁₇H₁₄N₃O₃ClS: C, 54.33; H, 3.75; N, 11.18. Found: C, 54.15; H, 3.63; N, 11.16.

### EXAMPLE 111

### 3-(methylsulfonyl)-N′-(1,3-thiazol-2-ylmethylidene)-1H-indole-2-carbohydrazide

The desired product was prepared by substituting Example 110A and 1,3-thiazole-2-carbaldehyde for Example 103B and 4-chlorobenzaldehyde, respectively, in Example 103C.

mp>280° C.; MS (ESI) m/e 349 (M+H)⁺, 366 (M+NH₄)⁺, 371 (M+Na)⁺, 347 (M−H)⁻, 383 (M+Cl)⁻; ¹H NMR (DMSO-d₆) δ 3.20 (s, 1H), 3.37 (s, 2H), 7.29-7.42 (m, 2H), 7.54-7.61(m, 2H), 7.69-7.71 (d, 0.33H), 7.89-7.91 (d, 0.67H), 7.93-8.02 (m, 2H), 8.32 (s, 0.33H), 8.52 (s, 0.67H), 12.58-12.63 (m, 1H), 12.75 (s, 0.33H), 13.02 (s, 0.67H); Anal. calcd. for C₁₄H₁₂N₄O₃S₂: C, 48.26; H, 3.47; N, 16.08. Found: C, 48.05; H, 3.30; N, 16.06.

### EXAMPLE 112

### 7-chloro-N′-((4-chlorophenyl)methylidene)-3-methyl-1H-indole-2-carbohydrazide

### EXAMPLE 112A

### Ethyl 7-chloro-3-methyl-1H-indole-2-carboxylate

A mixture of 2-chlorophenylhydrazine hydrochloride (2.75 g, 15.38 mmol) and 2-oxo-butyric acid ethyl ester (2.0 g, 15.38 mmol) in ethanol (40 mL) was treated with concentrated H₂SO₄ (6 drops), heated to reflux for 2 hours, cooled to room temperature, treated with HCl gas for about 3 minutes, heated to reflux for 90 minutes, cooled to room temperature, diluted with water, and extracted with ethyl acetate. The combined extracts were washed with water and brine, dried (MgSO₄), filtered, and concentrated. The concentrate was recrystallized from ethanol to provide the desired product (1.56 g, 43%). MS (CI) m/e 238 (M+H)⁺.

### EXAMPLE 112B

### 7-chloro-3-methyl-1H-indole-2-carbohydrazide

The desired product was prepared by substituting Example 112A for Example 101B in 101C. MS (CI) m/e 224 (M+H)⁺.

### EXAMPLE 112C

### 7-chloro-N′-((4-chlorophenyl)methylidene)-3-methyl-1H-indole-2-carbohydrazide

The desired product was prepared by substituting Example 112B for Example 101C in Example 101D. MS (CI) m/e 346, 348 (M+H)⁺; ¹H NMR (300 MHz, DMSO-d₆) δ 2.55 (s, 3H), 7.11 (t, 1H), 7.37 (d, 1H), 7.55 (d, 2H), 7.65 (d, 1H), 7.79 (d, 2H), 8.36 (s, 1H), 11.44 (s, 1H), 11.75 (s, 1H); Anal. calcd. for C₁₇H₁₃C₁₂N₃O: C, 58.98; H, 3.78; N, 12.14. Found: C, 58.87; H, 3.69; N, 12.15.

### EXAMPLE 113

### 7-chloro-3-methyl-N′-(1,3-thiazol-2-ylmethylidene)-1H-indole-2-carbohydrazide

The desired product was prepared by substituting Example 112B and 1,3-thiazole-2-carbaldehyde for Example 101C and 4-chlorobenzaldehyde, respectively, in Example 101D. MS (CI) 319, 321 (M+H)⁺, 336, 338 (M+NH₄)⁺; ¹H NMR (300 MHz, DMSO-d₆) δ 2.54 (s, 3H), 7.12 (t, 1H), 7.38 (d, 1H), 7.66 (d, 1H), 7.88 (d, 1H), 7.99 (d, 1H), 8.57 (s, 1H), 11.46 (br s, 1H), 12.01 (br s, 1H); Anal. calcd. for C₁₄H₁₁ClN₄OS: C, 52.75; H, 3.48; N, 17.58. Found: C, 52.70; H, 3.44; N, 17.57.

### EXAMPLE 114

### 7-chloro-N′-((4-methoxyphenyl)methylidene)-3-methyl-1H-indole-2-carbohydrazide

The desired product was prepared by substituting Example 112B and 4-methoxybenzaldehyde for Example 101C and 4-chlorobenzaldehyde, respectively, in Example 101D. MS (CI) 342 (M+H)⁺; ¹H NMR (300 MHz, DMSO-d₆) δ 2.55 (s, 3H), 3.82 (s, 3H), 7.05 (d, 2H), 7.10 (t, 1H), 7.36 (d, 1H), 7.64 (d, 1H), 7.71(d, 2H), 8.32 (s, 1H), 11.43 (s, 1H), 11.57 (s, 1H); Anal. calcd. for C₁₈H₁₆ClN₃O₂: C, 63.25; H, 4.72; N, 12.29. Found: C, 63.13; H, 4.66; N, 12.33.

### EXAMPLE 115

### 3-(methylsulfonyl)-N′-(2-naphthylmethylidene)-1H-indole-2-carbohydrazide

The desired product was prepared by substituting Example 110A and 2-naphthaldehyde for Example 103B and 4-chlorobenzaldehyde, respectively, in Example 103C. mp>250° C.;

¹H NMR (DMSO-d₆) δ 3.17 (s, 1H), 3.38 (s, 2H), 7.29-7.61 (m, 5.33H), 7.77-8.04 (m, 5H), 8.24 (s, 0.67H), 8.31 (s, 0.33H), 8.48 (s, 0.67H), 12.36 (s, 0.67H), 12.42 (s, 0.33H), 12.76 (s, 0.33H), 12.98 (s, 0.67H); Anal. calcd. for C₂₁H₁₇N₄O₃S: C, 64.44; H, 4.38; N, 10.73. Found: C64.28; H, 4.30; N, 10.83.

### EXAMPLE 116

### N′-((4-chlorophenyl)methylidene)-6-methoxy-3-methyl-1H-indole-2-carbohydrazide

### EXAMPLE 116A

### 6-methoxy-3-methyl-1H-indole-2-carboxylic Acid

The desired product was prepared according to the procedure descirbed in J. Org. Chem. 1997, 62, 9298.

### EXAMPLE 116B

### Tert-butyl 2-((6-methoxy-3-methyl-1H-indol-2-yl)carbonyl)hydrazinecarboxylate

A mixture of Example 116A (670 mg, 3.27 mmol) and tert-butyl carbazate (432 mg, 3.27 mmol) in DMF (15 mL) at room temperature was treated with 1-hydroxybenzotriazole (486 mg, 3.60 mmol), 4-methylmorpholine (540 μL, 4.90 mmol), and EDC.HCl (689 mg, 3.60 mmol), stirred for 4 hours, diluted with water, and extracted with ethyl acetate. The combined extracts were washed twice with water and brine, dried (MgSO₄), filtered, and concentrated to provide the desired product.

### EXAMPLE 116C

### 6-methoxy-3-methyl-1H-indole-2-carbohydrazide

A suspension of Example 116B (3.27 mmol) in dichloromethane (15 mL) at 0° C. was treated with TFA (15 mL), stirred for 15 minutes, warmed to room temperature, stirred for 1 hour, and concentrated. The concentrate was treated dropwise with saturated sodium bicarbonate until gas evolution ceased and filtered. The filter cake was washed with water and dried under vacuum at 60° C. to provide the desired product (617 mg, 86%). MS (CI) m/e 220 (M+H)⁺.

### EXAMPLE 116D

### N′-((4-chlorophenyl)methylidene)-6-methoxy-3-methyl-1H-indole-2-carbohydrazide

The desired product was prepared by substituting Example 116C for Example 101C in Example 101D. HRMS (ESI) calcd. for C₁₈H₁₇N₃O₂Cl (M+H)⁺: 342.1009. Found: 342.1007; ¹H NMR (300 MHz, DMSO-d₆) δ 2.50 (s, 3H), 3.80 (s, 3H), 6.73 (d, 1H), 6.88 (s, 1H), 7.50-7.70 (m, 3H), 7.77 (d, 2H), 8.33 (s, 1H), 11.13 (s, 1H), 11.41 (s, 1H).

### EXAMPLE 117

### 4,6-dichloro-N′-((4-chlorophenyl)methylidene)-3-methyl-1H-indole-2-carbohydrazide

### EXAMPLE 117A

### Ethyl 4,6-dichloro-3-methyl-1H-indole-2-carboxylate

The desired product was prepared by substituting 3,5-dichlorophenylhydrazine hydrochloride for 2-chlorophenylhydrazine hydrochloride in Example 112A. MS (CI) m/e 271 (M+H)⁺.

### EXAMPLE 117B

### 4,6-dichloro-3-methyl-1H-indole-2-carboxylic Acid

A suspension of Example 117A (1.00 g, 3.69 mmol) and KOH (620 mg, 11.07 mmol) in ethanol (16 mL) and water (8 mL) was heated to 90° C., stirred for 1.5 hours, cooled to room temperature, adjusted to pH 3 with 4N HCl, and extracted with ethyl acetate. The combined extracts were washed with brine, dried (MgSO₄), filtered, and concentrated to provide the desired product (0.88 g, 98%). MS (CI) m/e 243 (M)⁺.

### EXAMPLE 117C

### 7-chloro-3-methyl-1H-indole-2-carbohydrazide

The desired product was prepared by substituting Example 117B for Example 116B in Example 116C. MS (CI) m/e 258 (M+H)⁺.

### EXAMPLE 117D

### 4,6-dichloro-N′-((4-chlorophenyl)methylidene)-3-methyl-1H-indole-2-carbohydrazide

The desired product was prepared by substituting Example 117C for Example 101C in Example 101D. MS (CI) 380 (M+H)⁺; ¹H NMR (300 MHz, DMSO-d₆) δ 2.69 (s, 3H), 7.16 (d, 1H), 7.44 (d, 1H), 7.53 (d, 2H), 7.75 (br s, 2H), 8.35 (br s, 1H), 11.85 (br s, 2H); Anal. calcd. for C₁₇H₁₂Cl₃N₃O: C, 53.64; H, 3.18; N, 11.04. Found: C, 53.49; H, 3.05; N, 10.99.

### EXAMPLE 118

### N′-((4-chlorophenyl)methylidene)-3-methoxy-1H-indole-2-carbohydrazide

### EXAMPLE 118A

### Methyl 2-((2-ethoxy-2-oxoethyl)amino)benzoate

A mixture of methyl anthranilate (2.0 g, 13.2 mmol) and ethyl bromoacetate (734 μL, 6.6 mmol) at between 140 and 150° C. was stirred for 1 hour, diluted with water, and extracted three times with ethyl acetate. The combined extracts were washed with brine, dried (Na₂SO₄), filtered, and concentrated. The concentrate was purified by flash column chromatography on silica gel with 5% ethyl acetate/hexanes to provide the desired product (936.7 mg, 60%). MS (ESI) m/e 238 (M+H)⁺.

### EXAMPLE 118B

### Ethyl 3-hydroxy-1H-indole-2-carboxylate

A boiling solution of Example 118A (912.5 mg, 3.85 mmol) in ethanol was rapidly treated with a solution of sodium (159 mg, 6.9 mmol) in ethanol (2 mL), heated to reflux for 1 hour, diluted with water, and extracted with three portions of diethyl ether. The aqueous layer was adjusted to pH 7 with gaseous carbon dioxide and filtered. The filter cake was triturated with 30% ethanol to provide the desired product (289 mg, 36.7%). MS (ESI) m/e 204 (M−H)⁻.

### EXAMPLE 118C

### Ethyl 3-methoxy-1H-indole-2-carboxylate

A solution of Example 118B (289 mg, 1.42 mmol) and potassium hydroxide (95 mg, 0.7 mmol) in water (2 mL) at room temperature was treated with dimethylsulfate (217 μL, 2.30 mmol), stirred for 5 hours, diluted with water, and extracted with ethyl acetate. The combined extracts were dried over sodium sulfate, filtered, and concentrated. The concentrate was purified by flash column chromatography on silica gel with 10% ethyl acetate/hexanes. The product was recrystallized from hexanes to provide the desired product (138 mg, 44%). MS (ESI) m/e 220 (M+H)⁺, 218 (M−H)⁻.

### EXAMPLE 118D

### 3-methoxy-1H-indole-2-carbohydrazide

The desired product was prepared by substituting Example 118C for Example 104A in Example 104B.

### EXAMPLE 118E

### N′-((4-chlorophenyl)methylidene)-3-methoxy-1H-indole-2-carbohydrazide

The desired product was prepared by substituting Example 118D for Example 103B in Example 103C. mp 249-251° C.; MS (ESI) m/e 328, 330 (M+H)⁺, 326, 328 (M−H)⁻; ¹H NMR (DMSO-d₆) δ 4.21 (s, 3H), 7.02-7.07 (t, 1H), 7.21-7.26 (t, 1H), 7.39-7.41 (d, 1H), 7.53-7.56 (d, 2H), 7.75-7.78 (d, 2H), 7.82-7.85 (d, 1H), 8.47 (s, 1H), 10.81 (s, 1H), 11.39 (s, 1H); Anal. calcd. for C₁₇H₁₄N₃O₂Cl: C, 62.30; H, 4.31; N, 12.82. Found: C, 62.22; H, 4.28; N, 12.69.

### EXAMPLE 119

### 3-bromo-N′-((4-chlorophenyl)methylidene)-1H-indole-2-carbohydrazide

### EXAMPLE 119A

### Ethyl 3-bromo-1H-indole-2-carboxylate

A solution of ethyl indole-2-carboxylate (1.0 g, 5.29 mmol) in pyridine (23 mL) and water (2 mL) at 0° C. was treated with a solution of pyridinium bromide perbromide (1.7 8 g, 5.55 mmol) in pyridine (30 mL), treated with ice water, and extracted three times with diethyl ether. The combined extracts were washed with 1N HCl, dried (Na₂SO₄), filtered, and concentrated to provide the desired product (1.28 g, 90%). MS (ESI), m/e 266, 268 (M−H)⁻.

### EXAMPLE 119B

### 3-bromo-1H-indole-2-carbohydrazide

The desired product was prepared by substituting Example 119A for Example 103A in Example 103B.

### EXAMPLE 119C

### 3-bromo-N′-((4-chlorophenyl)methylidene)-1H-indole-2-carbohydrazide

The desired product was prepared by substituting Example 119B for Example 103B in Example 103C. mp>250 C; MS (ESI) m/e 377 (M+H)⁺; ¹H NMR (DMSO-d₆) δ 7.18-7.24 (t, 1H), 7.31-7.36 (t, 1H), 7.49-7.55 (m, 4H), 7.76-7.78 (m, 2H), 7.75-7.78 (d, 2H), 8.39 (s, 1H), 11.68 (s, 1H); Anal. calcd. for C₁₆H₁₁N₃O₂ClBr: C, 51.02; H, 2.94; N, 11.16. Found: C, 51.08; H, 2.96; N, 11.31.

### EXAMPLE 120

### 4,6-dichloro-N′-((4-cyanophenyl)methylidene)-3-methyl-1H-indole-2-carbohydrazide

The desired product was prepared by substituting Example 116C and 4-formylbenzonitrile for Example 101C and 4-chlorobenzaldehyde, respectively, in Example 101D. MS (CI) m/e 371, 373 (M+H)⁺; ¹H NMR (300 MHz, DMSO-d₆) δ 2.69 (s, 3H), 7.17 (d, 1H), 7.44 (d, 1H), 7.92 (s, 4H), 8.39 (br s, 1H), 11.80-12.00 (br m, 2H); Anal. calcd. for C₁₈H₁₂C₁₂N₄O: C, 58.24; H, 3.26; N, 15.09. Found: C, 57.86; H, 3.31; N, 15.02.

### EXAMPLE 121

### 6-chloro-N′-((4-chlorophenyl)methylidene)-3-methyl-1H-indole-2-carbohydrazide

### EXAMPLE 121A

### Ethyl 6-chloro-3-methyl-1H-indole-2-carboxylate

The desired product was prepared as a 4:1 mixture with Example 124A by substituting 3-chlorophenylhydrazine hydrochloride for 2-chlorophenylhydrazine hydrochloride in Example 112A. MS (CI) m/e 238 (M+H)⁺.

### EXAMPLE 121B

### 6-chloro-3-methyl-1H-indole-2-carboxylic Acid

The desired product was prepared by substituting Example 121A for Example 117A in Example 117B. MS (CI) m/e 209 (M)⁺.

### EXAMPLE 121C

### 6-chloro-3-methyl-1H-indole-2-carbohydrazide

The desired product was prepared by substituting Example 121B for Example 116A in Examples 116B and 116C. MS (CI) m/e 224 (M+H)⁺.

### EXAMPLE 121D

### 6-chloro-N′-((4-chlorophenyl)methylidene)-3-methyl-1H-indole-2-carbohydrazide

The desired product was prepared by substituting Example 121C for Example 101C in Example 101D. MS (CI) m/e 346, 348 (M+H)⁺; ¹H NMR (300 MHz, DMSO-d₆) δ 2.51 (s, 3H), 7.09 (dd, 1H), 7.47 (s, 1H), 7.54 (d, 2H), 7.67 (d, 1H), 7.76 (d, 2H), 8.35 (br s, 1H), 11.47 (br s, 1H), 11.58 (br s, 1H); Anal. calcd. for C₁₇H₁₃Cl₂N₃O: C, 58.98; H, 3.78, N, 12.14. Found: C, 59.03; H, 3.56; N, 12.10.

### EXAMPLE 122

### 6-chloro-N′-((4-methoxyphenyl)methylidene)-3-methyl-1H-indole-2-carbohydrazide

The desired product was prepared by substituting Example 121C and 4-methoxybenzaldehyde for Example 101C and 4-chlorobenzaldehyde, respectively, in Example 101D; MS (CI) m/e 342 (M+H)⁺; ¹H NMR (300 MHz, DMSO-d₆) δ 2.51 (s, 3H), 3.82 (s, 3H), 7.03 (d, 2H), 7.08 (dd, 1H), 7.46 (s, 1H), 7.67 (m, 3H), 8.30 (s, 1H), 11.37 (br s, 1H), 11.44 (br s, 1H); Anal. cacld. for C₁₈H₁₆ClN₃O₂: C, 63.25; H, 4.72; N, 12.29. Found: C, 63.10; H, 4.55; N, 12.22.

### EXAMPLE 123

### 6-chloro-N′-((4-cyanophenyl)methylidene)-3-methyl-1H-indole-2-carbohydrazide

The desired product was prepared by substituting Example 121C and 4-formylbenzonitrile for Example 101C and 4-chlorobenzaldehyde, respectively, in Example 101D. MS (CI) m/e 337, 339 (M+H)⁺; ¹H NMR (300 MHz, DMSO-d₆) δ 2.51 (s, 3H), 7.09 (d, 1H), 7.48 (s, 1H), 7.68 (s, 1H), 7.68 (d, 1H), 7.92 (s, 4H), 8.40 (s, 1H), 11.49 (br s, 1H), 11.76 (br s, 1H); Anal. calcd. for C₁₈H₁₃ClN₄O: C, 64.19; H, 3.89; N, 16.64. Found: C, 63.95; H, 3.63; N, 16.47.

### EXAMPLE 124

### 4-chloro-N′-((4-chlorophenyl)methylidene)-3-methyl-1H-indole-2-carbohydrazide

### EXAMPLE 124A

### Ethyl 4-chloro-3-methyl-1H-indole-2-carboxylate

The desired product was prepared as described in Example 121A. MS (CI) m/e 238 (M+H)⁺.

### EXAMPLE 124B

### 4-chloro-3-methyl-1H-indole-2-carboxylic Acid

The desired product was prepared by substituting Example 124A for Example 117A in Example 117B. MS (CI) m/e 210 (M+H)⁺.

### EXAMPLE 124C

### 4-chloro-3-methyl-1H-indole-2-carbohydrazide

The desired product was prepared by substituting Example 124B for Example 116A in Examples 116B and 116C. MS (CI) m/e 224 (M+H)⁺.

### EXAMPLE 124D

### 4-chloro-N′-((4-chlorophenyl)methylidene)-3-methyl-1H-indole-2-carbohydrazide

The desired product was prepared by substituting Example 124C for Example 101C in Example 101D. MS (CI) m/e 346, 348 (M+H)⁺; ¹H NMR (300 MHz, DMSO-d₆) δ 2.72 (s, 3H), 7.07 (d, 1H), 7.18 (t, 1H), 7.39 (d, 1H), 7.53 (d, 2H), 7.76 (br d, 2H), 8.34 (br s, 1H), 11.72 (br s, 2H); Anal. calcd. For C₁₇H₁₃Cl₂N₃O: C, 58.98; H, 3.78; N, 12.14. Found: C, 58.98; H, 3.88; N, 12.09.

### EXAMPLE 125

### 4-chloro-N′-((4-methoxyphenyl)methylidene)-3-methyl-1H-indole-2-carbohydrazide

The desired product was prepared by substituting Example 124C and 4-methoxybenzaldehyde for Example 101C and 4-chlorobenzaldehyde, respectively, in Example 101D. MS (CI) 342, 344 (M+H)⁺; ¹H NMR (300 MHz, DMSO-d₆) δ 2.72 (s, 3H), 3.82 (s, 3H), 7.05 (m, 3H), 7.17 (t, 1H), 7.39 (d, 1H), 7.68 (br d, 2H), 8.30 (br s, 1H), 11.51 (br s, 1H), 11.68 (br s, 1H); Anal. calcd. for C₁₈H₁₆ClN₃O₂: C, 63.25; H, 4.72; N, 12.29. Found: C, 63.15; H, 4.86; N, 12.27.

### EXAMPLE 126

### 4-chloro-N′-((4-cyanophenyl)methylidene)-3-methyl-1H-indole-2-carbohydrazide

The desired product was prepared by substituting Example 124C and 4-formylbenzonitrile for Example 101C and 4-chlorobenzaldehyde, respectively, in Example 101D. MS (CI) m/e 337, 339 (M+H)⁺; ¹H NMR (300 MHz, DMSO-d₆) δ 2.72 (s, 3H), 7.07 (d, 1H), 7.19 (t, 1H), 7.40 (d, 1H), 7.92 (s, 4H), 8.39 (s, 1H), 11.72 (s, 1H), 11.90 (s, 1H); Anal. calcd. for C₁₈H₁₃ClN₄O: C, 64.19; H, 3.89; N, 16.64. Found: C, 63.99; H, 3.88; N, 16.58.

### EXAMPLE 127

### N′-((4-bromophenyl)methylidene)-6-methoxy-3-methyl-1H-indole-2-carbohydrazide

The desired product was prepared by substituting Example 116C and 4-bromobenzaldehyde for Example 101C and 4-chlorobenzaldehyde, respectively, in Example 101D. MS (CI) m/e 386, 388 (M+H)⁺; ¹H NMR (300 MHz, DMSO-d₆) δ 2.50 (s, 3H), 3.80 (s, 3H), 6.73 (dd, 1H), 6.88 (d, 1H), 7.51 (d, 1H), 7.64-7.72 (m, 4H), 8.31 (s, 1H), 11.11 (s, 1H), 11.40 (s, 1H). Anal. calcd. for C₁₈H₁₆BrN₃O₂: C, 55.97; H, 4.18; N, 10.88. Found: C, 56.03; H, 4.14; N, 10.74.

### EXAMPLE 128

### 6-methoxy-N′-((4-methoxyphenyl)methylidene)-3-methyl-1H-indole-2-carbohydrazide

The desired product was prepared by substituting Example 116C and 4-methoxybenzaldehyde for Example 101C and 4-chlorobenzaldehyde, respectively, in Example 101D. MS (CI) m/e 338 (M+H)⁺; ¹H NMR (300 MHz, DMSO-d₆) δ 2.50 (s, 3H), 3.79 (s, 3H), 3.82 (s, 3H), 6.72 (dd, 1H), 6.88 (s, 1H), 7.03 (d, 2H), 7.50 (d, 1H), 7.68 (d, 2H), 8.28 (s, 1H), 11.08 (s, 1H), 11.19 (s, 1H); Anal. calcd. for C₁₉H₁₉N₃O₃: C, 67.64; H, 5.68; N, 12.46. Found: C, 67.69; H, 5.65; N, 12.44.

### EXAMPLE 129

### N′-((4-bromophenyl)methylidene)-4,6-dichloro-3-methyl-1H-indole-2-carbohydrazide

The desired product was prepared by substituting Example 117C and 4-bromobenzaldehyde for Example 101C and 4-chlorobenzaldehyde, respectively, in Example 101D. MS (CI) m/e 424, 426, 428 (M+H)⁺; ¹H NMR (300 MHz, DMSO-d₆) δ 2.65 (s, 3H), 7.16 (d, 1H), 7.44 (d, 1H), 7.68 (s, 4H), 8.33 (s, 1H), 11.75-11.95 (br m, 2H); Anal. calcd. for C₁₇H₁₂BrCl₂N₃O: C, 48.03; H, 2.85; N, 9.88. Found: C, 48.06; H, 2.93; N, 9.81.

### EXAMPLE 130

### 4,6-dichloro-3-methyl-N′-(pyridin-3-ylmethylidene)-1H-indole-2-carbohydrazide

The desired product was prepared by substituting Example 117C and nicotinaldehyde for Example 101C and 4-chlorobenzaldehyde, respectively, in Example 101D. MS (CI) m/e 347, 349 (M+H)⁺; ¹H NMR (300 MHz, DMSO-d₆) δ 2.70 (s, 1H), 7.16 (s, 1H), 7.44 (s, 1H), 7.50 (m, 1H), 8.14 (m, 1H), 8.40 (m, 1H), 8.62 (d, 1H), 8.87 (s, 1H), 11.88 (br s, 2H); Anal. calcd. for C₁₆H₁₂C₁₂N₄O: C, 55.35; H, 3.48; N, 16.14. Found: C, 55.25; H, 3.46; N, 16.03.

### EXAMPLE 131

### N′-((4-bromophenyl)methylidene)-4-chloro-3-methyl-1H-indole-2-carbohydrazide

The desired product was prepared by substituting Example 124C and 4-bromobenzaldehyde for Example 101C and 4-chlorobenzaldehyde, respectively, in Example 101D. MS (CI) m/e 390, 392, 394 (M+H)⁺; ¹H NMR (300 MHz, DMSO-d₆) δ 2.72 (s, 3H), 7.07 (d, 1H), 7.18 (t, 1H), 7.39 (d, 1H), 7.68 (s, 4H), 8.33 (s, 1H), 11.73 (br s, 2H); Anal. calcd. for C₁₇H₁₃BrClN₃O: C, 52.27; H, 3.35; N, 10.76. Found: C, 52.30; H, 3.24; N, 10.71.

### EXAMPLE 132

### N′-((4-bromophenyl)methylidene)-7-chloro-3-methyl-1H-indole-2-carbohydrazide

The desired product was prepared by substituting Example 112B and 4-bromobenzaldehyde for Example 101C and 4-chlorobenzaldehyde, respectively, in Example 101D. MS (CI) m/e 390, 392, 394 (M+H)⁺; ¹H NMR (300 MHz, DMSO-d₆) δ 2.55 (s, 3H), 7.11 (t, 1H), 7.37 (d, 1H), 7.65 (d, 1H), 7.70 (s, 4H), 7.35 (s, 1H), 11.44 (s, 1H) 11.76 (s, 1H); Anal. calcd. for C₁₇H₁₃BrClN₃O: C, 52.27; H, 3.35; N, 10.76. Found: C, 52.18; H, 3.33; N, 10.75.

### EXAMPLE 133

### 7-chloro-N′-((4-chlorophenyl)methylidene)-3-phenyl-1H-indole-2-carbohydrazide

### EXAMPLE 133A

### Ethyl 7-chloro-3-phenyl-1H-indole-2-carboxylate

The desired product was prepared by substituting ethyl 2-oxo-3-phenylpropionate for 2-oxobutyric acid ethyl ester in Example 112A. MS (CI) m/e 300 (M+H)⁺, 317 (M+NH₄)⁺.

### EXAMPLE 133B

### 7-chloro-3-phenyl-1H-indole-2-carbohydrazide

The desired product was prepared by substituting Example 133A for Example 101B in Example 101C and by using 30 equivalents of hydrazine hydrate and heating the reaction mixture to reflux for 2 days. MS (CI) m/e 286 (M+H)⁺.

### EXAMPLE 133C

### 7-chloro-N′-((4-chlorophenyl)methylidene)-3-phenyl-1H-indole-2-carbohydrazide

The desired product was prepared by substituting Example 133B for Example 101C in Example 101D. MS (CI) m/e 408, 410 (M+H)⁺; ¹H NMR (300 MHz, DMSO-d₆) δ 7.14 (t, 1H), 7.30-7.62 (m, 10H), 7.75 (d, 1H), 8.22 (s, 1H), 11.82 (s, 1H), 12.10 (s, 1H). Anal. calcd. for C₂₂H₁₅Cl₂N₃O, 64.72; H, 3.70; N, 10.29. Found: C, 64.52; H, 3.56; N, 10.26.

### EXAMPLE 134

### 7-chloro-N′-((4-cyanophenyl)methylidene)-3-phenyl-1H-indole-2-carbohydrazide

The desired product was prepared by substituting Example 133B and 4-formylbenzonitrile for Example 101C and 4-chlorobenzaldehyde, respectively, in Example 101D. MS (CI) 399 (M+H)⁺; ¹H NMR (300 MHz, DMSO-d₆) δ 7.15 (t, 1H), 7.24-7.70 (m, 7H), 7.91 (s, 4H), 8.28 (s, 1H), 11.95-12.15 (br d, 2H); Anal. calcd. for C₂₃H₁₅ClN₄O: C, 69.26; H, 3.79; N, 14.05. Found: C, 69.17; H, 3.54; N, 14.14.

### EXAMPLE 135

### 3-(methylsulfonyl)-N′-(1-naphthylmethylidene)-1H-indole-2-carbohydrazide

The desired product was prepared by substituting Example 110A and 1-naphthaldehyde for Example 103B and 4-chlorobenzaldehyde, respectively, in Example 103C. mp>250° C.; MS (ESI) m/e 392 (M+H)⁺, 414 (M+Na)⁺, 390 (M−H)⁻; ¹H NMR (DMSO-d₆) δ 3.07 (s, 1.33H), 3.40 (s, 1.67H), 6.67-6.73 (m, 0.44H), 7.32-7.74 (m, 6.56H), 7.87-8.21 (m, 3.56H), 8.62 ( s, 0.44H), 8.93-8.95 (m, 1H), 12.35 (s, 0.56H), 12.40 (s, 0.44), 12.84 (s, 0.44H), 12.99 (s, 0.56H). Anal. calcd. for C₂₁H₁₇N₃O₃S: C, 64.44; H, 4.38; N, 10.73. Found: C64.30; H, 4.23; N, 10.82.

### EXAMPLE 136

### N,N-dimethyl-2-((2-(2-naphthylmethylene)hydrazino)carbonyl)-1H-indole-3-carboxamide

The desired product was prepared by substituting Example 106A and 1-naphthaldehyde for Example 103B and 4-chlorobenzaldehyde, respectively, in Example 103C. mp 256-260° C.; MS (ESI) m/e 385 (M+H)⁺, 407 (M+Na)⁺, 383 (M−H)⁻; ¹H NMR (DMSO-d₆) δ 3.06 (br s, 6H), 7.19-7.24 (t, 1H), 7.29-7.34 (t, 1H), 7.55-7.60 (m, 4H), 7.95-8.02 (m, 4H), 8.26 (s, 1H), 8.43 (s, 1H), 12.36 (s, 1H), 12.64 (s, 1H); Anal. calcd. for C₂₃H₂₀N₄O₂: C, 71.86; H, 5.24; N, 14.57. Found: C, 71.62; H, 5.16; N, 14.52.

### EXAMPLE 137

### 7-chloro-3-phenyl-N′-(pyridin-3-ylmethylidene)-1H-indole-2-carbohydrazide

The desired product was prepared by substituting Example 133B and nicotinaldehyde for Example 101C and 4-chlorobenzaldehyde, respectively, in Example 101D. MS (CI) m/e 375 (M+H)⁺; ¹H NMR (300 MHz, DMSO-d₆) δ 7.15 (t, 1H), 7.20-7.70 (m, 9H), 8.14 (br d, 1H), 8.28 (s, 1H), 8.62 (br s, 1H), 8.85 (br s, 1H), 11.93 (br s, 1H), 12.11 (br s, 1H); Anal. calcd. for C₂₁H₁₅ClN₄O: C, 67.29; H, 4.03; N, 14.95. Found: C, 67.06; H, 3.90; N, 14.81.

### EXAMPLE 138

### 7-chloro-N′-(1H-imidazol-2-ylmethylidene)-3-phenyl-1H-indole-2-carbohydrazide

The desired product was prepared as a mixture of isomers by substituting Example 133B and 1H-imidazole-2-carbaldehyde for Example 101C and 4-chlorobenzaldehyde, respectively, in Example 101D. MS (CI) m/e 364 (M+H)⁺; ¹H NMR (300 MHz, DMSO-d₆) δ 6.89 (s, 0.7H), 7.10-7.65 (m, 10H), 8.10 (s, 0.3H). 11.76 (s, 0.3H), 12.17 (s, 1H), 12.76 (br s, 0.7H), 12.84 (br s, 0.3H), 13.87 (0.7H); Anal. calcd. for C₁₉H₁₄ClN₅O: C, 62.73; H, 3.88; N, 19.25. Found: C, 62.48; H, 3.74; N, 19.11.

### EXAMPLE 139

### N′-((4-chlorophenyl)methylidene)-7-fluoro-3-phenyl-1H-indole-2-carbohydrazide

### EXAMPLE 139A

### Ethyl 7-fluoro-3-phenyl-1H-indole-2-carboxylate

The desired product was prepared by substituting 2-florophenylhydrazine hydrochloride and ethyl 2-oxo-3-phenylpropionate for 2-chlorophenylhydrazine hydrochloride and 2-oxobutyric acid ethyl ester, respectively, in Example 112A. MS (CI) m/e 284 (M+H)⁺, 301 (M+NH₄)⁺.

### EXAMPLE 139B

### 7-fluoro-3-phenyl-1H-indole-2-carbohydrazide

The desired product was prepared by substituting Example 139A for Example 101B in Example 101C and by using 50 equivalents of hydrazine hydrate and heating the mixture to reflux for 2 days. MS (ESI) m/e 270 (M+H)⁺.

### EXAMPLE 139C

### N′-((4-chlorophenyl)methylidene)-7-fluoro-3-phenyl-1H-indole-2-carbohydrazide

The desired product was prepared by substituting Example 139B for Example 101C in Example 101D. MS (CI) m/e 392, 394 (M+H)⁺; ¹H NMR (300 MHz, DMSO-d₆) δ 7.11 (m, 2H), 7.35 (m, 2H), 7.40-7.60 (m, 6H), 7.74 (br d, 2H), 8.16 (s, 1H), 11.70 (s, 1H), 12.33 (s, 1H); Anal. calcd. for C₂₂H₁₅ClN₃O: C, 67.44; H, 3.86; N, 10.72. Found: C, 67.15; H, 3.76; N, 10.71.

### EXAMPLE 140

### N′-((4-cyanophenyl)methylidene)-7-fluoro-3-phenyl-1H-indole-2-carbohydrazide

The desired product was prepared by substituting Example 139B and 4-formylbenzonitrile for Example 101C and 4-chlorobenzaldehyde, respectively, in Example 101D. MS (CI) 383 (M+H)⁺; ¹H NMR (300 MHz, DMSO-d₆) δ 7.11 (m, 2H), 7.20-7.60 (br m, 7H), 7.90 (br s, 3H), 8.23 (br s, 1H), 11.88 (br s, 1H), 12.35 (br s, 1H); Anal. calcd. for C₂₃H₁₅FN₄O: C, 72.24; H, 3.95; N, 14.65. Found: C, 71.92; H, 3.93; N, 14.96.

### EXAMPLE 141

### 7-fluoro-3-phenyl-N′-(pyridin-3-ylmethylidene)-1H-indole-2-carbohydrazide

The desired product was prepared by substituting Example 139B and nicotinaldehyde for Example 101C and 4-chlorobenzaldehyde, respectively, in Example 101D. MS (CI) m/e 359 (M+H)⁺; ¹H NMR (300 MHz, DMSO-d₆) δ 7.11(m, 2H), 7.25-7.58 (br m, 8H), 8.13 (br d, 1H), 8.23 (s, 1H), 8.61 (br s, 1H), 8.84 (br s, 1H), 11.81 (br s, 1H), 12.34 (br s, 1H); HRMS (ESI) calcd. for C₂₁H₁₆N₄OF: 359.1308. Found: 359.1325.

### EXAMPLE 142

### 7-fluoro-N′-(1H-imidazol-2-ylmethylidene)-3-phenyl-1H-indole-2-carbohydrazide

The desired product was prepared by substituting Example 139B and 1H-imidazole-2-carbaldehyde for Example 101C and 4-chlorobenzaldehyde, respectively, in Example 101D. MS (CI) 348 (M+H)⁺; ¹H NMR (300 MHz, DMSO-d₆) δ 6.85 (br s, 1H), 7.40 (m, 3H), 7.23 (m, 1H), 7.32-7.45 (m, 4H), 7.45-7.54 (m, 2H), 12.47 (br s, 1H), 12.70 (br s, 1H), 13.85 (s, 1H); Anal. calcd. for C₁₉H₁₄FN₅O: C, 65.70; H, 4.06; N, 20.16. Found: C, 65.45; H, 3.89; N, 19.88.

### EXAMPLE 143

### N′-((4-bromophenyl)methylidene)-7-chloro-3-phenyl-1H-indole-2-carbohydrazide

The desired product was prepared by substituting Example 133B and 4-bromobenzaldehyde for Example 101C and 4-bromobenzaldehyde, respectively, in Example 101D. MS (CI) m/e 452, 454 (M+H)⁺; ¹H NMR (300 MHz, DMSO-d₆) δ 7.14 (t, 1H), 7.45-7.54 (m, 11H), 8.20 (s, 1H), 11.83 (s, 1H), 12.10 (s, 1H); Anal. calcd. for C₂₂H₁₅BrClN₃O: C, 58.36; H, 3.34; N, 9.28. Found: C, 58.16; H, 3.23; N, 9.23.

### EXAMPLE 144

### 7-chloro-N′-(1H-imidazol-5-ylmethylidene)-3-phenyl-1H-indole-2-carbohydrazide

The desired product was prepared by substituting Example 133B and 1H-imidazole-2-carbaldehyde for Example 101C and 4-chlorobenzaldehyde, respectively, in Example 101D. MS (CI) 364, 366 (M+H)⁺; ¹H NMR (300 MHz, DMSO-d₆) δ 7.13 (t, 1H), 7.21(t, 1H), 7.34-7.43 (m, 4H), 7.46-7.58 (m, 5H), 12.14 (br s, 1H), 12.60 (br s, 1H), 13.64 (s, 1H); Anal. calcd. for C₁₉H₁₄ClN₅O: C, 62.73; H, 3.88; N, 19.25. Found: C, 62.45; H, 3.77; N, 9.24.

### EXAMPLE 145

### 7-chloro-N′-(2-furylmethylidene)-3-phenyl-1H-indole-2-carbohydrazide

The desired product was prepared by substituting Example 133B and 2-furaldehyde for Example 101C and 4-chlorobenzaldehyde, respectively, in Example 101D. MS (CI) m/e 364, 366 (M+H)⁺; ¹H NMR (300 MHz, DMSO-d₆) δ 6.64 (br s, 1H), 6.94 (br s, 1H), 7.14 (t, 1H), 7.30-7.55 (m, 7H), 7.60 (d, 1H). 7.86 (br s, 1H), 8.08 (s, 1H), 11.67 (s, 1H), 12.11 (s, 1H); Anal. calcd. for C₂₀H₁₄ClN₃O₂: C, 66.03; H, 3.88; N, 11.55. Found: C, 65.84; H, 3.83; N, 11.54.

### EXAMPLE 146

### 7-chloro-3-phenyl-N′-(1,3-thiazol-2-ylmethylidene)-1H-indole-2-carbohydrazide

The desired product was prepared by substituting Example 133B and 1,3-thiazole-2-carbaldehyde for Example 101C and 4-chlorobenzaldehyde, respectively, in Example 101D. MS (CI) m/e 381, 383 (M+H)⁺, 398, 400 (M+NH₄)⁺; ¹H NMR (300 MHz, DMSO-d₆) δ 7.16 (t, 1H), 7.30-7.57 (m, 6H), 7.61 (d, 1H), 7.87 (br s, 1H), 7.97 (br s, 1H), 8.43 (br s, 1H), 12.10 (br s, 2H); Anal. calcd. for C₁₉H₁₃ClN₄OS: C, 59.92; H, 3.44; N, 14.71. Found: C, 59.85; H, 3.24; N, 14.63.

### EXAMPLE 147

### N′-((4-bromophenyl)methylidene)-7-fluoro-3-phenyl-1H-indole-2-carbohydrazide

The desired product was prepared by substituting Example 139B and 4-bromobenzaldehyde for Example 101C and 4-chlorobenzaldehyde, respectively, in Example 101D. MS (ESI) 434, 436 (M−H)⁻; ¹H NMR (300 MHz, DMSO-d₆) δ 5.11 (m, 3H), 7.20-7.60 (br m, 6H), 7.66 (br s, 3H), 8.15 (s, 1H), 11.70 (br s, 1H), 12.33 (br s, 1H).

### EXAMPLE 148

### 7-fluoro-N′-(1H-imidazol-4-ylmethylidene)-3-phenyl-1H-indole-2-carbohydrazide

The desired product was prepared by substituting Example 139B and 1H-imidazole-4-carbaldehyde for Example 101C and 4-chlorobenzaldehyde, respectively, in Example 101D. MS (CI) m/e 348 (M+H)⁺; ¹H NMR (300 MHz, DMSO-d₆) δ 7.04-7.16 (m, 2H), 7.21 (t, 1H), 7.32-7.43 (m, 5H), 7.48-7.53 (m, 4H), 12.42 (br s, 1H), 12.58 (br s, 1H), 13.62 (s, 1H); Anal. calcd. for C₁₉H₁₄FN₅O: C, 65.70; H, 4.06; N, 20.16. Found: C, 65.49; H, 3.94; N, 20.32.

### EXAMPLE 149

### N′-(2-furylmethylidene)-N-methyl-3-phenyl-1H-indole-2-carbohydrazide

### EXAMPLE 149A

### Tert-butyl 1-methylhydrazinecarboxylate

A solution of methylhydrazine (3 g, 65.2 mmol) in tert-butanol (40 mL) was treated with t-butyl-2,4,5-trichlorophenyl carbonate (18.5, 65.2 mmol) and triethylamine (9.1 mL), heated to 40 to 50° C. for 18 hours, and concentrated. The concentrate was purified by flash column chromatography on silica gel with 30% ethyl acetate/hexanes to provide the desired product (4.6 g, 44%).

### EXAMPLE 149B

### 2-benzyl 1-tert-butyl 1-methylhydrazine-1,2-dicarboxylate

A solution of Example 149A (4.2 g, 28.7 mmol) in chloroform (25 mL) and 1.2N NaOH (25 mL) at 0° C. was treated with benzylchloroformate (4.3 mL, 30.2 mmol), warmed to room temperature, and stirred for 18 hours. The organic phase was washed with water, dried (Na₂SO₄), filtered, and concentrated to provide the desired product (9.23 g, 100%). MS (ESI) m/e 281 (M+H)⁺, 298 (M+NH₄)⁺, 279 (M−H)⁻.

### EXAMPLE 149C

### Benzyl 2-methylhydrazinecarboxylate

A solution of Example 149B (28.7 mmol) in dichloromethane (15 mL) at 0° C. was treated with trifluoroacetic acid (15 mL), warmed to room temperature, stirred for one hour, and concentrated. The concentrate was diluted with saturated sodium bicarbonate and extracted three times with ethyl acetate. The combined extracts were washed with brine, dried (Na₂SO₄), filtered, and concentrated to provide the desired product (4.4 g, 85%). MS (ESI) m/e 181 (M+H)⁺.

### EXAMPLE 149D

### Benzyl 2-methyl-2-((3-phenyl-1H-indol-2-yl)carbonyl)hydrazinecarboxylate

A solution of 3-phenyl-1H-indole-2-carboxylic acid (500 mg, 2.1 mmol) in DMF (20 mL) at 0° C. was treated sequentially with HOBT (338 mg, 2.5 mmol), 4-methylmorpholine (274 μL, 2.5 mmol), Example 149C (450 mg, 2.5 mmol), and EDC (479 mg, 2.5 mmol), warmed to room temperature, stirred for 18 hours, diluted with water, and extracted three times with ethyl acetate. The combined extracts were washed twice with water and then brine, dried (Na₂SO₄), filtered, and concentrated. The concentrate was purified by flash column chromatography on silica gel with 30% ethyl acetate/dichloromethane to provide the desired product (695 mg, 83%). MS (ESI) m/e 400 (M+H)⁺, 398 (M−H)⁻.

### EXAMPLE 149E

### N-methyl-3-phenyl-1H-indole-2-carbohydrazide

A solution of Example 149D (100 mg, 0.25 mmol) in methanol (10 mL) and THF (6 mL) at room temperature was treated with 10% palladium on carbon (10 mg), stirred under hydrogen for 1.5 hours, filtered through diatomaceous earth (Celite®), and concentrated to provide the desired product (63.6 mg, 95.7%). MS (ESI) m/e 266 (M+H)⁺, 288 (M+Na)⁺, 264 (M−H)⁻.

### EXAMPLE 149F

### N′-(2-furylmethylidene)-N-methyl-3-phenyl-1H-indole-2-carbohydrazide

The desired product was prepared by substituting Example 149E and 2-furaldehyde for Example 103B and 4-chlorobenzaldehyde, respectively, in Example 103C. mp 196-199° C.; MS (ESI) m/e 344 (M+H)⁺, 366 (M+Na)⁺, 342 (M−H)⁻; ¹H NMR (DMSO-d₆) δ 3.36 (s, 3H), 6.48-6.49 (m, 1H), 6.54-6.55 (d, 1H), 7.08-7.17 (m, 2H), 7.23-7.34 (m, 5H), 7.48-7.50 (d, 1H), 7.63-7.66 (m, 3H), 11.70 (s, 1H); Anal. calcd. for C₂₁H₁₇N₃O2 0.25H₂O: C, 72.50; H, 5.07; N, 12.07. Found: C, 72.81; H, 5.06; N, 12.12.

### EXAMPLE 150

### N′-((4-cyanophenyl)methylidene)-N-methyl-3-phenyl-1H-indole-2-carbohydrazide

The desired product was prepared by substituting Example 149E and 4-formylbenzonitrile for Example 103B and 4-chlorobenzaldehyde, respectively, in Example 103C. mp 194-196° C.; MS (ESI) m/e 379 (M+H)⁺, 401 (M+Na)⁺, 377 (M−H)⁻; ¹H NMR (DMSO-d₆) δ 3.43 (s, 3H), 7.06-7.35 (m, 7H), 7.43-7.51(m, 3H), 7.68-7.80 (4H), 11.84 (s, 1H);

Anal. calcd. for C₂₄H₁₈N₄O: C, 76.17; H, 4.79; N, 14.80. Found: C, 76.06; H, 4.96; N, 14.74.

### EXAMPLE 151

### N′-((4-bromophenyl)methylidene)-N-methyl-3-phenyl-1H-indole-2-carbohydrazide

The desired product was prepared by substituting Example 149E and 4-bromobenzaldehyde for Example 103B and 4-chlorobenzaldehyde, respectively, in Example 103C. mp 210-212° C.; MS (ESI) m/e 432, 434 (M+H)⁺, 430, 432 (M−H)⁻; ¹H NMR (DMSO-d₆) δ 3.41 (s, 3H), 7.08-7.15 (m, 2H), 7.21-7.28(m, 5H), 7.34-7.37 (m, 2H), 7.47-7.50 (m, 3H), 7.68-7.75 (m, 2H), 11.79 (s, 1H); Anal. calcd. for C₂₃H₁₈BrN₃O: C, 63.90; H, 4.20; N, 9.72. Found: C,64.02; H, 4.29; N, 9.49.

### EXAMPLE 152

### N′-(1H-imidazol-5-ylmethylidene)-N-methyl-3-phenyl-1H-indole-2-carbohydrazide

The desired product was prepared by substituting Example 149E and 1H-imidazole-4-carbaldehyde for Example 103B and 4-chlorobenzaldehyde, respectively, in Example 103C.

mp 248-250° C.; MS (ESI) m/e 344 (M+H)⁺, 366 (M+Na)⁺, 342 (M−H)⁻; ¹H NMR (DMSO-d₆) δ 3.37 (s, 3H), 7.07-7.77 (m, 12H), 11.84 (s, 1H), 12.18 (s, 1H); Anal. calcd. for C₂₀H₁₇N₅O: C, 69.96; H, 4.99; N, 20.39. Found: C,69.89; H, 5.06; N, 20.30.

### EXAMPLE 153

### 3-phenyl-N′-(quinolin-3-ylmethylidene)-1H-indole-2-carbohydrazide

The desired product was prepared by substituting isoquinoline-3-carboxaldehyde for 4-bromobenzaldehyde in Example 2. MS (ESI(+)) m/e 391 (M+H)⁺, 781 (2M+H)⁺; ¹H NMR (300 MHz, DMSO-d₆) δ 7.14 (t, 1H), 7.31 (t, 1H), 7.39-7.60 (m, 3H), 7.61-7.72 (br m, 1H), 7.75-7.86 (br t, 1H), 7.98-8.09 (br d, 1H), 8.29 (br s, 0.5H), 8.58 (br s, 0.5H), (br s, 1H), 11.61 (br s, 0.5H), 11.94 (br s, 0.5H).

### EXAMPLE 154

### N′-((4-chloro-1-methyl-1H-pyrazol-3-yl)methylidene)-3-phenyl-1H-indole-2-carbohydrazide

The desired product was prepared by substituting 4-chloro-1-methylpyrazole-3-carboxaldehyde (60 mg, 0.42 mmol) for 4-bromobenzaldehyde in Example 2. MS (ESI(+)) m/e 378 (M+H)⁺, 400 (M+Na)⁺, 755 (2M+H)⁺, 777 (2M+Na)⁺; ¹H NMR (300 MHz, DMSO-d₆) δ 3.63 (s, 3H), 3.85 (s, 1H), 7.09-7.57 (m, 11H), 8.02 (s, 1H), 11.94 (s, 0.5H), 12.06 (s, 0.5H).

### EXAMPLE 155

### 3-isopropyl-N′-(1-naphthylmethylidene)-1H-indole-2-carbohydrazide

The desired product was prepared by substituting Example 20C and 1-naphthyladehyde for Example 1C and 4-bromobenzaldehyde, respectively, in Example 2. MS (ESI(−)) m/e 354 (M−H)⁻, 396 (M+Cl)⁻, 709 (2M−H)⁻; ¹H NMR (300 MHz, DMSO-d₆) δ 1.43 (d, 6H), 3.88 (br s, 1H), 7.05 (dt, 1H), 7.23 (dt, 1H), 7.45 (d, 1H), 7.56-7.71 (m, 2H), 7.82 (d, 1H), 7.92 (br d, 1H), 8.01-8.06 (m, 2H), 8.84-9.02 (br m, 1H), 11.34 (s, 1H), 11.71 (s, 1H).

### EXAMPLE 156

### 3-isopropyl-N′-(quinolin-4-ylmethylidene)-1H-indole-2-carbohydrazide

The desired product was prepared by substituting Example 20C and 4-quinoline carboxaldehyde for Example 1C and 4-bromobenzaldehyde, respectively, in Example 2. MS (ESI(+)) m/e 357 (M+H)⁺, 735 (2M+Na)⁺; ¹H NMR (300 MHz, DMSO-d₆) δ 1.43 (d, 6H), 3.87 (br s, 1H), 7.06 (d, 1H), 7.25 (d, 1H), 7.46 (d, 1H), 7.64-7.88 (m, 3H), 7.92 (br d, 1H), 8.11 (d, 1H), 8.72-9.02 (br m., 2H), 11.38 (s, 1H).

### EXAMPLE 157

### 3-isopropyl-N′-(isoquinolin-3-ylmethylidene)-1H-indole-2-carbohydrazide

The desired product was prepared by substituting Example 20C and 3-quinoline carboxaldehyde for Example 1C and 4-bromobenzaldehyde, respectively, in Example 2. MS (ESI(+)) m/e 357 (M+H)⁺, 713 (2M+H)⁺; ¹H NMR (300 MHz, DMSO-d₆) δ 1.43 (d, 6H), 3.87 (br s, 1H), 7.05 (d, 1H), 7.24 (d, 1H), 7.46 (d, 1H), 7.67-7.93 (m, 5H), 8.07-8.18 (m, 3H), 8.72-9.02 (br m., 2H), 11.39 (s, 1H), 11.95 (s, 1H).

### EXAMPLE 158

### 3-isopropyl-N′-(1,3-thiazol-2-ylmethylidene)-1H-indole-2-carbohydrazide

The desired product was prepared by substituting Example 20C and 1,3-thiazole-2-carbaldehyde for Example 1C and 4-bromobenzaldehyde, respectively, in Example 2. MS (ESI(+)) m/e 313 (M+H)⁺, 647 (2M+Na)⁺; ¹H NMR (300 MHz, DMSO-d₆) δ 1.42 (d, 6H), 3.80 (br s, 1H), 7.05 (d, 1H), 7.24 (d, 1H), 7.43 (d, 1H), 7.82 (d, 1H), 7.85 (d, 1H), 7.96 (d, 1H), 8.54 (s, 1H), 11.32 (s, 1H), 11.97 (s, 1H).

### EXAMPLE 159

### N′-(1H-imidazol-5-ylmethylidene)-3-isopropyl-1H-indole-2-carbohydrazide

The desired product was prepared by substituting Example 20C and 1H-imidazole-5-carbaldehyde for Example 1C and 4-bromobenzaldehyde, respectively, in Example 2. MS (ESI(+)) m/e 296 (M+H)⁺, 313 (M+NH₄)⁺, 613 (2M+Na)⁺; ¹H NMR (300 MHz, DMSO-d₆) δ 1.40 (d, 3H), 1.47 (d, 3H), 3.80 (br s, 1H), 7.03 (d, 1H), 7.19 (m, 1H), 7.34-7.63 (m, 2H), 7.72-7.85 (m, 2H), 8.03 (s, 0.5H), 8.26 (br s, 0.5H), 8.46-8.53 (m, 1H), 11.24-11.48 (m, 2H).

### EXAMPLE 160

### N′-(1H-imidazol-2-ylmethylidene)-3-isopropyl-1H-indole-2-carbohydrazide

The desired product was prepared by substituting Example 20C and 1H-imidazole-2-carbaldehyde for Example 1C and 4-bromobenzaldehyde, respectively, in Example 2. MS (ESI(+)) m/e 296 (M+H)⁺, 318 (M+Na)⁺, 591 (2M+H)⁺, 613 (2M+Na)⁺; ¹H NMR (300 MHz, DMSO-d₆) δ 1.40 (d, 6H), 4.02 (br s, 1H), 7.03-7.08 (m, 1H), 7.18-7.27 (m, 1H), 7.34-7.63 (m, 2H), 7.72-7.85 (m, 2H), 8.03 (s, 0.5H), 8.26 (br s, 0.5H), 8.46-8.53 (m, 1H), 11.24-11.68 (m, 2H).

### EXAMPLE 161

### N′-((3,4-dichlorophenyl)methylidene)-3-isopropyl-1H-indole-2-carbohydrazide

The desired product was prepared by substituting Example 20C and 3,4-dichlorobenzaldehyde for Example 1C and 4-bromobenzaldehyde, respectively, in Example 2. MS (ESI(+)) m/e 374 (M+H)⁺, 396 (M+Na)⁺, 749 (2M+H)⁺, 771 (2M+Na)⁺; ¹H NMR (300 MHz, DMSO-d₆) δ 1.41 (d, 6H), 3.83 (br s, 1H), 7.04 (t, 1H), 7.23 (t, 1H), 7.44 (d, 1H), 7.74 (s, 1H), 7.81 (d, 1H), 7.98 (s, 1H), 8.29 (d, 1H), 11.29 (s, 1H), 11.82 (s, 1H).

### EXAMPLE 162

### N′-((2,4-dichlorophenyl)methylidene)-3-isopropyl-1H-indole-2-carbohydrazide

The desired product was prepared by substituting Example 20C and 2,4-dichlorobenzaldehyde for Example 1C and 4-bromobenzaldehyde, respectively, in Example 2. MS (ESI(+)) m/e 374 (M+H)⁺, 396 (M+Na)⁺, 749 (2M+H)⁺, 771 (2M+Na)⁺; ¹H NMR (300 MHz, DMSO-d₆) δ 1.41 (d, 6H), 3.83 (br s, 1H), 7.04 (t, 1H), 7.23 (t, 1H), 7.43 (d, 1H), 7.54 (dd, 1H), 7.73 (d, 1H), 7.81 (d, 1H), 8.01 (d, 1H), 8.67 (d, 1H), 11.27 (s, 1H), 11.91 (s, 1H).

### EXAMPLE 163

### N′-((5-ethyl-2-furyl)methylidene)-3-isopropyl-1H-indole-2-carbohydrazide

The desired product was prepared by substituting Example 20C and 5-ethyl-2-furaldehyde for Example 1C and 4-bromobenzaldehyde, respectively, in Example 2. MS (ESI(+)) m/e 324 (M+H)⁺, 346 (M+Na)⁺, 647 (2M+H)⁺, 669 (2M+Na)⁺; ¹H NMR (300 MHz, DMSO-d₆) δ 1.21 (t, 3H), 1.40 (d, 6H), 2.70 (q, 2H), 3.77 (br s, 1H), 6.28 (d, 1H), 6.83 (d, 1H), 7.03 (t, 1H), 7.21 (t, 1H), 7.42 (d, 1H), 7.79 (d, 1H), 8.12 (br s, 1H), 11.25 (s, 1H), 11.49 (s, 1H).

### EXAMPLE 164

### N′-(1-benzofuran-2-ylmethylidene)-3-isopropyl-1H-indole-2-carbohydrazide

The desired product was prepared by substituting Example 20C and 1-benzofuran-2-carboxaldehyde for Example 1C and 4-bromobenzaldehyde, respectively, in Example 2. MS (ESI(+)) m/e 346 (M+H)⁺, 363 (M+NH₄)⁺, 691 (2M+H)⁺, 713 (2M+Na)⁺; ¹H NMR (300 MHz, DMSO-d₆) δ 1.41 (d, 6H), 3.72 (br s, 1H), 7.04 (t, 1H), 7.13 (t, 1H), 7.27-7.47 (m, 4H), 7.62-7.77 (m, 2H), 7.83 (d, 1H), 8.35 (s., 1H), 11.32 (s, 1H), 11.81 (s, 1H).

### EXAMPLE 165

### N′-((4-iodophenyl)methylidene)-3-phenyl-1H-indole-2-carbohydrazide

The desired product was prepared by substituting 4-iodobenzaldehyde for 4-bromobenzaldehyde in Example 2. MS (ESI(+)) m/e 466 (M+H)⁺, 488 (M+Na)⁺; ¹H NMR (300 MHz, DMSO-d₆) δ 7.13 (t, 1H), 7.29 (t, 1H), 7.33-7.57 (m, 7H), 7.65 (br d, 1H), 7.81 (br s, 2H), 8.02 (br s, 1H), 11.41 (br s, 1H), 11.91 (br s, 1H).

### EXAMPLE 166

### N′-((4-iodophenyl)methylidene)-3-isopropyl-1H-indole-2-carbohydrazide

The desired product was prepared by substituting Example 20C and 4-iodobenzaldehyde for Example 1C and 4-bromobenzaldehyde, respectively, in Example 2. MS (ESI(+)) m/e 432 (M+H)⁺, 454 (M+Na)⁺; ¹H NMR (300 MHz, DMSO-d₆) δ 1.40 (d, 6H), 3.82 (br s, 1H), 7.03 (t, 1H), 7.22 (t, 1H), 7.43 (d, 1H), 7.48-7.55 (m, 2H), 7.77-7.93 (m, 4H), 8.27 (s, 1H), 11.26 (s, 1H), 11.66 (s, 1H).

### EXAMPLE 167

### N′-(1-(4-cyanophenyl)ethylidene)-3-phenyl-1H-indole-2-carbohydrazide

The desired product was prepared by substituting 4-acetylbenzonitrile for 4-bromobenzaldehyde in Example 2. MS (ESI(+)) m/e 379 (M+H), 401 (M+Na)⁺; ¹H NMR (300 MHz, DMSO-d₆) δ 1.74 (br s, 3H), 7.11 (t, 1H), 7.29 (t, 1H), 7.34-7.58 (m, 6H), 7.82-7.96 (br s, 3H), 9.71 (br s, 1H), 12.03 (s, 1H).

### EXAMPLE 168

### N′-(1-(4-cyanophenyl)ethylidene)-3-isopropyl-1H-indole-2-carbohydrazide

The desired product was prepared by substituting Example 20C and 4-acetylbenzonitrile for Example 1C and 4-bromobenzaldehyde, respectively, in Example 2. MS (ESI(+)) m/e 345 (M+H)⁺; ¹H NMR (300 MHz, DMSO-d₆) δ 1.40 (d, 6H), 2.42 (s, 3H), 3.83 (br s, 1H), 7.03 (t, 1H), 7.23 (t, 1H), 7.44 (d, 1H), 7.81 (d, 1H), 7.90 (d., 2H), 8.00 (d, 2H), 10.66 (s. 1H), 11.42 (s, 1H).

### EXAMPLE 169

### N′-(1-(4-fluorophenyl)ethylidene)-3-isopropyl-1H-indole-2-carbohydrazide

The desired product was prepared by substituting Example 20C and 4-fluoroacetophenone for Example 1C and 4-bromobenzaldehyde, respectively, in Example 2. MS (ESI(+)) m/e 338 (M+H)⁺, 360 (M+Na)⁺, 697 (2M+Na)⁺; ¹H NMR (300 MHz, DMSO-d₆) δ 1.40 (d, 6H), 2.38 (s, 3H), 3.83 (br s, 1H), 7.02 (t, 1H), 7.17-7.31 (m, 3H), 7.43 (d, 1H), 7.80 (d, 1H), 7.84-7.92 (br m, 2H), 10.48 (s, 1H), 11.38 (s, 1H).

### EXAMPLE 170

### 3-isopropyl-N′-(1-(4-nitrophenyl)ethylidene)-1H-indole-2-carbohydrazide

The desired product was prepared by substituting Example 20C (109 mg, 0.5 mmol) and 4-nitroacetophenone (83 mg, 0.5 mmol) in 6 mL of ethanol for Example 1C and 4-bromobenzaldehyde in Example 2. 120 mg of the title compound was obtained. MS (ESI(+)) m/e 365 (M+H)⁺, 387 (M+Na)⁺, 751 (2M+Na)⁺; ¹H NMR (300 MHz, DMSO-d₆) δ 1.42 (d, 6H), 2.46 (s, 3H), 3.83 (br s, 1H), 7.03 (t, 1H), 7.23 (t, 1H), 7.44 (d, 1H), 7.82 (d, 1H), 8.08 (d, 2H), 8.28 (d, 2H), 10.72 (s, 1H), 11.44 (s, 1H).

### EXAMPLE 171

### N′-(1-(2-furyl)ethylidene)-3-isopropyl-1H-indole-2-carbohydrazide

The desired product was prepared by substituting Example 20C and 2-acetylfuran for Example 1C and 4-bromobenzaldehyde, respectively, in Example 2. MS (ESI(+)) m/e 310 (M+H)⁺, 332 (M+Na)⁺; ¹H NMR (300 MHz, DMSO-d₆) δ 1.41 (d, 6H), 2.32 (s, 3H), 3.84 (m, 1H), 6.62 (dd, 1H), 6.96 (d, 1H), 7.03 (dt, 1H), 7.22 (dt, 1H), 7.43 (d, 1H), 7.80 (dd, 1H), 10.43 (br s, 1H), 11.38 (s, 1H).

### EXAMPLE 172

### 3-isopropyl-N′-(1-(5-methyl-2-furyl)ethylidene)-1H-indole-2-carbohydrazide

The desired product was prepared by substituting Example 20C and 2-acetyl-4-methylfuran for Example 1C and 4-bromobenzaldehyde, respectively, in Example 2. MS (ESI(+)) m/e 324 (M+H)⁺, 346 (M+Na)⁺, 669 (2M+Na)⁺; ¹H NMR (300 MHz, DMSO-d₆) δ 1.41 (d, 6H), 2.27 (s, 3H), 2.45 (s, 3H), 3.86 (m, 1H), 6.23 (dd, 1H), 6.86 (d, 1H), 7.03 (dt, 1H), 7.22 (dt, 1H), 7.43 (d, 1H), 7.80 (d, 1H), 10.35 (br s, 1H), 11.39 (s, 1H).

### EXAMPLE 173

### N′-(1-(2-furyl)ethylidene)-3-phenyl-1H-indole-2-carbohydrazide

The desired product was prepared by substituting 2-acetylfuran for 4-bromobenzaldehyde in Example 2. MS (ESI(+)) m/e 344 (M+H)⁺, 366 (M+Na)⁺; ¹H NMR (300 MHz, DMSO-d₆) δ 2.53 (s, 3H), 6.57 (dd, 1H), 6.88 (br s, 1H), 7.09 (dt, 1H), 7.28 (dt, 1H), 7.40-7.59 (m, 7H), 7.77 (s, 1H), 9.40 (br s, 1H), 11.99 (s, 1H).

### EXAMPLE 174

### 3-isopropyl-N′(1-(1,3-thiazol-2-yl)ethylidene)-1H-indole-2-carbohydrazide

The desired product was prepared by substituting Example 20C and 2-acetylthiazole for Example 1C and 4-bromobenzaldehyde, respectively, in Example 2. Upon completion of the reaction the mixture was concentratedm treated with diethyl ether (4 mL), and cooled to 0° C. After 1 hour, the precipitate was collected by filtration, washed with cold diethyl ether and dried under vacuum to provide the desired product. MS (ESI(+)) m/e 327 (M+H)⁺, 349 (M+Na)⁺; ¹H NMR (300 MHz, DMSO-d₆) δ 1.42 (d, 6H), 2.52 (s, 3H), 3.85 (m, 1H), 7.03 (dt, 1H), 7.23 (dt, 1H), 7.44 (d, 1H), 7.77-7.84 (m, 2H), 10.77 (s, 1H), 11.42 (s, 1H).

### EXAMPLE 175

### N′-(1-(4-chlorophenyl)ethylidene)-3-isopropyl-1H-indole-2-carbohydrazide

The desired product was prepared by substituting Example 20C and 4′-chloroacetophenone for Example 1C and 4-bromobenzaldehyde, respectively, in Example 2. MS (ESI(+)) m/e 354 (M+H)⁺, 376 (M+Na)⁺, 729 (2M+Na)⁺; ¹H NMR (300 MHz, DMSO-d₆) δ 1.41 (d, 6H), 2.38 (s, 3H), 3.84 (m, 1H), 7.03 (dt, 1H), 7.22 (dt, 1H), 7.44 (d, 1H), 7.50 (d, 2H), 7.80 (d, 1H), 7.85 (d, 2H), 10.53 (s, 1H), 11.39(s, 1H).

### EXAMPLE 176

### 3-phenyl-N′-(1-pyridin-3-ylethylidene)-1H-indole-2-carbohydrazide

The desired product was prepared by substituting 3-acetylpyridine for 4-bromobenzaldehyde in Example 2. MS (ESI(+)) m/e 355 (M+H)⁺, 709 (2M+H)⁺, 731 (2M+Na)⁺; ¹H NMR (300 MHz, DMSO-d₆) δ 2.53 (s, 3H), 7.05-7.14 (m, 2H), 7.22-7.59 (m, 8H), 8.06 (br s, 1H), 8.56 (br d, 1H), 8.72-8.94 (m, 1H), 11.67 (s, 1H), 12.00 (s, 1H).

### EXAMPLE 177

### 3-isopropyl-N′-(1-pyridin-2-ylethylidene)-1H-indole-2-carbohydrazide

The desired product was prepared by substituting Example 20C and 2-acetylpyridine for Example 1C and 4-bromobenzaldehyde, respectively, in Example 2. MS (ESI(−)) m/e 319 (M−H)⁻, 639 (2M−H)⁻; ¹H NMR (300 MHz, DMSO-d₆) δ 1.42 (d, 6H), 2.48 (s, 3H), 3.85 (m, 1H), 7.03 (dt, 1H), 7.23 (dt, 1H), 7.38-7.47 (m, 2H), 7.82 (d, 1H), 7.87 (dd, 1H), 8.06 (br s, 1H), 8.63 (d, 1H), 10.61 (br s, 1H), 11.46 (s, 1H).

### EXAMPLE 178

### 3-isopropyl-N′-(1-pyridin-4-ylpropylidene)-1H-indole-2-carbohydrazide

The desired product was prepared by substituting Example 20C and 4-propionylpyridine for Example 1C and 4-bromobenzaldehyde, respectively, in Example 2. MS (ESI(−)) m/e 335 (M+H)⁺; ¹H NMR (300 MHz, DMSO-d₆) δ 1.09 (t, 3H), 1.40 (d, 6H), 2.94 (q, 2H), 3.75 (m, 1H), 7.03 (t, 1H), 7.22 (dt, 1H), 7.45 (d, 1H), 7.73 (d, 2H), 7.81 (d, 1H), 8.63 (d, 1H), 10.84 (br s, 1H), 11.38 (s, 1H).

### EXAMPLE 179

### 3-isopropyl-N′-(1H-pyrazol-3-ylmethylidene)-1H-indole-2-carbohydrazide

The desired product was prepared by substituting Example 20C and pyrrole-3-carboxaldehyde for Example 1C and 4-bromobenzaldehyde, respectively, in Example 2. MS (ESI(+)) m/e 296 (M+H)⁺, 318 (M+Na)⁺, 613 (2M+Na)⁺; ¹H NMR (300 MHz, DMSO-d₆) δ 1.47 (d, 6H), 2.94 (q, 2H), 3.75 (m, 1H), 6.72 (d, 1H), 7.03 (t, 1H), 7.21 (dt, 1H), 7.43 (d, 1H), 7.62 (s, 1H), 7.84 (d, 1H), 7.99 (d, 1H), 11.44 (s, 1H), 12.92 (s, 1H), 13.72 (s, 1H).

### EXAMPLE 180

### 3-phenyl-N′-(1H-pyrazol-3-ylmethylidene)-1H-indole-2-carbohydrazide

The desired product was prepared by substituting pyrrole-3-carboxaldehyde for 4-bromobenzaldehyde in Example 2. MS (ESI(+)) m/e 330 (M+H)⁺, 352 (M+Na)⁺, 681 (2M+Na)⁺; ¹H NMR (300 MHz, DMSO-d₆) δ 6.47 (s, 1H), 7.08-7.23 (m, 2H), 7.29 (t, 1H), 7.35-7.43 (m, 3H), 7.46-7.57 (m, 4H), 7.76 (s, 1H), 12.00 (s, 1H), 12.56 (s, 1H), 12.97 (s, 1H).

### EXAMPLE 181

### N′-((4-hydroxyphenyl)methylidene)-3-methyl-1H-indole-2-carbohydrazide

The desired product was prepared by substituting Example 61C and 4-hydroxybenzaldehyde for Example 1C and 4-bromobenzaldehyde, respectively, in Example 2.

MS (ESI(+)) m/e 294 (M+H)⁺; ¹H NMR (300 MHz, DMSO-d₆) δ 2.54 (s, 3H), 6.85 (d, 2H), 7.06 (t, 1H), 7.23 (t, 1H), 7.43 (d, 1H), 7.57 (d, 2H), 7.62 (d, 1H), 8.25 (br s, 1H), 9.92 (br s, 1H), 11.25 (br s, 2H).

### EXAMPLE 182

### N′-((4-iodophenyl)methylidene)-3-methyl-1H-indole-2-carbohydrazide

The desired product was prepared by substituting Example 61C and 4-iodobenzaldehyde for Example 1C and 4-bromobenzaldehyde, respectively, in Example 2. MS (ESI(+)) m/e 404 (M+H)⁺; ¹H NMR (300 MHz, DMSO-d₆) δ 2.50 (s, 3H), 7.07 (t, 1H), 7.24 (t, 1H), 7.42 (d, 1H), 7.53 (d, 2H), 7.64 (d, 1H), 7.84 (d, 2H), 8.3 (br s, 1H), 11.3 (br s, 1H), 11.52 (br s, 1H).

### EXAMPLE 183

### N′-((5-ethyl-2-furyl)methylidene)-3-phenyl-1H-indole-2-carbohydrazide

The desired product was prepared by substituting 5-ethyl-2-furaldehyde for 4-bromobenzaldehyde in Example 2. MS (ESI(+)) m/e 358 (M+H), 380 (M+Na)⁺, 715 (2M+Na)⁺, 737 (2M+Na)⁺; ¹H NMR (300 MHz, DMSO-d₆) δ 1.19 (t, 3H), 2.67 (q, 2H), 6.26 (s, 1H), 6.78 (s, 1H), 7.12 (t, 1H), 7.24-7.38 (m, 2H), 7.42-7.56 (m, 5H), 7.64 (d, 1H), 7.85 (s, 1H), 11.17 (s, 1H), 11.91 (s, 1H).

### EXAMPLE 184

### N′-((4-chlorophenyl)methylidene)-3-isopropoxy-1H-indole-2-carbohydrazide

### EXAMPLE 184A

### Methyl 2-((2-methoxy-2-oxoethyl)amino)benzoate

A solution of N-(2-carboxyphenyl)glycine (4.88 g, 25 mmol) and concentrated H₂SO₄ (7 mL) in methanol (60 mL) was heated to reflux for 18 hours and poured into ice/water (400 mL). The solid precipitate was filtered and partitioned between diethyl ether and saturated NaHCO₃. The organic phase was washed with brine, dried (Na₂SO₄), filtered, and concentrated. The concentrate was purified by flash column chromatography on silica gel with 10% ethyl acetate/hexanes to provide the desired product (64%). MS (ESI(+)) m/e 224 (M+H)⁺; ¹H NMR (300 MHz, DMSO-d₆) δ 3.68 (s, 3H), 3.82 (s, 3H), 4.14 (d, 2H), 6.65 (m, 2H), 7.38 (t, 1H), 7.82 (d, 1H), 7.98 (t, 1H).

### EXAMPLE 184B

### Methyl 3-hydroxy-1H-indole-2-carboxylate

A solution of potassium tert-butoxide (1.4 g, 12.5 mmol) in THF (25 mL) at 0° C. was treated slowly with a solution of Example 184A (2.0 g, 8.9 mmol) in THF (15 mL), heated to reflux for 2 hours, cooled to room temperature, poured into ice/water (200 mL), adjusted to pH <7 with glacial acetic acid, and extracted with ethyl acetate. The combined extracts were washed with brine, dried (Na₂SO₄), filtered, and concentrated. The concentrate was purified by flash column chromatography on silica gel with 10% ethyl acetate/hexanes to provide the desired product (75%). MS (ESI(−)) m/e 190 (M−H)⁺; ¹H NMR (300 MHz, DMSO-d₆) δ 3.82 (s, 3H), 6.95 (t, 1H), 7.21 (t, 1H), 7.28 (d, 1H), 7.70 (d, 1H), 9.28 (s, 1H), 10.83 (s, 1H).

### EXAMPLE 184C

### Methyl 3-isopropoxy-1H-indole-2-carboxylate

A solution of potassium tert-butoxide (0.25 g, 2.2 mmol) in DMSO (3 mL) at 0° C. was treated with a solution of Example 184B (0.3 g, 1.57 mmol) in DMSO (5 mL), warmed to room temperature, stirred for 1 hour, treated with 2-bromopropane (0.24 mL, 2.5 mmol), stirred at for 16 hours, and poured into ice/water (30 mL). The precipitate was filtered, washed with water and dried under vacuum at room temperature to provide the desired product (58%). MS (ESI(−)) m/e 232 (M−H)⁻; ¹H NMR (300 MHz, DMSO-d₆) δ 1.28 (d, 6H), 3.85 (s, 3H), 4.42-4.52 (m, 1H), 7.04 (t, 1H), 7.25 (t, 1H), 7.36 (d, 1H), 7.59 (d, 1H), 11.30 (s, 1H).

### EXAMPLE 184D

### 3-isopropoxy-1H-indole-2-carbohydrazide

The desired product was prepared by substituting Example 184C for Example 1B in Example 1C. MS (ESI(−)) m/e 232 (M−H)⁻; ¹H NMR (300 MHz, DMSO-d₆) δ 1.34 (d, 6H), 4.55 (s, 2H), 4.59-4.69 (m, 1H), 7.01 (t, 1H), 7.18 (t, 1H), 7.37 (d, 1H), 7.62 (d, 1H), 8.44 (s, 1H), 11.23 (s, 1H).

### EXAMPLE 184E

### N′-((4-chlorophenyl)methylidene)-3-isopropoxy-1H-indole-2-carbohydrazide

The desired product was prepared by substituting 184D and 4-chlorobenzaldehyde for Example 1C and 4-bromobenzaldehyde, respectively, in Example 2. MS (ESI(+)) m/e 356 (M+H)⁺; ¹H NMR (300 MHz, DMSO-d₆) δ 1.40 (d, 6H), 4.70-4.80 (m, 1H), 7.05 (t, 1H), 7.24 (t, 1H), 7.40 (d, 1H), 7.55 (d, 2H), 7.69 (d, 1H), 7.8 (d, 2H), 8.38 (s, 1H), 10.86 (s, 1H), 11.44 (s, 1H).

### EXAMPLE 185

### N′-((4-cyanophenyl)methylidene)-3-isopropoxy-1H-indole-2-carbohydrazide

The desired product was prepared by substituting Example 184D and 4-formylbenzonitrile for Example 1C and 4-bromobenzaldehyde, respectively, in Example 2.

MS (ESI(+)) m/e 347 (M+H)⁺; ¹H NMR (300 MHz, DMSO-d₆) δ 1.40 (d, 6H), 4.72-4.81 (m, 1H), 7.06 (t, 1H), 7.25 (t, 1H), 7.40 (d, 1H), 7.70 (d, 1H), 7.95 (s, 4H), 8.45 (s, 1H), 11.01 (s, 1H), 11.46 (s, 1H).

### EXAMPLE 186

### 3-isopropoxy-N′-((5-methyl-2-furyl)methylidene)-1H-indole-2-carbohydrazide

The desired product was prepared by substituting Example 184D and 5-methyl-2-furaldehyde for Example 1C and 4-bromobenzaldehyde, respectively, in Example 2. MS (ESI(+)) m/e 326 (M+H)⁺; ¹H NMR (300 MHz, DMSO-d₆) δ 1.38 (d, 6H), 2.37 (s, 3H), 4.66-4.76 (m, 1H), 6.28 (dd, 1H), 6.82 (d, 1H), 7.05 (t, 1H), 7.23 (t, 1H), 7.39 (d, 1H), 767 (d, 1H), 8.20 (s, 1H), 10.71 (s, 1H), 11.41 (s, 1H).

### EXAMPLE 187

### 3-isopropoxy-N′-(1,3-thiazol-2-ylmethylidene)-1H-indole-2-carbohydrazide

The desired product was prepared by substituting Example 184D and 1,3-thiazole-2-carbaldehyde for Example 1C and 4-bromobenzaldehyde, respectively, in Example 2.

MS (ESI(+)) m/e 329 (M+H)⁺; ¹H NMR (300 MHz, DMSO-d₆) δ 1.39 (d, 6H), 4.66-4.76 (m, 1H), 7.06 (t, 1H), 7.25 (t, 1H), 7.41 (d, 1H), 7.68 (d, 1H), 7.87 (d, 1H), 7.99 (d, 1H), 8.68 (s, 1H), 11.19 (s, 1H), 11.44 (s, 1H).

### EXAMPLE 188

### 3-chloro-N′-(phenylmethylidene)-1H-indole-2-carbohydrazide

### EXAMPLE 188A

### Ethyl 3-chloro-1H-indole-2-carboxylate

A mixture of ethyl 1H-indole-2-carboxylate (1.45 g, 7.67 mmol) and phosphorous pentachloride (1.6 g, 7.67 mmol) in toluene ( 10 mL) was heated to reflux for 30 minutes, cooled to room temperature, and filtered. The filter cake was washed with hexanes to provide the desired product (54%). MS (ESI(−)) m/e 222 (M−H)⁻; ¹H NMR (300 MHz, DMSO-d₆) δ 1.37 (t, 3H), 4.38 (q, 2H), 7.0 (t, 1H), 7.36 (t, 1H), 7.49 (d, 1H), 7.51 (d, 1H), 12.12 (s, 1H).

### EXAMPLE 188B

### 3-chloro-1H-indole-2-carbohydrazide

The desired product was prepared by substituting Example 188A for Example 1B in Example 1C. MS (ESI(−)) m/e 208 (M−H)⁻; ¹H NMR (300 MHz, DMSO-d₆) δ 4.54 (s, 2H), 7.17 (t, 1H), 7.29 (t, 1H), 7.45 (d, 1H), 7.55 (d, 1H), 9.18 (s, 1H), 11.86 (s, 1H).

### EXAMPLE 188C

### 3-chloro-N′-(phenylmethylidene)-1H-indole-2-carbohydrazide

The desired product was prepared by substituting Example 188B and benzaldehyde for Example 1C and 4-bromobenzaldehyde, respectively, in Example 2. MS (ESI(+)) m/e 298 (M+H)⁺; ¹H NMR (300 MHz, DMSO-d₆) δ 7.21 (t, 1H), 7.35 (t, 1H), 7.43-7.53 (m, 4H), 7.63 (d, 1H), 7.77 (br s, 2H), 8.44 (br s, 1H), 11.54 (br s, 1H).

### EXAMPLE 189

### 3-chloro-N′-((4-chlorophenyl)methylidene)-1H-indole-2-carbohydrazide

The desired product was prepared by substituting Example 188B and 4-chlorobenzaldehyde for Example 1C and 4-bromobenzaldehyde, respectively, in Example 2.

MS (ESI(+)) m/e 332 (M+H)⁺; ¹H NMR (300 MHz, DMSO-d₆) δ 7.21 (t, 1H), 7.35 (t, 1H), 7.50 (d, 1H), 7.55 (d, 2H), 7.62 (d, 1H), 7.74-7.83 (br d, 2H), 8.43 (br s, 1H), 11.60 (br s, 1H).

### EXAMPLE 190

### 3-chloro-N′-((4-cyanophenyl)methylidene)-1H-indole-2-carbohydrazide

The desired product was prepared by substituting Example 188B and 4-formylbenzonitrile for Example 1C and 4-bromobenzaldehyde, respectively, in Example 2. MS (ESI(+)) m/e 323 (M+H)⁺; ¹H NMR (300 MHz, DMSO-d₆) δ 7.21 (t, 1H), 7.35 (t, 1H), 7.50 (d, 1H), 7.63 (d, 1H), 7.93 (s, 4H), 8.48 (br s, 1H), 11.79 (br s, 1H).

### EXAMPLE 191

### 3-chloro-N′-((4-(difluoromethoxy)phenyl)methylidene)-1H-indole-2-carbohydrazide

The desired product was prepared by substituting Example 188B and 4-difluoromothoxybenzaldehyde for Example 1C and 4-bromobenzaldehyde, respectively, in Example 2. MS (ESI(+)) m/e 364 (M+H)⁺; ¹H NMR (300 MHz, DMSO-d₆) δ 7.08-7.58 (t, 1H), 7.20 (t, 1H), 7.28 (d, 2H), 7.44 (t, 1H), 7.50 (d, 1H),7.61 (d, 1H), 7.67-7.77 (br d, 2H), 8.42 (br s, 1H), 11.51 (br s, 1H).

### EXAMPLE 192

### 3-methyl-N′-(1,3-thiazol-2-ylmethylidene)-1H-indole-2-carbohydrazide

The desired product was prepared by substituting Example 61C and 1,3-thiazole-2-carbaldehyde for Example 1C and 4-bromobenzaldehyde, respectively, in Example 2. MS (ESI(+)) m/e 285 (M+H)⁺; ¹H NMR (300 MHz, DMSO-d₆) δ 2.54 (s, 3H), 7.09 (t, 1H), 7.25 (t, 1H), 7.44 (d, 1H), 7.65 (d, 1H), 7.86 (d, 1H), 7.98 (d, 1H), 8.58 (s, 1H), 11.33 (s, 1H), 11.82 (s, 1H).

### EXAMPLE 193

### N′-(1,3-thiazol-2-ylmethylidene)-3-(4-(trifluoromethoxy)phenyl)-1H-indole-2-carbohydrazide

### EXAMPLE 193A

### Ethyl 3-iodo-1H-indole-2-carboxylate

A solution of potassium hydroxide (8.82 g, 0.16 mol) in N,N-dimethylformamide (100 mL) at room temperature was stirred for 15 minutes, treated with ethyl 1H-indole-2-carboxylate (8.50 g, 0.045 mol), stirred for 5 minutes, treated dropwise with a solution of iodine (11.44 g, 0.045 mol) in N,N-dimethylformamide (50 mL), stirred for 40 minutes, poured into a mixture of sodium bisulfite (10.0 g), ammonia (100 mL), and water (1500 mL), and filtered. The filter cake was dried to provide the desired product (14.0 g). MS (ESI(+)) m/e 316 (M+H)⁺; ¹H NMR (300 MHz, DMSO-d₆) δ 1.39 (t, 3H), 4.38 (q, 2H), 7.18 (t, 1H), 7.34 (t, 1H), 7.44 (dd, 2H), 12.24 (br s, 1H).

### EXAMPLE 193B

### Ethyl 3-(4-(trifluoromethoxy)phenyl)-1H-indole-2-carboxylate

A solution of Example 193A (2.0 g, 6.3 mmol) in dimethoxyethane (55 mL) was treated sequentially with 2N Na₂CO₃ (12.6 mL), 4-trifluromethoxyboronic acid (1.3 g, 6.3 mmol), and (1,1′-bis-(diphenylphosphino)ferrocenedichloropalladium (II)) (33 mg, 0.045 mmol), heated to 84° C. for 18 hours on an Argonaut Quest, cooled to room temperature, and filtered. The filtrate was treated with water and extracted with ethyl acetate. The combined extracts were dried (Na₂SO₄), filtered, and concentrated to provide a 70:30 mixture of the desired product and a by-product (2.0 g) which was used without further purification. MS (ESI(+)) m/e 351 (M+2H)⁺; ¹H NMR (300 MHz, DMSO-d₆) δ 1.18 (t, 3H), 4.23 (q, 2H), 7.11 (t, 1H), 7.33 (t, 1H), 7.41-7.64 (m, 6H), 12.00 (br s, 1H).

### EXAMPLE 193C

### 3-(4-(trifluoromethoxy)phenyl)-1H-indole-2-carbohydrazide

A solution of Example 193B (2.0 g, 5.7 mmol) in ethanol (15 mL) was treated with hydrazine hydrate (1.8 mL, 57 mmol), heated to reflux for 18 hours, cooled to room temperature, and filtered. The filter cake was washed with ethanol and dried under vacuum to provide a mixture of the desired product and indole-2-carbohydrazide (1.1 g). MS (ESI(+)) m/e 336 (M+H)⁺; ¹H NMR (300 MHz, DMSO-d₆) δ 4.03 (br s, 2H), 7.09 (t, 1H), 7.25 (t, 1H), 7.41-7.62 (m, 6H), 9.13 (br s, 1H), 11.73(br s, 1H).

### EXAMPLE 193D

### N′-(1,3-thiazol-2-ylmethylidene)-3-(4-(trifluoromethoxy)phenyl)-1H-indole-2-carbohydrazide

A solution of Example 193C (50 mg, 0.14 mmol) and 1,3-thiazole-2-carbaldehyde (25.5 mg, 0.21 mmol) in ethanol (2.5 mL) was heated to 60° C. for 18 hours, cooled to room temperature, and concentrated. The concentrate was dissolved in methylsulfoxide and purified by preparative HPLC with acetonitrile/water containing 0.1% TFA to provide a mixture of isomers of the desired product (37.6 mg, 58.7%). MS (ESI(+)) m/e 431 (M+H)⁺; ¹H NMR (300 MHz, DMSO-d₆) δ 7.16 (t, 1H), 7.32 (t, 1H), 7.39 (d, 1H), 7.42 (br s, 1H) 7.54 (d, 1H), 7.57 (d, 1H), 7.63 (br s, 1H), 7.65-7.68 (m, 2H), 7.8 (br s, 1H), 7.9 (d, 1H), 11.99 (m, 1H), 13.45 (br s, 1H).

### EXAMPLE 194

### N′-((4-chlorophenyl)methylidene)-3-(4-(trifluoromethoxy)phenyl)-1H-indole-2-carbohydrazide

A solution of Example 193C (50 mg, 0.14 mmol) and 4-chlorobenzaldehyde (31.6 mg, 0.21 mmol) in ethanol (2.5 mL) was heated to 60° C. for 18 hours, cooled to room temperature, and concentrated. The concentrate was dissolved in methylsulfoxide and purified by preparative HPLC with 0.1% trifluoroacetic acid in acetonitrile to provide the desired product (5.9 mg, 8.6%). MS (ESI(+)) m/e 458 (M+H)⁺; ¹H NMR (300 MHz, DMSO-d₆) δ 7.15 (t, 1H), 7.31 (t, 1H), 7.42 (br s, 1H), 7.53 (d, 2H) 7.65 (m, 6H), 8.14 (br s, 2H), 11.58 (br s, 1H), 11.96 (br s, 1H).

### EXAMPLE 195

### N′-((4-bromophenyl)methylidene)-3-(4-(trifluoromethoxy)phenyl)-1H-indole-2-carbohydrazide

A solution of Example 193C (50 mg, 0.14 mmol) and 4-bromobenzaldehyde (41.6 mg, 0.21 mmol) in ethanol (2.5 mL) was heated to 60° C. for 18 hours, cooled to room temperature, and concentrated. The concentrate was dissolved in methylsulfoxide and purified by preparative HPLC with 0.1% trifluoroacetic acid in acetonitrile to provide the desired product (49.7 mg, 66.4%). MS (ESI(+)) m/e 503 (M+H)⁺; ¹H NMR (300 MHz, DMSO-d₆) δ 7.15 (t, 1H), 7.31 (t, 1H), 7.42 (br s, 1H), 7.53 (d, 1H) 7.63-7.66 (m, 7H), 8.12 (br s, 2H), 11.58 (br s, 1H), 11.95 (br s, 1H).

### EXAMPLE 196

### 3-(3-chlorophenyl)-N′-(1H-imidazol-2-ylmethylidene)-1H-indole-2-carbohydrazide

### EXAMPLE 196A

### Ethyl 3-(3-chlorophenyl)-1H-indole-2-carboxylate

A solution of Example 193A (2.0 g, 6.3 mmol) in dimethoxyethane (55 mL) was treated with 2N Na₂CO₃ (12.6 mL), 3-chlorophenylboronic acid (0.99 g, 6.3 mmol), and (1,1′-bis-(diphenylphosphino)ferrocenedichloropalladium (II)) (33 mg, 0.045 mmol), heated to 84° C. for 18 hours on an Argonaut Quest, cooled to room temperature, and filtered. The filtrate was treated with water and extracted with ethyl acetate. The combined extracts were dried (NaSO₄), filtered, and concentrated to provide an 80:20 mixture of the desired product and and a by-product (1.9 g) which was used without further purification. MS (ESI(+)) m/e 300 (M+H)⁺; ¹H NMR (300 MHz, DMSO-d₆) δ 1.20 (t, 3H), 4.24 (q, 2H), 7.18 (t, 1H), 7.28 (t, 1H), 7.44-7.56 (m, 6H), 9.87 (br s, 1H).

### EXAMPLE 196B

### 3-(3-chloro)-phenyl-1H-indole-2-carbohydrazide

A solution of Example 196A (1.9 g, 6.3 mmol) in ethanol (15 mL) was treated with hydrazine hydrate (2.0 mL, 63 mmol), heated to reflux for 18 hours, cooled to room temperature, and filtered. The filter cake was washed with ethanol and dried under vacuum to provide the desired product (0.96 g, 53%). MS (ESI(+)) m/e 268 (M+H)⁺; ¹H NMR (300 MHz, DMSO-d₆) δ 4.43(br s, 2H), 7.14 (t, 1H), 7.23 (t, 1H), 7.41-7.62 (m, 6H), 9.17 (br s, 1H), 11.78 (br s, 1H).

### EXAMPLE 196C

### 3-(3-chlorophenyl)-N′-(1H-imidazol-2-ylmethylidene)-1H-indole-2-carbohydrazide

A solution of Example 196B (50 mg, 0.18 mmol) and 1H-imidazole-2-carbaldehyde (24.5 mg, 0.27 mmol) in ethanol (2.5 mL) was heated to 60° C. for 18 hours, cooled to room temperature, and concentrated. The concentrate was dissolved in methylsulfoxide and purified by preparative HPLC with 0.1% trifluoroacetic acid in acetonitrile to provide the desired product as a mixture of isomers (8.9 mg, 14%). MS (ESI(+)) m/e 364 (M+H)⁺; ¹H NMR (300 MHz, DMSO-d₆) δ 7.18 (t, 1H), 7.32-7.35 (m, 2H), 7.44 (br s, 1H), 7.54-7.58 (m, 6H), 7.63-7.66 (m, 2H), 8.10 (br s, 1H), 12.08 (br s, 1H).

### EXAMPLE 197

### 3-(3-chlorophenyl)-N′-(1H-imidazol-4-ylmethylidene)-1H-indole-2-carbohydrazide

A solution of Example 196B (50 mg, 0.18 mmol) and 1H-imidazole-4-carbaldehyde (24.5 mg, 0.27 mmol) in ethanol (2.5 mL) was heated to 60° C. for 18 hours, cooled to room temperature, and concentrated. The concentrate was dissolved in methylsulfoxide and purified by preparative HPLC with 0.1% trifluoroacetic acid in acetonitrile to provide the desired product as a mixture of isomers (9.8 mg, 15.4%). MS (ESI(+)) m/e 364 (M+H)⁺; ¹H NMR (300 MHz, DMSO-d₆) δ 7.11-7.18 (m, 1H), 7.27-7.33 (m, 2H), 7.39-7.46 (m, 4H), 7.50-7.54 (m, 3H), 7.64 (d, 1H), 8.14 (br s, 1H), 11.74 (br s, 1H), 12.02 (br s, 1H).

### EXAMPLE 198

### 3-(3-chlorophenyl)-N′-(1,3-thiazol-2-ylmethylidene)-1H-indole-2-carbohydrazide

A solution of Example 196B (50 mg, 0.18 mmol) and 1,3-thiazole-2-carbaldehyde (28.9 mg, 0.27 mmol) in ethanol (2.5 mL) was heated to 60° C. for 18 hours, cooled to room temperature, and concentrated. The concentrate was dissolved in methylsulfoxide and purified by preparative HPLC with 0.1% trifluoroacetic acid in acetonitrile to provide the desired product as a mixture of isomers (16.3 mg, 24.5%). MS (ESI(+)) m/e 381 (M+H)⁺; ¹H NMR (300 MHz, DMSO-d₆) δ 7.16 (d, 1H), 7.27 (d, 1H), 7.32 (t, 1H), 7.42-7.45 (m, 2H), 7.49-7.56 (m, 2H), 7.61-7.65 (m, 2H), 7.82 (s, 1H), 7.92 (br s, 1H), 12.14 (br s, 1H), 13.45 (br s, 1H).

### EXAMPLE 199

### 3-(3-chlorophenyl)-N′-(pyridin-3-ylmethylidene)-1H-indole-2-carbohydrazide

A solution of Example 196B (50 mg, 0.18 mmol) and nicotinaldehyde (27.3 mg, 0.27 mmol) in ethanol (2.5 mL) was heated to 60° C. for 18 hours, cooled to room temperature, and concentrated. The concentrate was dissolved in methylsulfoxide and purified by preparative HPLC with 0.1% trifluoroacetic acid in acetonitrile to provide the desired product. MS (ESI(+)) m/e 375 (M+H)⁺; ¹H NMR (300 MHz, DMSO-d₆) δ 7.16 (t, 1H), 7.32 (t, 1H), 7.45 (br s, 1H), 7.54 (m, 6H), 7.65 (d, 2H), 8.22 (br s, 1H), 8.63 (br s, 1H), 11.78 (br s, 1H), 12.00 (br s, 1H).

### EXAMPLE 200

### N′-((4-bromophenyl)methylidene)-3-(3-chlorophenyl)-1H-indole-2-carbohydrazide

A solution of Example 196B (50 mg, 0.18 mmol) and 4-bromobenzaldehyde (47.18 mg, 0.27 mmol) in ethanol (2.5 mL) was heated to 60° C. for 18 hours, cooled to room temperature and concentrated. The concentrate was dissolved in methylsulfoxide and purified by preparative HPLC with 0.1% trifluoroacetic acid in acetonitrile to provide the desired product (69.2 mg, 87.3%). MS (ESI(+)) m/e 454 (M+H)⁺; ¹H NMR (300 MHz, DMSO-d₆) δ 7.16 (t, 1H), 7.31 (t, 1H), 7.35-7.55 (m, 7H), 7.65 (m, 3H), 8.13 (br s, 1H), 11.58 (br s, 1H), 11.98 (br s, 1H).

### EXAMPLE 201

### N′-(1H-imidazol-2-ylmethylidene)-3-thien-2-yl-1H-indole-2-carbohydrazide

### EXAMPLE 201A

### Ethyl 3-thien-2-yl-1H-indole-2-carboxylate

A solution of Example 193A (2.0 g, 6.3 mmol) in dimethoxyethane (55 mL) was treated with 2N Na₂CO₃ (12.6 mL), 2-thiopheneboronic acid (0.81 g, 6.3 mmol), and (1,1′-bis-(diphenylphosphino)ferrocenedichloropalladium (II)) (33 mg, 0.045 mmol), heated to 84° C. for 18 hours on an Argonaut Quest, cooled to room temperature, and filtered. The filtrate was treated with water and extracted with ethyl acetate. The combined extracts were dried (Na₂SO₄), filtered, and concentrated to provide a 70:30 mixture of the desired product and a by-product (1.7 g) which was used without further purification. MS (ESI(+)) m/e 272 (M+H)⁺; ¹H NMR (300 MHz, DMSO-d₆) δ 1.27(t, 3H), 4.29 (q, 2H), 7.12-7.22 (m, 1H), 7.30-7.37 (m, 1H), 7.41-7.53 (m, 2H), 7.62-7.64 (m, 2H), 7.71 (d, 1H), 12.02 (br s, 1H).

### EXAMPLE 201B

### 3-thien-2-yl-1H-indole-2-carbohydrazide

A solution of Example 201A (1.7 g, 6.3 mmol) in ethanol (15 mL) was treated with hydrazine hydrate (2.0 mL, 63 mmol), heated to reflux for 18 hours, cooled to room temperature, and filtered. The filter cake was washed with ethanol and dried under vacuum to provide an 80:20 mixture of the desired product and the 3-unsubstituted by-product (0.91 g, 56%). MS (ESI(+)) m/e 258 (M+H)⁺; ¹H NMR (300 MHz, DMSO-d₆) δ 4.52 (br s, 2H), 7.00-7.31 (m, 2H), 7.40-7.71 (m, 5H), 9.03 (br s, 1H), 11.80 (br s, 1H).

### EXAMPLE 201C

### N′-(1H-imidazol-2-ylmethylidene)-3-thien-2-yl-1H-indole-2-carbohydrazide

A solution of Example 201B (50 mg, 0.19 mmol) and 1H-imidazole-2-carbaldehyde (27.4 mg, 0.29 mmol) in ethanol (2.5 mL) was heated to 60° C. for 18 hours, cooled to room temperature, and concentrated. The concentrate was dissolved in methylsulfoxide and purified by preparative HPLC with 0.1% trifluoroacetic acid in acetonitrile to provide the desired product as a mixture of stereoisomers (51 mg, 78%). MS (ESI(+)) m/e 336 (M+H)⁺; ¹H NMR (300 MHz, DMSO-d₆) δ 7.15 (br s, 2H), 7.19 (t, 1H), 7.33 (t, 1H), 7.51-7.56 (m, 6H), 7.83 (d, 1H), 8.16 (br s, 1H), 12.07 (br s, 1H).

### EXAMPLE 202

### N′-(1,3-thiazol-2-ylmethylidene)-3-thien-2-yl-1H-indole-2-carbohydrazide

A solution of Example 201B (50 mg, 0.19 mmol) and 1,3-thiazole-2-carbaldehyde (32.2 mg, 0.29 mmol) in ethanol (2.5 mL) was heated to 60° C. for 18 hours, cooled to room temperature and concentrated. The concentrate was dissolved in methylsulfoxide and purified by preparative HPLC with 0.1% trifluoroacetic acid in acetonitrile to provide the desired product as a mixture of isomers (16.1 mg, 24%). MS (ESI(+)) m/e 353 (M+H)⁺; ¹H NMR (300 MHz, DMSO-d₆) δ 7.09-7.20 (m., 3H), 7.32 (m, 2H), 7.51 (d, 1H), 7.57 (m, 2H), 7.83 (d, 1H), 8.39 (br s, 1H), 12.04 (m, 2H).

### EXAMPLE 203

### N′-(pyridin-3-ylmethylidene)-3-thien-2-yl-1H-indole-2-carbohydrazide

A solution of Example 201B (50 mg, 0.19 mmol) and nicotinaldehyde (30.5 mg, 0.29 mmol) in ethanol (2.5 mL) was heated to 60° C. for 18 hours, cooled to room temperature, and concentrated. The concentrate was dissolved in methylsulfoxide and purified by preparative HPLC with 0.1% trifluoroacetic acid in acetonitrile to provide the desired product (43.1 mg, 64.1%). MS (ESI(−)) m/e 345 (M−H)⁻; ¹H NMR (300 MHz, DMSO-d₆) δ 7.18 (m, 2H), 7.31 (m., 2H), 7.50-7.57 (m, 4H), 7.82 (d, 1H), 8.25 (br s, 1H), 8.65 (br s, 1H), 8.90 (br s, 1H), 11.84 (br s, 1H), 12.04 (br s, 1H).

### EXAMPLE 204

### N′-((4-chlorophenyl)methylidene)-3-thien-2-yl-1H-indole-2-carbohydrazide

A solution of Example 201B (50 mg, 0.19 mmol) and 4-chlorobenzaldehyde (40.6 mg, 0.29 mmol) in ethanol (2.5 mL) was heated to 60° C. for 18 hours, cooled to room temperature, and concentrated. The concentrate was dissolved in methylsulfoxide and purified by preparative HPLC with 0.1% trifluoroacetic acid in acetonitrile to provide the desired product (36.9 mg, 50.1%). MS (ESI(+)) m/e 380 (M+H)⁺; ¹H NMR (300 MHz, DMSO-d₆) δ 7.17 (t, 1H), 7.30 (t, 1H), 7.35 (br s, 1H), 7.46-7.57 (m, 6H), 7.75 (br s, 1H), 7.82 (d, 1H) 8.17 (br s, 1H), 11.61 (br s, 1H), 12.00 (br s, 1H).

### EXAMPLE 205

### N′-((4-bromophenyl)methylidene)-3-thien-2-yl-1H-indole-2-carbohydrazide

A solution of Example 201B (50 mg, 0.19 mmol) and 4-bromobenzaldehyde (52.7 mg, 0.29 mmol) in ethanol (2.5 mL) was heated to 60° C. for 18 hours, cooled to room temperature, and concentrated. The concentrate was dissolved in methylsulfoxide and purified by preparative HPLC with 0.1% trifluoroacetic acid in acetonitrile to provide the desired product (44.5 mg, 54.1%). MS (ESI(+)) m/e 425 (M+H)⁺; ¹H NMR (300 MHz, DMSO-d₆) δ 7.17 (t, 1H), 7.30 (t, 1H), 7.35 (br s, 1H), 7.50 (d, 1H), 7.57-7.68 (m, 6H), 7.81 (d, 1H) 8.15 (br s., 1H), 11.62 (br s, 1H), 12.01 (br s, 1H).

### EXAMPLE 206

### N′-((4-bromophenyl)methylidene)-3-(4-(dimethylamino)phenyl)-1H-indole-2-carbohydrazide

### EXAMPLE 206A

### Ethyl 3-(4-(dimethylamino)phenyl)-1H-indole-2-carboxylate

A solution of Example 193A (2.0 g, 6.3 mmol) in dimethoxyethane (55 mL) was treated with 2N Na₂CO₃ (12.6 mL), 4-N,N-dimethylaminophenylboronic acid (1.0 g, 6.3 mmol), and (1,1′-bis-(diphenylphosphino)ferrocenedichloropalladium (II)) (33 mg, 0.045 mmol), heated to 84° C. for 18 hours on an Argonaut Quest, cooled to room temperature, and filtered. The filtrate was treated with water and extracted with ethyl acetate. The combined extracts were dried (Na₂SO₄), filtered, and concentrated to provide a 70:30 mixture of the desired product and a by-product (1.7 g) which was used without further purification. MS (ESI(+)) m/e 309 (M+H)⁺; ¹H NMR (300 MHz, DMSO-d₆) δ 1.23 (t, 3H), 2.87 (s, 3H), 2.96 (s, 3H), 4.24 (q, 2H), 6.06-6.82 (m, 2H), 7.03-7.53 (m, 6H), 11.68 (br s, 1H).

### EXAMPLE 206B

### 3-(4-(dimethylamino)phenyl)-1H-indole-2-carbohydrazide

A solution of Example 206A (1.7 g, 5.7 mmol) in ethanol (15 mL) was treated with hydrazine hydrate (1.8 mL, 57 mmol), heated to reflux for 18 hours, cooled to room temperature, and filtered. The filter cake was washed with ethanol and dried under vacuum to provide a mixture of the desired product and the 3-unsubstituted product (0.48 g, 28%) which was used without further purification. MS (ESI(+)) m/e 295 (M+H)⁺; ¹H NMR (300 MHz, DMSO-d₆) δ 2.97 (s, 6H), 4.32(br s., 2H), 6.85 (d, 2H), 6.99-7.06 (m, 2H), 7.17 (t, 1H), 7.31 (d, 2H), 7.43 (d, 1H), 8.21 (br s, 1H), 11.54 (br s, 1H).

### EXAMPLE 206C

### N′-((4-bromophenyl)methylidene)-3-(4-(dimethylamino)phenyl)-1H-indole-2-carbohydrazide

A solution of Example 206B (50 mg, 0.17 mmol) and 4-bromobenzaldehyde (47.2 mg, 0.27 mmol) in ethanol (2.5 mL) was heated to 60° C. for 18 hours, cooled to room temperature, and concentrated. The concentrate was dissolved in methylsulfoxide and purified by preparative HPLC with 0.1% trifluoroacetic acid in acetonitrile to provide the desired product (42.1 mg, 53.7%). MS (ESI(+)) m/e 463 (M+H)⁺; ¹H NMR (300 MHz, DMSO-d₆) δ 2.97 (s, 6H), 6.94 (br s, 2H), 7.10 (t, 1H), 7.26 (t, 1H), 7.40 (m, 2H), 7.48 (d, 2H), 7.61 (s, 4H), 8.02 (br s, 1H), 11.13 (br s, 1H), 11.72 (br s, 1H).

### EXAMPLE 207

### 3-(2-chlorophenyl)-N′-(1H-imidazol-2-ylmethylidene)-1H-indole-2-carbohydrazide

### EXAMPLE 207A

### Ethyl 3-(2-chlorophenyl)-1H-indole-2-carboxylate

A solution of Example 193A (2.0 g, 6.3 mmol) in dimethoxyethane (55 mL) was treated with 2N Na₂CO₃ (12.6 mL), 2-chlorophenylboronic acid (1.0 g, 6.3 mmol), and (1,1′-bis-(diphenylphosphino)ferrocenedichloropalladium (II)) (33 mg, 0.045 mmol), heated to 84° C. for 18 hours on an Argonaut Quest, cooled to room temperature, and filtered. The mixture was treated with water and extracted with ethyl acetate. The combined extracts were dried (Na₂SO₄), filtered, and concentrated to provide a 70:30 mixture of product and by-product (1.7 g) which was used without further purification. MS (ESI(+)) m/e 300 (M+H)⁺; ¹H NMR (300 MHz, DMSO-d₆) δ 1.07(t, 3H), 4.34 (q, 2H), 7.08 (t, 1H), 7.12-7.66 (m, 7H), 10.09 (br s, 1H).

### EXAMPLE 207B

### 3-(2-chlorophenyl)-1H-indole-2-carbohydrazide

A solution of Example 207B (1.7 g, 5.67 mmol) in ethanol (15 mL) was treated with hydrazine hydrate (1.8 mL, 56 mmol), heated to reflux for 18 hours, cooled to room temperature, and filtered. The filter cake was washed with ethanol and dried under vacuum to provide a mixture of the desired product and the 3-unsubstituted product (0.64 g) which was used without further purification. MS (ESI(+)) m/e 286 (M+H)⁺; ¹H NMR (300 MHz, DMSO-d₆) δ 4.44 (br s, 2H), 7.02-7.07 (m, 1H), 7.17-7.27 (m, 2H), 7.40-7.49 (m, 4H), 7.56-7.59 (m, 1H), 8.56 (br s, 1H), 11.69(br s, 1H).

### EXAMPLE 207C

### 3-(2-chlorophenyl)-N′-(1H-imidazol-2-ylmethylidene)-1H-indole-2-carbohydrazide

A solution of Example 207B (50 mg, 0.18 mmol) and 1H-imidazole-2-carbaldehyde (24.5 mg, 0.27 mmol) in ethanol (2.5 mL) was heated to 60° C. for 18 hours, cooled to room temperature, and concentrated. The concentrate was dissolved in methylsulfoxide and purified by preparative HPLC with 0.1% trifluoroacetic acid in acetonitrile to provide the desired product (39.3 mg, 61.7%) as a mixture of two isomers. MS (ESI(−)) m/e 362 (M−H)⁻; ¹H NMR (300 MHz, DMSO-d₆) δ 7.12 (t, 1H), 7.24-7.34 (m, 3H), 7.37 (br s, 1H), 7.50-7.56 (m, 6H), 8.10(br s, 1H), 11.83 (br s, 1H), 11.98 (br s, 1H).

### EXAMPLE 208

### 3-(2-chlorophenyl)-N′-(1H-imidazol-4-ylmethylidene)-1H-indole-2-carbohydrazide

A solution of Example 207B (50 mg, 0.18 mmol) and 1H-imidazole-4-carbaldehyde (24.5 mg, 0.27 mmol) in ethanol (2.5 mL) was heated to 60° C. for 18 hours, cooled to room temperature, and concentrated. The concentrate was dissolved in methylsulfoxide and purified by preparative HPLC with 0.1% trifluoroacetic acid in acetonitrile to give 53.9 mg (84.7%) of a mixture of two isomers of desired product. MS (ESI(−)) m/e 363 (M−H)⁻; ¹H NMR (300 MHz, DMSO-d₆) δ 7.11 (t, 1H), 7.24-7.33 (m, 2H), 7.39-7.42 (m, 4H), 7.48-7.55 (m, 3H), 7.98 (br s, 1H), 9.03 (s, 1H), 11.55 (br s, 1H), 11.97 (br s, 1H).

### EXAMPLE 209

### 3-(2-chlorophenyl)-N′-(pyridin-3-ylmethylidene)-1H-indole-2-carbohydrazide

A solution of Example 207B (50 mg, 0.18 mmol) and nicotinaldeyhde (27.3 mg, 0.27 mmol) in ethanol (2.5 mL) was heated to 60° C. for 18 hours, cooled to room temperature, and concentrated. The concentrate was dissolved in methylsulfoxide and purified by preparative HPLC with 0.1% trifluoroacetic acid in acetonitrile to provide the desired product (58.7 mg, 89.5%). MS (ESI(−)) m/e 375 (M+H)⁺; ¹H NMR (300 MHz, DMSO-d₆) δ 7.10 (t, 1H), 7.29-7.33 (m, 3H), 7.37 (br s, 1H), 7.50 (m, 2H), 7.54-7.58 (m, 3H), 8.17 (br s, 1H), 8.64 (br s, 1H), 8.82 (br s, 1H), 11.55 (br s, 1H), 11.92 (br s, 1H).

### EXAMPLE 210

### 3-(2-chlorophenyl)-N′-((4-chlorophenyl)methylidene)-1H-indole-2-carbohydrazide

A solution of Example 207B (50 mg, 0.18 mmol) and 4-chlorobenzaldehyde (35.85 mg, 0.27 mmol) in ethanol (2.5 mL) was heated to 60° C. for 18 hours, cooled to room temperature, and concentrated. The concentrate was dissolved in 0.1% trifluoroacetic acid in acetonitrile to provide the desired product (52.5 mg, 73.5%). MS (ESI(+)) m/e 408 (M+H)⁺; ¹H NMR (300 MHz, DMSO-d₆) δ 7.10 (t, 1H), 7.28-7.32 (m, 3H), 7.40 (s, 1H), 7.49 (m, 4H), 7.54 (d, 2H), 7.68 (br s, 1H), 8.12 (br s, 1H), 11.30 (br s, 1H), 11.89 (br s, 1H).

### EXAMPLE 211

### N′-((4-bromophenyl)methylidene)-3-(2-chlorophenyl)-1H-indole-2-carbohydrazide

A solution of Example 207B (50 mg, 0.18 mmol) and 4-bromobenzaldehyde (47.18 mg, 0.27 mmol) in ethanol (2.5 mL) was heated to 60° C. for 18 hours, cooled to room temperature, and concentrated. The concentrate was dissolved in methylsulfoxide and purified by preparative HPLC with 0.1% trifluoroacetic acid in acetonitrile to provide the desired product (48.4 mg, 61.1%). MS (ESI(+)) m/e 453 (M+H)⁺; ¹H NMR (300 MHz, DMSO-d₆) δ 7.10 (t, 1H), 7.28-7.32 (m, 3H), 7.41 (s, 1H), 7.49 (m, 4H), 7.54 (d, 2H), 7.62 (br s, 1H), 8.10 (br s, 1H), 11.31 (br s, 1H), 11.89 (br s, 1H).

### EXAMPLE 212

### 3-pyridin-4-yl-N′-(1,3-thiazol-2-ylmethylidene)-1H-indole-2-carbohydrazide

### EXAMPLE 212A

### Ethyl 3-pyridin-4-yl-1H-indole-2-carboxylate

A solution of Example 193A (0.5 g, 1.6 mmol) in dimethoxyethane (20 mL) was treated with 2N Na₂CO₃ (3.2 mL), 4-pyridylboronic acid (0.20 g, 1.63 mmol), and (1,1′-bis-(diphenylphosphino)ferrocenedichloropalladium (II)) (30 mg, 0.045 mmol), heated to reflux for 22 hours, quenched with water, and extracted with ethyl acetate. The combined extracts were dried (Na₂SO₄), filtered, and concentrated to provide a mixture of the desired product and Example 193A (0.43 g) which was used without further purification. MS (ESI(+)) m/e 267 (M+H)⁺; ¹H NMR (300 MHz, DMSO-d₆) δ 1.21(t, 3H), 4.26 (q, 2H), 7.14 (t, 1H), 7.34 (t, 1H), 7.41-7.56 (m, 4H), 8.63 (m, 2H), 12.16 (br s, 1H).

### EXAMPLE 212B

### 3-pyridin-4-yl-1H-indole-2-carbohydrazide

A solution of Example 212A (0.43 g, 1.6 mmol) in ethanol (5 mL) was treated with hydrazine hydrate (0.5 mL, 16 mmol), heated to reflux for 18 hours, cooled to room temperature, and filtered. The filter cake was washed with ethanol and dried under vacuum to provide a mixture of the desired product and the 3-unsubstituted product (0.36 g, 90%). MS (ESI(+)) m/e 254 (M+2H)⁺; ¹H NMR (300 MHz, DMSO-d₆) δ 4.03(br s, 2H), 7.14 (t, 1H), 7.27 (t, 1H), 7.47-7.50 (m, 3H), 7.67 (d, 1H), 8.59, (m, 2H), 9.37 (br s, 1H), 11.93(br s, 1H).

### EXAMPLE 212C

### 3-pyridin-4-yl-N′-(1,3-thiazol-2-ylmethylidene)-1H-indole-2-carbohydrazide

A solution of Example 212B (50 mg, 0.20 mmol) and 1,3-thiazole-2-carbaldehyde (33.9 mg, 0.21 mmol) in ethanol (2.5 mL) was heated to 60° C. for 18 hours, cooled to room temperature, and concentrated. The concentrate was dissolved in methylsulfoxide and purified by preparative HPLC to provide the desired product as a mixture of isomers (36.2 mg, 52.6%). MS (ESI(−)) m/e 347, 346 (M−H)⁻; ¹H NMR (300 MHz, DMSO-d₆) δ 7.28 (t, 1H), 7.40 (t, 1H), 7.47 (m, 2H), 7.61 (d, 1H), 7.85 (d, 1H), 7.96, (m, 2H), 8.45 (br s, 1H), 8.77 (m, 2H), 12.27 (br s, 1H), 12.58 (br s, 1H).

### EXAMPLE 213

### 3-pyridin-4-yl-N′-(pyridin-3-ylmethylidene)-1H-indole-2-carbohydrazide

A solution of Example 212B (50 mg, 0.20 mmol) and nicotinaldehyde (32.1 mg, 0.21 mmol) in ethanol (2.5 mL) was heated to 60° C. for 18 hours, cooled to room temperature, and concentrated. The concentrate was dissolved in methylsulfoxide and purified by preparative HPLC with 0.1% trifluoroacetic acid in acetonitrile to provide the desired product (36.6 mg, 54.1%). MS (ESI(−)) m/e 341 (M−H)⁻; ¹H NMR (300 MHz, DMSO-d₆) δ 7.29 (t, 1H), 7.40 (t, 1H), 7.52 (s, 1H), 7.62 (d, 1H), 7.88 (d, 1H), 8.05, (m, 2H), 8.15 (br s, 1H), 8.26 (br s, 1H), 8.52 (br s, 1H), 8.63(br s, 1H), 8.80-8.89 (m, 2H), 12.17 (br s, 1H), 12.66 (br s, 1H).

### EXAMPLE 214

### N′-((4-chlorophenyl)methylidene)-3-pyridin-4-yl-1H-indole-2-carbohydrazide

A solution of Example 212B (50 mg, 0.20 mmol) and 4-chlorobenzaldehyde (42.2 mg, 0.21 mmol) in ethanol (2.5 mL) was heated to 60° C. for 18 hours, cooled to room temperature, and concentrated. The concentrate was dissolved in methylsulfoxide and purified by preparative HPLC with 0.1% trifluoroacetic acid in acetonitrile to provide the desired product (29.2 mg, 40.2%). MS (ESI(−)) m/e 373 (M−H)⁻; ¹H NMR (300 MHz, DMSO-d₆) δ 7.26 (t, 1H), 7.38 (m, 2H), 7.53 (m, 2H), 7.60 (d, 1H), 7.74 (s, 1H), 7.84 (d, 1H), 7.90, (br s, 2H), 8.22 (br s, 1H), 8.60 (br s, 1H), 8.75 (br s, 1H), 11.98 (br s, 1H), 12.49 (br s, 1H).

### EXAMPLE 215

### N′-((4-bromophenyl)methylidene)-3-pyridin-4-yl-1H-indole-2-carbohydrazide

A solution of Example 212B (50 mg, 0.20 mmol) and 4-bromobenzaldehyde (55.5 mg, 0.21 mmol) in ethanol (2.5 mL) was heated to 60° C. for 18 hours, cooled to room temperature, and concentrated. The concentrate was dissolved in methylsulfoxide and purified by preparative HPLC with 0.1% trifluoroacetic acid in acetonitrile to provide the desired product (29.2 mg, 40.2%). MS (ESI(−)) m/e 419 (M−H)⁻; ¹H NMR (300 MHz, DMSO-d₆) δ 7.25 (t, 1H), 7.37 (t, 1H), 7.48 (m, 3H), 7.59 (d, 1H), 7.68 (s, 1H), 7.83 (d, 1H), 7.87, (br s, 2H), 8.20 (br s, 1H), 8.62 (br s, 1H), 8.73 (br s, 1H), 11.95 (br s, 1H), 12.45 (br s, 1H).

### EXAMPLE 216

### N′-(1H-imidazol-2-ylmethylidene)-3-thien-3-yl-1H-indole-2-carbohydrazide

### EXAMPLE 216A

### Ethyl 3-thien-3-yl-1H-indole-2-carboxylate

A solution of Example 193A (2.0 g, 6.3 mmol) in dimethoxyethane (55 mL) was treated with 2N Na₂CO₃ (12.6 mL), 3-thiopheneboronic acid (0.81 g, 6.3 mmol), and (1,1′-bis-(diphenylphosphino)ferrocenedichloropalladium (II)) (33 mg, 0.045 mmol), heated to 84° C. for 18 hours on an Argonaut Quest, cooled to room temperature, and filtered. The filtrate was treated with water and extracted with ethyl acetate. The combined extracts were dried (Na₂SO₄), filtered, and concentrated to provide a 70:30 mixture of product and by-product (2.0 g) which was used without further purification. MS (ESI(+)) m/e 272 (M+H)⁺; ¹H NMR (300 MHz, DMSO-d₆) δ 1.27 (t, 3H), 4.29 (q, 2H), 7.14-7.22 (m, 1H), 7.30-7.36 (m, 1H), 7.41-7.53 (m, 2H), 7.62-7.64 (m, 2H), 7.71 (d, 1H), 12.02 (br s, 1H).

### EXAMPLE 216B

### 3-thien-3-yl-1H-indole-2-carbohydrazide

A solution of Example 216A (1.7 g, 6.3 mmol) in ethanol (15 mL) was treated with hydrazine hydrate (2.0 mL, 63 mmol), heated to reflux for 18 hours, cooled to room temperature, and filtered. The concentrate was washed with ethanol and dried under vacuum to provide an 80:20 mixture of the desired product and 3-unsubstituted product (0.55 g, 34%). MS (ESI(+)) m/e 258 (M+H)⁺; ¹H NMR (300 MHz, DMSO-d₆) δ 4.52 (br s, 2H), 7.00-7.31 (m, 2H), 7.40-7.71 (m, 5H), 9.03 (br s, 1H), 11.80 (br s, 1H).

### EXAMPLE 216C

### N′-(1H-imidazol-2-ylmethylidene)-3-thien-3-yl-1H-indole-2-carbohydrazide

A solution of Example 216B (50 mg, 0.19 mmol) and 1H-imidazole-2-carbaldehyde (27.4 mg, 0.29 mmol) in ethanol (2.5 mL) was heated to 60° C. for 18 hours, cooled to room temperature, and concentrated. The concentrate was dissolved in methylsulfoxide and purified by preparative HPLC with 0.1% trifluoroacetic acid in acetonitrile to provide the desired product as a mixture of isomers (5.8 mg, 8.9%). MS (ESI(+)) m/e 334 (M−H)⁻; ¹H NMR (300 MHz, DMSO-d₆) δ 7.15 (t, 1H), 7.31 (m, 2H), 7.50 (d, 1H), 7.55-7.70 (m, 6H), 7.73 (d, 1H), 8.12 (br s, 1H), 11.93 (br s, 1H).

### EXAMPLE 217

### N′-(pyridin-3-ylmethylidene)-3-thien-3-yl-1H-indole-2-carbohydrazide

A solution of Example 216B (50 mg, 0.19 mmol) and nicotinaldehyde (30.53 mg, 0.29 mmol) in ethanol (2.5 mL) was heated to 60° C. for 18 hours, cooled to room temperature, and concentrated. The concentrate was dissolved in methylsulfoxide and purified by preparative HPLC with 0.1% trifluoroacetic acid in acetonitrile to provide the desired product (17.7 mg, 26.3%). MS (ESI(+)) m/e 346 (M−H)⁻; ¹H NMR (300 MHz, DMSO-d₆) δ 7.14 (t, 1H), 7.29 (t, 1H), 7.32 (s, 1H), 7.49 (d, 1H), 7.70-7.75 (m, 4H), 8.17 (br s, 1H), 8.54 (br s, 1H), 8.73 (br s, 1H), 8.17 (br s, 1H), 11.57 (br s, 1H), 11.82 (br s, 1H).

### EXAMPLE 218

### N′-((4-bromophenyl)methylidene)-3-thien-3-yl-1H-indole-2-carbohydrazide

A solution of Example 216B (50 mg, 0.19 mmol) and 4-bromobenzaldehyde (52.73 mg, 0.29 mmol) in ethanol (2.5 mL) was heated to 60° C. for 18 hours, cooled to room temperature, and concentrated. The concentrate was dissolved in methylsulfoxide and purified by preparative HPLC with 0.1% trifluoroacetic acid in acetonitrile to provide the desired product (10.3 mg, 12.5%). MS (ESI(+)) m/e 425 (M+H)⁺; ¹H NMR (300 MHz, DMSO-d₆) δ 7.07 (t, 1H), 7.23 (t, 1H), 7.32 (s, 1H), 7.47 (d, 1H), 7.66-7.74 (m, 7H), 8.43 (br s, 1H), 11.81 (br s, 1H), 11.96 (br s, 1H).

### EXAMPLE 219

### 3-(3-cyanophenyl)-N′-(1H-imidazol-2-ylmethylidene)-1H-indole-2-carbohydrazide

### EXAMPLE 219A

### Ethyl 3-(3-cyanophenyl)-1H-indole-2-carboxylate

A solution of Example 193A (0.5 g, 1.6 mmol) in dimethoxyethane (20 mL) was treated with 2N Na₂CO₃ (3.2 mL), 3-cyanophenylboronic acid (0.24 g, 1.6 mmol), and (1,1′-bis-(diphenylphosphino)ferrocenedichloropalladium (II)) (8 mg), heated to reflux for 18 hours on an Argonaut Quest, cooled to room temperature, quenched with water, and extracted with ethyl acetate. The combined extracts were dried (Na₂SO₄), filtered, and concentrated to provide the desired product (0.45 g, 94%). MS (ESI(+)) m/e 291 (M+H)⁺; ¹H NMR (300 MHz, DMSO-d₆) δ 1.19(t, 3H), 4.24 (q, 2H), 7.13 (t, 1H), 7.34 (t, 1H), 7.53 (m, 2H), 7.67 (m, 2H), 7.86 (m, 2H), 12.10 (br s, 1H).

### EXAMPLE 219B

### 3-(3-cyanophenyl)-1H-indole-2-carbohydrazide

A solution of Example 219A (0.45 g, 1.6 mmol) in ethanol (5 mL) was treated with hydrazine hydrate (0.5 mL, 16 mmol), heated to reflux for 18 hours, and cooled to room temperature. Water was added and the aqueous layer was extracted with ethyl acetate. The combined extracts were dried (Na₂SO₄), filtered, and concentrated to provide a mixture of the desired product and the 3-unsubstituted product (0.39 g, 90%). MS (ESI(+)) m/e 277 (M+H)⁺; ¹H NMR (300 MHz, DMSO-d₆) δ 4.04 (br s, 2H), 7.12 (t, 1H), 7.27 (t, 1H), 7.47-7.67 (m, 4H), 7.79, (m, 2H), 9.24 (br s, 1H), 11.81 (br s, 1H).

### EXAMPLE 219C

### 3-(3-cyanophenyl)-N′-(1H-imidazol-2-ylmethylidene)-1H-indole-2-carbohydrazide

A solution of Example 219B (50 mg, 0.18 mmol) and 1H-imidazole-2-carbaldehyde (28.9 mg, 0.27 mmol) in ethanol (2.5 mL) was heated to 60° C. for 18 hours, cooled to room temperature, and concentrated. The concentrate was dissolved in methylsulfoxide and purified by preparative HPLC with 0.1% trifluoroacetic acid in acetonitrile to provide the desired product as a mixture of isomers (12.69 mg, 19.6%). MS (ESI(+)) m/e 355 (M+H)⁺; ¹H NMR (300 MHz, DMSO-d₆) δ 7.20 (t, 1H), 7.35 (t, 1H), 7.44 (br s, 1H), 7.54-7.57 (m, 6H), 7.80 (m, 2H), 7.94 (br s, 1H), 8.08 (br s, 1H), 12.11 (br s, 1H).

Following Scheme 1 and the examples above, the following compounds can be prepared:

### EXAMPLE 220

### 4-((((3-isopropyl-1H-indol-2-yl)carbonyl)hydrazono)methyl)benzamide

### EXAMPLE 221

### N′-((4-fluorophenyl)methylidene)-3-vinyl-1H-indole-2-carbohydrazide

### EXAMPLE 222

### 4-((((3-phenyl-1H-indol-2-yl)carbonyl)hydrazono)methyl)benzenesulfonamide

### EXAMPLE 223

### N′-((4-hydroxyphenyl)methylidene)-3-phenyl-1H-indole-2-carbohydrazide

### EXAMPLE 224

### 4-((((3-phenyl-1H-indol-2-yl)carbonyl)hydrazono)methyl)phenyl Sulfamate

### EXAMPLE 225

### N′-((3-chloro-5-(trifluoromethyl)-2-pyridinyl)methylidene)-3-phenyl-1H-indole-2-carbohydrazide

### EXAMPLE 226

### N′-((4-chloro-1,3-thiazol-2-yl)methylidene)-3-isopropyl-1H-indole-2-carbohydrazide

### EXAMPLE 227

### 3-isopropyl-N′-(3-thienylmethylidene)-1H-indole-2-carbohydrazide

### EXAMPLE 228

### 3-isopropyl-N′-(2-thienylmethylidene)-1H-indole-2-carbohydrazide

### EXAMPLE 229

### 3-isopropyl-N′-((3-methyl-2-thienyl)methylidene)-1H-indole-2-carbohydrazide

### EXAMPLE 230

### N′-((5-chloro-2-thienyl)methylidene)-3-isopropyl-1H-indole-2-carbohydrazide

### EXAMPLE 231

### N′-(1-benzofuran-2-ylmethylidene)-3-isopropyl-1H-indole-2-carbohydrazide

It will be evident to one skilled in the art that the present invention is not limited to the forgoing illustrative examples, and that it can be embodied in other specific forms without departing from the essential attributes thereof. It is therefore desired that the examples be considered in all respects as illustrative and not restrictive, reference being made to the appended claims, and all changes which come within the meaning and range of equivalency of the claims and therefore intended to be embraced therein.

What is claimed is:

1. A method of treating a mammal in need of antiproliferative therapy comprising administering to the mammal a therapeutically acceptable amount of a compound of formula (I), 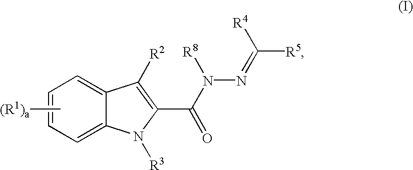
or a therapeutically acceptable salt thereof, wherein
a is 0, 1, 2, 3, or 4;
each R¹ is selected from the group consisting of alkoxy, amino, halo, hydroxy, and nitro;
R² is selected from the group consisting of alkenyl, alkoxy, alkyl, alkylsulfanyl, alkylsulfonyl, alkynyl, aminocarbonyl, Ar¹, arylalkyl, arylsulfanyl, arylsulfonyl, halo, and heterocycle;
R³ is selected from the group consisting of hydrogen, alkyl, and a nitrogen protecting group;
one of R⁴ and R⁵ is independently selected from the group consisting of alkyl, Ar², arylalkyl, cycloalkyl, (cycloalkyl)alkyl, heterocycle, and (heterocycle)alkyl; and the other is selected from the group consisting of hydrogen, and alkyl;
R⁸ is selected from the group consisting of hydrogen, and alkyl;
Ar¹ is an aryl group optionally substituted with one, two, three, four, or five substituents independently selected from the group consisting of alkenyl, alkoxy, alkoxyalkyl, alkyl, amino, cyano, cycloalkyl, (cycloalkyl)alkyl, halo, haloalkoxy, haloalkyl, hydroxy, and nitro; and
Ar² is an aryl group optionally substituted with one, two, three, four, or five substituents independently selected from the group consisting of alkenyl, alkoxy, alkoxyalkyl, alkoxycarbonyl, alkyl, alkylcarbonyl, alkylcarbonyloxy, alkylsulfanyl, alkylsulfonyl, amino, aminocarbonyl, aminosulfonyl, aminosulfonyloxy, cyano, cycloalkyl, (cycloalkyl)alkyl, formyl, halo, haloalkoxy, haloalkyl, heterocycle, hydroxy, hydroxyalkyl, and nitro.

2. The method of claim 1 wherein R³ is hydrogen.

3. The method of claim 2 wherein one of R⁴ and R⁵ is hydrogen and the other is alkyl.

4. The method of claim 3 wherein the compound of formula (I) is selected from the group consisting of
N′-(butylidene)-3-phenyl-1H-indole-2-carbohydrazide; and
N′-(pentylidene)-3-phenyl-1H-indole-2-carbohydrazide.

5. The method of claim 2 wherein one of R⁴ and R⁵ is alkyl and the other is Ar².

6. The method of claim 5 wherein the compound of formula (I) is selected from the group consisting of
N′-(1-(4-cyanophenyl)ethylidene)-3-phenyl-1H-indole-2-carbohydrazide;
N′-(1-(4-cyanophenyl)ethylidene)-3-isopropyl-1H-indole-2-carbohydrazide;
N′-(1-(4-fluorophenyl)ethylidene)-3-isopropyl-1H-indole-2-carbohydrazide;
3-isopropyl-N′-(1-(4-nitrophenyl)ethylidene)-1H-indole-2-carbohydrazide; and
N′-(1-(4-chlorophenyl)ethylidene)-3-isopropyl-1H-indole-2-carbohydrazide.

7. The method of claim 2 wherein one of R⁴ and R⁵ is selected from the group consisting of hydrogen and alkyl, and the other is heterocycle.

8. The method of claim 7 wherein the compound of formula (I) is selected from the group consisting of
3-phenyl-N′-(4-quinolinylmethylidene)-1H-indole-2-carbohydrazide;
3-phenyl-N′-(4-pyridinylmethylidene)-1H-indole-2-carbohydrazide;
3-phenyl-N′-(3-pyridinylmethylidene)-1H-indole-2-carbohydrazide;
3-phenyl-N′-(2-pyridinylmethylidene)-1H-indole-2-carbohydrazide;
N′-((6-methyl-2-pyridinyl)methylidene)-3-phenyl-1H-indole-2-carbohydrazide;
N′-(3-furylmethylidene)-3-phenyl-1H-indole-2-carbohydrazide;
N′-((5-methyl-2-furyl)methylidene)-3-phenyl-1H-indole-2-carbohydrazide;
N′-(1-benzofuran-2-ylmethylidene)-3-phenyl-1H-indole-2-carbohydrazide;
N′-((5-nitro-2-furyl)methylidene)-3-phenyl-1H-indole-2-carbohydrazide;
N′-(2-furylmethylidene)-3-phenyl-1H-indole-2-carbohydrazide;
3-isopropyl-N′-((5-nitro-2-furyl)methylidene)-1H-indole-2-carbohydrazide;
3-isopropyl-N′-((5-methyl-2-furyl)methylidene)-1H-indole-2-carbohydrazide;
3-isopropyl-N′-(3-pyridinylmethylidene)-1H-indole-2-carbohydrazide;
N′-(2-furylmethylidene)-3-isopropyl-1H-indole-2-carbohydrazide;
3-methyl-N′-((5-nitro-2-furyl)methylidene)-1H-indole-2-carbohydrazide;
3-methyl-N′-((5-methyl-2-furyl)methylidene)-1H-indole-2-carbohydrazide;
3-methyl-N′-(3-pyridinylmethylidene)-1H-indole-2-carbohydrazide;
N′-(2-furylmethylidene)-3-methyl-1H-indole-2-carbohydrazide;
3-phenyl-N′-(1,3-thiazol-2-ylmethylidene)-1H-indole-2-carbohydrazide;
N′-((4,5-dimethyl-2-furyl)methylidene)-3-phenyl-1H-indole-2-carbohydrazide;
N′-((5-(4-methylphenyl)-2-furyl)methylidene)-3-phenyl-1H-indole-2-carbohydrazide;
N′-((5-ethyl-2-furyl)methylidene)-3-phenyl-1H-indole-2-carbohydrazide;
(5-((2-((3-phenyl-1H-indol-2-yl)carbonyl)hydrazono)methyl)-2-furyl)methyl acetate;
N′-((5-(4-nitrophenyl)-2-furyl)methylidene)-3-phenyl-1H-indole-2-carbohydrazide;
N′-((4-methyl-1H-imidazol-5-yl)methylidene)-3-phenyl-1H-indole-2-carbohydrazide;
N′-(1H-imidazol-2-ylmethylidene)-3-phenyl-1H-indole-2-carbohydrazide;
N′-((1-methyl-1H-imidazol-2-yl)methylidene)-3-phenyl-1H-indole-2-carbohydrazide;
N′-(1H-imidazol-5-ylmethylidene)-3-phenyl-1H-indole-2-carbohydrazide;
N′-((2-chloro-3-quinolinyl)methylidene)-3-phenyl-1H-indole-2-carbohydrazide;
3-phenyl-N′-(1H-pyrrol-2-ylmethylidene)-1H-indole-2-carbohydrazide;
N′-((1-methyl-1H-pyrrol-2-yl)methylidene)-3-phenyl-1H-indole-2-carbohydrazide;
N′-((4-chloro-1-methyl-1H-pyrazol-3-yl)methylidene)-3-phenyl-1H-indole-2-carbohydrazide;
3-(methylthio)-N′-(1,3-thiazol-2-ylmethylidene)-1H-indole-2-carbohydrazide;
3-(methylsulfonyl)-N′-(1,3-thiazol-2-ylmethylidene)-1H-indole-2-carbohydrazide;
7-chloro-3-methyl-N′-(1,3-thiazol-2-ylmethylidene)-1H-indole-2-carbohydrazide;
4,6-dichloro-3-methyl-N′-(pyridin-3-ylmethylidene)-1H-indole-2-carbohydrazide;
7-chloro-3-phenyl-N′-(pyridin-3-ylmethylidene)-1H-indole-2-carbohydrazide;
7-chloro-N′-(1H-imidazol-2-ylmethylidene)-3-phenyl-1H-indole-2-carbohydrazide;
7-fluoro-3-phenyl-N′-(pyridin-3-ylmethylidene)-1H-indole-2-carbohydrazide;
7-fluoro-N′-(1H-imidazol-2-ylmethylidene)-3-phenyl-1H-indole-2-carbohydrazide;
7-chloro-N′-(1H-imidazol-5-ylmethylidene)-3-phenyl-1H-indole-2-carbohydrazide;
7-chloro-N′-(2-furylmethylidene)-3-phenyl-1H-indole-2-carbohydrazide;
7-chloro-3-phenyl-N′-(1,3-thiazol-2-ylmethylidene)-1H-indole-2-carbohydrazide;
7-fluoro-N′-(1H-imidazol-4-ylmethylidene)-3 -phenyl-1H-indole-2-carbohydrazide;
N′-(2-furylmethylidene)-N-methyl-3-phenyl-1H-indole-2-carbohydrazide;
N′-(1H-imidazol-5-ylmethylidene)-N-methyl-3-phenyl-1H-indole-2-carbohydrazide;
3-phenyl-N′-(quinolin-3-ylmethylidene)-1H-indole-2-carbohydrazide;
N′-((4-chloro-1-methyl-1H-pyrazol-3-yl)methylidene)-3-phenyl-1H-indole-2-carbohydrazide;
3-isopropyl-N′-(quinolin-4-ylmethylidene)-1H-indole-2-carbohydrazide;
3-isopropyl-N′-(isoquinolin-4-ylmethylidene)-1H-indole-2-carbohydrazide;
3-isopropyl-N′-(1,3-thiazol-2-ylmethylidene)-1H-indole-2-carbohydrazide;
N′-(1H-imidazol-5-ylmethylidene)-3-isopropyl-1H-indole-2-carbohydrazide;
N′-(1H-imidazol-2-ylmethylidene)-3-isopropyl-1H-indole-2-carbohydrazide;
N′-((5-ethyl-2-furyl)methylidene)-3-isopropyl-1H-indole-2-carbohydrazide;
N′-(1-benzofuran-2-ylmethylidene)-3-isopropyl-1H-indole-2-carbohydrazide;
N′-(1-(2-furyl)ethylidene)-3-isopropyl-1H-indole-2-carbohydrazide;
3-isopropyl-N′-(1-(5-methyl-2-furyl)ethylidene)-1H-indole-2-carbohydrazide;
N′-(1-(2-furyl)ethylidene)-3-phenyl-1H-indole-2-carbohydrazide;
3-isopropyl-N′-(1-(1,3-thiazol-2-yl)ethylidene)-1H-indole-2-carbohydrazide;
3-phenyl-N′-(1-pyridin-3-ylethylidene)-1H-indole-2-carbohydrazide;
2-(2-(((3-chloro-5-nitropyridin-2-yl)oxy)methyl)phenyl)-2-(methoxyimino)-N-methylethanamide;
3-isopropyl-N′-(1-pyridin-4-ylpropylidene)-1H-indole-2-carbohydrazide;
3-isopropyl-N′-(1H-pyrazol-3-ylmethylidene)-1H-indole-2-carbohydrazide;
3-phenyl-N′-(1H-pyrazol-3-ylmethylidene)-1H-indole-2-carbohydrazide;
N′-((5-ethyl-2-furyl)methylidene)-3-phenyl-1H-indole-2-carbohydrazide;
3-isopropoxy-N′-((5-methyl-2-furyl)methylidene)-1H-indole-2-carbohydrazide;
3-isopropoxy-N′-(1,3-thiazol-2-ylmethylidene)-1H-indole-2-carbohydrazide;
3-methyl-N′-(1,3-thiazol-2-ylmethylidene)-1H-indole-2-carbohydrazide;
N′-(1,3-thiazol-2-ylmethylidene)-3-(4-(trifluoromethoxy)phenyl)-1H-indole-2-carbohydrazide;
3-(3-chlorophenyl)-N′-(1H-imidazol-2-ylmethylidene)-1H-indole-2-carbohydrazide;
3-(3-chlorophenyl)-N′-(1H-imidazol-4-ylmethylidene)-1H-indole-2-carbohydrazide;
3-(3-chlorophenyl)-N′-(1,3-thiazol-2-ylmethylidene)-1H-indole-2-carbohydrazide;
3-(3-chlorophenyl)-N′-(pyridin-3-ylmethylidene)-1H-indole-2-carbohydrazide;
N′-(1H-imidazol-2-ylmethylidene)-3-thien-2-yl-1H-indole-2-carbohydrazide;
N′-(1,3-thiazol-2-ylmethylidene)-3-thien-2-yl-1H-indole-2-carbohydrazide;
N′-(pyridin-3-ylmethylidene)-3-thien-2-yl-1H-indole-2-carbohydrazide;
3-(2-chlorophenyl)-N′-(1H-imidazol-2-ylmethylidene)-1H-indole-2-carbohydrazide;
3-(2-chlorophenyl)-N′-(1H-imidazol-4-ylmethylidene)-1H-indole-2-carbohydrazide;
3-(2-chlorophenyl)-N′-(pyridin-3-ylmethylidene)-1H-indole-2-carbohydrazide;
3-pyridin-4-yl-N′-(1,3-thiazol-2-ylmethylidene)-1H-indole-2-carbohydrazide;
3-pyridin-4-yl-N′-(pyridin-3-ylmethylidene)-1H-indole-2-carbohydrazide;
N′-(1H-imidazol-2-ylmethylidene)-3-thien-3-yl-1H-indole-2-carbohydrazide;
N′-(pyridin-3-ylmethylidene)-3-thien-3-yl-1H-indole-2-carbohydrazide;
3-(3-cyanophenyl)-N′-(1H-imidazol-2-ylmethylidene)-1H-indole-2-carbohydrazide;
N′-(3-chloro-5-(trifluoromethyl)-2-pyridinyl)methylidene)-3-phenyl-1H-indole-2-carbohydrazide;
N′-((4-chloro-1,3-thiazol-2-yl)methylidene)-3-isopropyl-1H-indole-2-carbohydrazide;
3-isopropyl-N′-(3-thienylmethylidene)-1H-indole-2-carbohydrazide;
3-isopropyl-N′-(2-thienylmethylidene)-1H-indole-2-carbohydrazide;
3-isopropyl-N′-((3-methyl-2-thienyl)methylidene)-1H-indole-2-carbohydrazide;
N′-((5-chloro-2-thienyl)methylidene)-3-isopropyl-1H-indole-2-carbohydrazide, and
N′-(1-benzofuran-2-ylmethylidene)-3-isopropyl-1H-indole-2-carbohydrazide.

9. The method of claim 2 wherein one of R⁴ and R⁵ is hydrogen and the other is Ar².

10. The method of claim 9 wherein a is 0.

11. The method of claim 10 wherein R² is alkyl.

12. The method of claim 11 wherein the compound of formula (I) is selected from the group consisting of
N′-((4-methoxyphenyl)methylidene)-3-methyl-1H-indole-2-carbohydrazide;
N′-((4-bromophenyl)methylidene)-3-isopropyl-1H-indole-2-carbohydrazide;
N′-((4-chlorophenyl)methylidene)-3-isopropyl-1H-indole-2-carbohydrazide;
N′-((4-fluorophenyl)methylidene)-3-isopropyl-1H-indole-2-carbohydrazide;
N′-((4-cyanophenyl)methylidene)-3-isopropyl-1H-indole-2-carbohydrazide;
3-isopropyl-N′-(phenylmethylidene)-1H-indole-2-carbohydrazide;
3-isopropyl-N′-((4-nitrophenyl)methylidene)-1H-indole-2-carbohydrazide;
N′-((4-fluorophenyl)methylidene)-3-methyl-1H-indole-2-carbohydrazide;
3-methyl-N′-(phenylmethylidene)-1H-indole-2-carbohydrazide;
N′-((4-bromophenyl)methylidene)-3-methyl-1H-indole-2-carbohydrazide;
N′-((4-cyanophenyl)methylidene)-3-methyl-1H-indole-2-carbohydrazide;
N′-((4-(difluoromethoxy)phenyl)methylidene)-3-methyl-1H-indole-2-carbohydrazide;
3-isopropyl-N′-(1-naphthylmethylidene)-1H-indole-2-carbohydrazide;
N′-((3,4-dichlorophenyl)methylidene)-3-isopropyl-1H-indole-2-carbohydrazide;
N′-((2,4-dichlorophenyl)methylidene)-3-isopropyl-1H-indole-2-carbohydrazide;
N′-((4-iodophenyl)methylidene)-3-isopropyl-1H-indole-2-carbohydrazide;
N′-((4-hydroxyphenyl)methylidene)-3-methyl-1H-indole-2-carbohydrazide;
N′-((4-iodophenyl)methylidene)-3-methyl-1H-indole-2-carbohydrazide; and
4-((((3-isopropyl-1H-indol-2-yl)carbonyl)hydrazono)methyl)benzamide.

13. The method of claim 10 wherein R² is Ar¹.

14. The method of claim 13 wherein the compound of formula (I) is selected from the group consisting of
N′-((4-methoxyphenyl)methylidene)-3-phenyl-1H-indole-2-carbohydrazide;
N′-((4-bromophenyl)methylidene)-3-phenyl-1H-indole-2-carbohydrazide;
3-phenyl-N′-((4-(trifluoromethoxy)phenyl)methylidene)-1H-indole-2-carbohydrazide;
N′-((4-(difluoromethoxy)phenyl)methylidene)-3-phenyl-1H-indole-2-carbohydrazide;
N′-((3-bromophenyl)methylidene)-3-phenyl-1H-indole-2-carbohydrazide,;
N′-((4-(dihydroxyamino)phenyl)methylidene)-3-phenyl-1H-indole-2-carbohydrazide;
3-phenyl-N′-(phenylmethylidene)-1H-indole-2-carbohydrazide;
N′-((3-cyanophenyl)methylidene)-3-phenyl-1H-indole-2-carbohydrazide;
N′-((4-cyanophenyl)methylidene)-3-phenyl-1H-indole-2-carbohydrazide;
N-(4-((2-((3-phenyl-1H-indol-2-yl)carbonyl)hydrazono)methyl)phenyl)acetamide;
N′-((4-(diethylamino)phenyl)methylidene)-3-phenyl-1H-indole-2-carbohydrazide;
N′-((4-isopropylphenyl)methylidene)-3-phenyl-1H-indole-2-carbohydrazide;
N′-((3-nitrophenyl)methylidene)-3-phenyl-1H-indole-2-carbohydrazide;
3-phenyl-N′-((4-(1-pyrrolidinyl)phenyl)methylidene)-1H-indole-2-carbohydrazide;
N′-((4-(methylsulfonyl)phenyl)methylidene)-3-phenyl-1H-indole-2-carbohydrazide;
N′-((4-chlorophenyl)methylidene)-3-phenyl-1H-indole-2-carbohydrazide;
N′-(1-naphthylmethylidene)-3-phenyl-1H-indole-2-carbohydrazide;
3-phenyl-N′-((4-(trifluoromethyl)phenyl)methylidene)-1H-indole-2-carbohydrazide;
methyl 4-((((3-phenyl-1H-indol-2-yl)carbonyl)hydrazono)methyl)benzoate;
N′-((4-bromophenyl)methylidene)-3-(4-fluorophenyl)-1H-indole-2-carbohydrazide;
N′-((4-chlorophenyl)methylidene)-3-(4-fluorophenyl)-1H-indole-2-carbohydrazide;
3-(4-fluorophenyl)-N′-((4-nitrophenyl)methylidene)-1H-indole-2-carbohydrazide;
N′-((4-cyanophenyl)methylidene)-3-(4-fluorophenyl)-1H-indole-2-carbohydrazide;
3-(4-chlorophenyl)-N′-((4-cyanophenyl)methylidene)-1H-indole-2-carbohydrazide;
N′-((4-bromophenyl)methylidene)-3-(4-chlorophenyl)-1H-indole-2-carbohydrazide;
3-(4-chlorophenyl)-N′-((4-chlorophenyl)methylidene)-1H-indole-2-carbohydrazide;
3-(4-chlorophenyl)-N′-((4-nitrophenyl)methylidene)-1H-indole-2-carbohydrazide;
N′-((4-bromo-3,5-dimethoxyphenyl)methylidene)-3-phenyl-1H-indole-2-carbohydrazide;
N′-((3,4-dichlorophenyl)methylidene)-3-phenyl-1H-indole-2-carbohydrazide;
N′-((4-bromo-2-fluorophenyl)methylidene)-3-phenyl-1H-indole-2-carbohydrazide;
N′-((2,4-dichlorophenyl)methylidene)-3-phenyl-1H-indole-2-carbohydrazide;
N-((4-chloro-3-nitrophenyl)methylidene)-3-phenyl-1H-indole-2-carbohydrazide;
N′-((4-cyanophenyl)methylidene)-3-(3,4-dimethylphenyl)-1H-indole-2-carbohydrazide;
N′-((4-chlorophenyl)methylidene)-3-(3,4-dimethylphenyl)-1H-indole-2-carbohydrazide;
3-(3,4-dimethylphenyl)-N′-((4-nitrophenyl)methylidene)-1H-indole-2-carbohydrazide;
3-(3,4-dimethylphenyl)-N′-((4-fluorophenyl)methylidene)-1H-indole-2-carbohydrazide;
N′-((4-cyanophenyl)methylidene)-N-methyl-3-phenyl-1H-indole-2-carbohydrazide;
N′-((4-bromophenyl)methylidene)-N-methyl-3-phenyl-1H-indole-2-carbohydrazide;
N′-((4-iodophenyl)methylidene)-3-phenyl-1H-indole-2-carbohydrazide;
N′-((4-chlorophenyl)methylidene)-3-(4-(trifluoromethoxy)phenyl)-1H-indole-2-carbohydrazide;
N′-((4-bromophenyl)methylidene)-3-(4-(trifluoromethoxy)phenyl)-1H-indole-2-carbohydrazide;
N′-((4-bromophenyl)methylidene)-3-(3-chlorophenyl)-1H-indole-2-carbohydrazide;
N′-((4-bromophenyl)methylidene)-3-(4-(dimethylamino)phenyl)-1H-indole-2-carbohydrazide;
3-(2-chlorophenyl)-N′-((4-chlorophenyl)methylidene)-1H-indole-2-carbohydrazide;
N′-((4-bromophenyl)methylidene)-3-(2-chlorophenyl)-1H-indole-2-carbohydrazide;
4-((((3-phenyl-1H-indol-2-yl)carbonyl)hydrazono)methyl)benzenesulfonamide;
N′-((4-hydroxyphenyl)methylidene)-3-phenyl-1H-indole-2-carbohydrazide; and
4-((((3-phenyl-1H-indol-2-yl)carbonyl)hydrazono)methyl)phenyl sulfamate.

15. The method of claim 10 wherein R² is selected from the group consisting of alkenyl, alkoxy, alkylsulfanyl, alkylsulfonyl, aminocarbonyl, arylalkyl, arylsulfanyl, arylsulfonyl, halo, and heterocycle.

16. The method of claim 15 wherein the compound of formula (I) is selected from the group consisting of
N′-((4-chlorophenyl)methylidene)-3-(phenylsulfonyl)-1H-indole-2-carbohydrazide;
N′-((4-bromophenyl)methylidene)-3-(phenylsulfonyl)-1H-indole-2-carbohydrazide;
3-benzyl-N′-((4-chlorophenyl)methylidene)-1H-indole-2-carbohydrazide;
3-benzyl-N′-((4-bromophenyl)methylidene)-1H-indole-2-carbohydrazide;
2-((2-(4-chlorobenzylidene)hydrazino)carbonyl)-N,N-diethyl-1H-indole-3-carboxamide;
2-((2-(4-chlorobenzylidene)hydrazino)carbonyl)-N,N-dimethyl-1H-indole-3-carboxamide;
2-((2-(4-chlorobenzylidene)hydrazino)carbonyl)-N-phenyl-1H-indole-3-carboxamide;
N′-((4-chlorophenyl)methylidene)-3-(methylthio)-1H-indole-2-carbohydrazide;
N′-((4-chlorophenyl)methylidene)-3-(methylsulfonyl)-1H-indole-2-carbohydrazide;
3-(methylsulfonyl)-N′-(2-naphthylmethylidene)-1H-indole-2-carbohydrazide;
N′-((4-chlorophenyl)methylidene)-3-methoxy-1H-indole-2-carbohydrazide;
3-bromo-N′-((4-chlorophenyl)methylidene)-1H-indole-2-carbohydrazide;
3-(methylsulfonyl)-N′-(1-naphthylmethylidene)-1H-indole-2-carbohydrazide;
N,N-dimethyl-2-((2-(2-naphthylmethylene)hydrazino)carbonyl)-1H-indole-3-carboxamide;
N′-((4-chlorophenyl)methylidene)-3-isopropoxy-1H-indole-2-carbohydrazide;
N′-((4-cyanophenyl)methylidene)-3-isopropoxy-1H-indole-2-carbohydrazide;
3-chloro-N′-(phenylmethylidene)-1H-indole-2-carbohydrazide;
3-chloro-N′-((4-chlorophenyl)methylidene)-1H-indole-2-carbohydrazide;
3-chloro-N′-((4-cyanophenyl)methylidene)-1H-indole-2-carbohydrazide;
3-chloro-N′-((4-(difluoromethoxy)phenyl)methylidene)-1H-indole-2-carbohydrazide;
N′-((4-chlorophenyl)methylidene)-3-thien-2-yl-1H-indole-2-carbohydrazide;
N′-((4-bromophenyl)methylidene)-3-thien-2-yl-1H-indole-2-carbohydrazide;
N′-((4-chlorophenyl)methylidene)-3-pyridin-4-yl-1H-indole-2-carbohydrazide;
N′-((4-bromophenyl)methylidene)-3-pyridin-4-yl-1H-indole-2-carbohydrazide;
N′-((4-bromophenyl)methylidene)-3-thien-3-yl-1H-indole-2-carbohydrazide; and
N′-((4-fluorophenyl)methylidene)-3-vinyl-1H-indole-2-carbohydrazide.

17. The method of claim 9 wherein a is 1.

18. The method of claim 17 wherein the compound of formula (I) is selected from the group consisting of
N′-((4-bromophenyl)methylidene)-5-fluoro-3-phenyl-1H-indole-2-carbohydrazide;
N′-((4-chlorophenyl)methylidene)-5-fluoro-3-phenyl-1H-indole-2-carbohydrazide;
5-fluoro-N′-((4-fluorophenyl)methylidene)-3-phenyl-1H-indole-2-carbohydrazide;
N′-((4-bromophenyl)methylidene)-5-methoxy-3-phenyl-1H-indole-2-carbohydrazide;
N′-((4-chlorophenyl)methylidene)-5-methoxy-3-phenyl-1H-indole-2-carbohydrazide;
N′-((4-fluorophenyl)methylidene)-5-methoxy-3-phenyl-1H-indole-2-carbohydrazide;
5-bromo-N′-((4-bromophenyl)methylidene)-3-phenyl-1H-indole-2-carbohydrazide;
5-bromo-N′-((4-chlorophenyl)methylidene)-3-phenyl-1H-indole-2-carbohydrazide;
5-bromo-N′-((4-fluorophenyl)methylidene)-3-phenyl-1H-indole-2-carbohydrazide;
N′-((4-cyanophenyl)methylidene)-5-fluoro-3-phenyl-1H-indole-2-carbohydrazide;
N′-((4-cyanophenyl)methylidene)-5-methoxy-3-phenyl-1H-indole-2-carbohydrazide;
5-bromo-N′-((4-cyanophenyl)methylidene)-3-phenyl-1H-indole-2-carbohydrazide;
5-fluoro-3-phenyl-N′-(phenylmethylidene)-1H-indole-2-carbohydrazide;
5-methoxy-3-phenyl-N′-(phenylmethylidene)-1H-indole-2-carbohydrazide;
5-bromo-3-phenyl-N′-(phenylmethylidene)-1H-indole-2-carbohydrazide;
5-fluoro-N′-((4-nitrophenyl)methylidene)-3-phenyl-1H-indole-2-carbohydrazide;
5-methoxy-N′-((4-nitrophenyl)methylidene)-3-phenyl-1H-indole-2-carbohydrazide;
5-bromo-N′-((4-nitrophenyl)methylidene)-3-phenyl-1H-indole-2-carbohydrazide;
7-chloro-N′-((4-chlorophenyl)methylidene)-3-methyl-1H-indole-2-carbohydrazide;
7-chloro-N′-((4-methoxyphenyl)methylidene)-3-methyl-1H-indole-2-carbohydrazide;
N′-((4-chlorophenyl)methylidene)-6-methoxy-3-methyl-1H-indole-2-carbohydrazide;
6-chloro-N′-((4-chlorophenyl)methylidene)-3-methyl-1H-indole-2-carbohydrazide;
6-chloro-N′-((4-methoxyphenyl)methylidene)-3-methyl-1H-indole-2-carbohydrazide;
6-chloro-N′-((4-cyanophenyl)methylidene)-3-methyl-1H-indole-2-carbohydrazide;
4-chloro-N′-((4-chlorophenyl)methylidene)-3-methyl-1H-indole-2-carbohydrazide;
4-chloro-N′-((4-methoxyphenyl)methylidene)-3-methyl-1H-indole-2-carbohydrazide;
4-chloro-N′-((4-cyanophenyl)methylidene)-3-methyl-1H-indole-2-carbohydrazide;
N′-((4-bromophenyl)methylidene)-6-methoxy-3-methyl-1H-indole-2-carbohydrazide;
6-methoxy-N′-((4-methoxyphenyl)methylidene)-3-methyl-1H-indole-2-carbohydrazide;
N′-((4-bromophenyl)methylidene)-4-chloro-3-methyl-1H-indole-2-carbohydrazide;
N′-((4-bromophenyl)methylidene)-7-chloro-3-methyl-1H-indole-2-carbohydrazide;
7-chloro-N′-((4-chlorophenyl)methylidene)-3-phenyl-1H-indole-2-carbohydrazide;
7-chloro-N′-((4-cyanophenyl)methylidene)-3-phenyl-1H-indole-2-carbohydrazide;
N′-((4-chlorophenyl)methylidene)-7-fluoro-3-phenyl-1H-indole-2-carbohydrazide;
N′-((4-cyanophenyl)methylidene)-7-fluoro-3-phenyl-1H-indole-2-carbohydrazide;
N′-((4-bromophenyl)methylidene)-7-chloro-3-phenyl-1H-indole-2-carbohydrazide; and
N′-((4-bromophenyl)methylidene)-7-fluoro-3-phenyl-1H-indole-2-carbohydrazide.

19. The method of claim 9 wherein a is 2.

20. The method of claim 19 wherein the compound of formula (I) is selected from the group consisting of
4,6-dichloro-N′-((4-chlorophenyl)methylidene)-3-methyl-1H-indole-2-carbohydrazide;
4,6-dichloro-N′-((4-cyanophenyl)methylidene)-3-methyl-1H-indole-2-carbohydrazide; and
N′-((4-bromophenyl)methylidene)-4,6-dichloro-3-methyl-1H-indole-2-carbohydrazide.

21. The method of claim 1 wherein the antiproliferative therapy is antiangiogenic therapy.

22. A compound of formula (II), 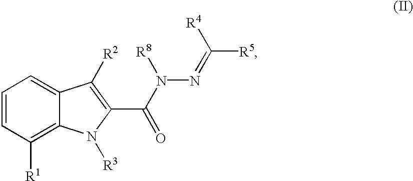
or a therapeutically acceptable salt thereof, wherein
R¹ is selected from the group consisting of hydrogen, alkoxy, amino, halo, and hydroxy;
R² is selected from the group consisting of alkenyl, alkoxy, alkyl, alkylsulfanyl, alkylsulfonyl, alkynyl, aminocarbonyl, Ar³, arylalkyl, arylsulfanyl, arylsulfonyl, halo, and heterocycle;
R³ is selected from the group consisting of hydrogen, alkyl, and a nitrogen protecting group;
one of R⁴ and R⁵ is independently selected from the group consisting of alkyl, Ar⁴, arylalkyl, cycloalkyl, (cycloalkyl)alkyl, heterocycle, and (heterocycle)alkyl; and the other is selected from the group consisting of hydrogen, and alkyl;
R⁸ is selected from the group consisting of hydrogen and alkyl;
Ar³is an aryl group optionally substituted with one, two, three, four, or five substituents independently selected from the group consisting of alkenyl, alkoxy, alkoxyalkyl, alkyl, amino, cyano, cycloalkyl, (cycloalkyl)alkyl, halo, haloalkoxy, haloalkyl, hydroxy, and nitro; and
Ar⁴ is an aryl group optionally substituted with one, two, three, four, or five substituents independently selected from the group consisting of alkoxy, alkoxycarbonyl, alkyl, alkylsulfonyl, aminocarbonyl, aminosulfonyl, aminosulfonyloxy, cyano, halo, haloalkoxy, heterocycle, and hydroxy; with the proviso that when Ar³ is unsubstituted, Ar⁴ is substituted.

23. The compound of claim 22 wherein R³ is hydrogen.

24. The compound of claim 23 wherein one of R⁴ and R ⁵ is hydrogen and the other is alkyl.

25. The compound of claim 24 selected from the group consisting of
N′-(butylidene)-3-phenyl-1H-indole-2-carbohydrazide; and
N′-(pentylidene)-3-phenyl-1H-indole-2-carbohydrazide.

26. The compound of claim 23 wherein one of R⁴ and R⁵ is alkyl and the other is Ar⁴.

27. The compound of claim 26 selected from the group consisting of
N′-(1-(4-cyanophenyl)ethylidene)-3-phenyl-1H-indole-2-carbohydrazide;
N′-(1-(4-cyanophenyl)ethylidene)-3-isopropyl-1H-indole-2-carbohydrazide;
N′-(1-(4-fluorophenyl)ethylidene)-3-isopropyl-1H-indole-2-carbohydrazide; and
N′-(1-(4-chlorophenyl)ethylidene)-3-isopropyl-1H-indole-2-carbohydrazide.

28. The compound of claim 23 wherein one of R⁴ and R⁵ is selected from the group consisting of hydrogen and alkyl, and the other is heterocycle.

29. The compound of claim 28 selected from the group consiting of
3-phenyl-N′-(4-quinolinylmethylidene)-1H-indole-2-carbohydrazide;
3-phenyl-N′-(4-pyridinylmethylidene)-1H-indole-2-carbohydrazide;
3-phenyl-N′-(3-pyridinylmethylidene)-1H-indole-2-carbohydrazide;
3-phenyl-N′-(2-pyridinylmethylidene)-1H-indole-2-carbohydrazide;
N′-((6-methyl-2-pyridinyl)methylidene)-3-phenyl-1H-indole-2-carbohydrazide;
N′-(3-furylmethylidene)-3-phenyl-1H-indole-2-carbohydrazide;
N′-((5-methyl-2-furyl)methylidene)-3-phenyl-1H-indole-2-carbohydrazide;
N′-(1-benzofuran-2-ylmethylidene)-3-phenyl-1H-indole-2-carbohydrazide;
N′-((5-nitro-2-furyl)methylidene)-3-phenyl-1H-indole-2-carbohydrazide;
N′-(2-furylmethylidene)-3-phenyl-1H-indole-2-carbohydrazide;
3-isopropyl-N′-((5-nitro-2-furyl)methylidene)-1H-indole-2-carbohydrazide;
3-isopropyl-N′-((5-methyl-2-furyl)methylidene)-1H-indole-2-carbohydrazide;
3-isopropyl-N′-(3-pyridinylmethylidene)-1H-indole-2-carbohydrazide;
N′-(2-furylmethylidene)-3-isopropyl-1H-indole-2-carbohydrazide;
3-methyl-N′-((5-nitro-2-furyl)methylidene)-1H-indole-2-carbohydrazide;
3-methyl-N′-((5-methyl-2-furyl)methylidene)-1H-indole-2-carbohydrazide;
3-methyl-N′-(3-pyridinylmethylidene)-1H-indole-2-carbohydrazide;
N′-(2-furylmethylidene)-3-methyl-1H-indole-2-carbohydrazide;
3-phenyl-N′-(1,3-thiazol-2-ylmethylidene)-1H-indole-2-carbohydrazide;
N′-((4,5-dimethyl-2-furyl)methylidene)-3-phenyl-1H-indole-2-carbohydrazide;
N′-((5-(4-methylphenyl)-2-furyl)methylidene)-3-phenyl-1H-indole-2-carbohydrazide;
N′-((5-ethyl-2-furyl)methylidene)-3-phenyl-1H-indole-2-carbohydrazide;
(5-((2-((3-phenyl-1H-indol-2-yl)carbonyl)hydrazono)methyl)-2-furyl)methyl acetate;
N′-((5-(4-nitrophenyl)-2-furyl)methylidene)-3-phenyl-1H-indole-2-carbohydrazide;
N′-((4-methyl-1H-imidazol-5-yl)methylidene)-3-phenyl-1H-indole-2-carbohydrazide;
N′-(1H-imidazol-2-ylmethylidene)-3-phenyl-1H-indole-2-carbohydrazide;
N′-((1-methyl-1H-imidazol-2-yl)methylidene)-3-phenyl-1H-indole-2-carbohydrazide;
N′-(1H-imidazol-5-ylmethylidene)-3-phenyl-1H-indole-2-carbohydrazide;
N′-((2-chloro-3-quinolinyl)methylidene)-3-phenyl-1H-indole-2-carbohydrazide;
3-phenyl-N′-(1H-pyrrol-2-ylmethylidene)-1H-indole-2-carbohydrazide;
N′-((1-methyl-1H-pyrrol-2-yl)methylidene)-3-phenyl-1H-indole-2-carbohydrazide;
N′-((4-chloro-1-methyl-1H-pyrazol-3-yl)methylidene)-3-phenyl-1H-indole-2-carbohydrazide;
3-(methylthio)-N′-(1,3-thiazol-2-ylmethylidene)-1H-indole-2-carbohydrazide;
3-(methylsulfonyl)-N′-(1,3-thiazol-2-ylmethylidene)-1H-indole-2-carbohydrazide;
7-chloro-3-methyl-N′-(1,3-thiazol-2-ylmethylidene)-1H-indole-2-carbohydrazide;
7-chloro-3-phenyl-N′-(pyridin-3-ylmethylidene)-1H-indole-2-carbohydrazide;
7-chloro-N′-(1H-imidazol-2-ylmethylidene)-3-phenyl-1H-indole-2-carbohydrazide;
7-fluoro-3-phenyl-N′-(pyridin-3-ylmethylidene)-1H-indole-2-carbohydrazide;
7-fluoro-N′-(1H-imidazol-2-ylmethylidene)-3-phenyl- 1H-indole-2-carbohydrazide;
7-chloro-N′-(1H-imidazol-5-ylmethylidene)-3-phenyl-1H-indole-2-carbohydrazide;
7-chloro-N′-(2-furylmethylidene)-3-phenyl-1H-indole-2-carbohydrazide;
7-chloro-3-phenyl-N′-(1,3-thiazol-2-ylmethylidene)-1H-indole-2-carbohydrazide;
7-fluoro-N′-(1H-imidazol-4-ylmethylidene)-3-phenyl-1H-indole-2-carbohydrazide;
N′-(2-furylmethylidene)-N-methyl-3-phenyl-1H-indole-2-carbohydrazide;
N′-(1H-imidazol-5-ylmethylidene)-N-methyl-3-phenyl-1H-indole-2-carbohydrazide;
3-phenyl-N′-(quinolin-3-ylmethylidene)-1H-indole-2-carbohydrazide;
N′-((4-chloro-1-methyl-1H-pyrazol-3-yl)methylidene)-3-phenyl-1H-indole-2-carbohydrazide;
3-isopropyl-N ′-(quinolin-4-ylmethylidene)-1H-indole-2-carbohydrazide;
3-isopropyl-N′-(isoquinolin-4-ylmethylidene)-1H-indole-2-carbohydrazide;
3-isopropyl-N′-(1,3-thiazol-2-ylmethylidene)-1H-indole-2-carbohydrazide;
N′-(1H-imidazol-5-ylmethylidene)-3-isopropyl-1H-indole-2-carbohydrazide;
N′-(1H-imidazol-2-ylmethylidene)-3-isopropyl-1H-indole-2-carbohydrazide;
N′-((5-ethyl-2-furyl)methylidene)-3-isopropyl-1H-indole-2-carbohydrazide;
N′-(1-benzofuran-2-ylmethylidene)-3-isopropyl-1H-indole-2-carbohydrazide;
N′-(1-(2-furyl)ethylidene)-3-isopropyl-1H-indole-2-carbohydrazide;
3-isopropyl-N′-(1-(5-methyl-2-furyl)ethylidene)-1H-indole-2-carbohydrazide;
N′-(1-(2-furyl)ethylidene)-3-phenyl-1H-indole-2-carbohydrazide;
3-isopropyl-N′-(1-(1,3-thiazol-2-yl)ethylidene)-1H-indole-2-carbohydrazide;
3-phenyl-N′-(1-pyridin-3-ylethylidene)-1H-indole-2-carbohydrazide;
2-(2-(((3-chloro-5-nitropyridin-2-yl)oxy)methyl)phenyl)-2-(methoxyimino)-N-methylethanamide;
3-isopropyl-N′-(1-pyridin-4-ylpropylidene)-1H-indole-2-carbohydrazide;
3-isopropyl-N′-(1H-pyrazol-3-ylmethylidene)-1H-indole-2-carbohydrazide;
3-phenyl-N′-(1H-pyrazol-3-ylmethylidene)-1H-indole-2-carbohydrazide;
N′-((5-ethyl-2-furyl)methylidene)-3-phenyl-1H-indole-2-carbohydrazide;
3-isopropoxy-N′-((5-methyl-2-furyl)methylidene)-1H-indole-2-carbohydrazide;
3-isopropoxy-N′-(1,3-thiazol-2-ylmethylidene)-1H-indole-2-carbohydrazide;
3-methyl-N′-(1,3-thiazol-2-ylmethylidene)-1H-indole-2-carbohydrazide;
N′-(1,3-thiazol-2-ylmethylidene)-3-(4-(trifluoromethoxy)phenyl)-1H-indole-2-carbohydrazide;
3-(3-chlorophenyl)-N′-(1H-imidazol-2-ylmethylidene)-1H-indole-2-carbohydrazide;
3-(3-chlorophenyl)-N′-(1H-imidazol-4-ylmethylidene)-1H-indole-2-carbohydrazide;
3-(3-chlorophenyl)-N′-(1,3-thiazol-2-ylmethylidene)-1H-indole-2-carbohydrazide;
3-(3-chlorophenyl)-N′-(pyridin-3-ylmethylidene)-1H-indole-2-carbohydrazide;
N′-(1H-imidazol-2-ylmethylidene)-3-thien-2-yl-1H-indole-2-carbohydrazide;
N′-(1,3-thiazol-2-ylmethylidene)-3-thien-2-yl-1H-indole-2-carbohydrazide;
N′-(pyridin-3-ylmethylidene)-3-thien-2-yl-1H-indole-2-carbohydrazide;
3-(2-chlorophenyl)-N′-(1H-imidazol-2-ylmethylidene)-1H-indole-2-carbohydrazide;
3-(2-chlorophenyl)-N′-(1H-imidazol-4-ylmethylidene)-1H-indole-2-carbohydrazide;
3-(2-chlorophenyl)-N′-(pyridin-3-ylmethylidene)-1H-indole-2-carbohydrazide;
3-pyridin4-yl-N′-(1,3-thiazol2-ylmethylidene)-1H-indole-2-carbohydrazide;
3-pyridin-4-yl-N′-(pyridin-3-ylmethylidene)-1H-indole-2-carbohydrazide;
N′-(1H-imidazol-2-ylmethylidene)-3-thien-3-yl-1H-indole-2-carbohydrazide;
N′-(pyridin-3-ylmethylidene)-3-thien-3-yl-1H-indole-2-carbohydrazide;
3-(3-cyanophenyl)-N′-(1H-imidazol-2-ylmethylidene)-1H-indole-2-carbohydrazide;
N′-(3-chloro-5-(trifluoromethyl)-2-pyridinyl)methylidene)-3-phenyl-1H-indole-2-carbohydrazide;
N′-((4-chloro-1,3-thiazol-2-yl)methylidene)-3-isopropyl-1H-indole-2-carbohydrazide;
3-isopropyl-N′-(3-thienylmethylidene)-1H-indole-2-carbohydrazide;
3-isopropyl-N′-(2-thienylmethylidene)-1H-indole-2-carbohydrazide;
3-isopropyl-N′-((3-methyl-2-thienyl)methylidene)-1H-indole-2-carbohydrazide;
N′-((5-chloro-2-thienyl)methylidene)-3-isopropyl-1H-indole-2-carbohydrazide, and
N′-(1-benzofuran-2-ylmethylidene)-3-isopropyl-1H-indole-2-carbohydrazide.

30. The compound of claim 23 wherein one of R⁴ and R⁵ is hydrogen and the other is Ar⁴.

31. The compound of claim 30 wherein R¹ is hydrogen.

32. The compound of claim 31 wherein R² is alkyl.

33. The compound of claim 32 selected from the group consisting of
N′-((4-methoxyphenyl)methylidene)-3-methyl-1H-indole-2-carbohydrazide;
N′-((4-bromophenyl)methylidene)-3-isopropyl-1H-indole-2-carbohydrazide;
N′-((4-chlorophenyl)methylidene)-3-isopropyl-1H-indole-2-carbohydrazide;
N′-((4-fluorophenyl)methylidene)-3-isopropyl-1H-indole-2-carbohydrazide;
N′-((4-cyanophenyl)methylidene)-3-isopropyl-1H-indole-2-carbohydrazide;
3-isopropyl-N′-(phenylmethylidene)-1H-indole-2-carbohydrazide;
N′-((4-fluorophenyl)methylidene)-3-methyl-1H-indole-2-carbohydrazide;
3-methyl-N′-(phenylmethylidene)-1H-indole-2-carbohydrazide;
N′-((4-bromophenyl)methylidene)-3-methyl-1H-indole-2-carbohydrazide;
N′-((4-cyanophenyl)methylidene)-3-methyl-1H-indole-2-carbohydrazide;
N′-((4-(difluoromethoxy)phenyl)methylidene)-3-methyl-1H-indole-2-carbohydrazide;
3-isopropyl-N′-(1-naphthylmethylidene)-1H-indole-2-carbohydrazide;
N′-((3,4-dichlorophenyl)methylidene)-3-isopropyl-1H-indole-2-carbohydrazide;
N′-((2,4-dichlorophenyl)methylidene)-3-isopropyl-1H-indole-2-carbohydrazide;
N′-((4-iodophenyl)methylidene)-3-isopropyl-1H-indole-2-carbohydrazide;
N′-((4-hydroxyphenyl)methylidene)-3-methyl-1H-indole-2-carbohydrazide;
N′-((4-iodophenyl)methylidene)-3-methyl-1H-indole-2-carbohydrazide; and
4-((((3-isopropyl-1H-indol-2-yl)carbonyl)hydrazono)methyl)benzamide.

34. The compound of claim 31 wherein R² is Ar³.

35. The compound of claim 34 selected from the group consisting of
N′-((4-methoxyphenyl)methylidene)-3-phenyl-1H-indole-2-carbohydrazide;
N′-((4-bromophenyl)methylidene)-3-phenyl-1H-indole-2-carbohydrazide;
3-phenyl-N′-((4-(trifluoromethoxy)phenyl)methylidene)-1H-indole-2-carbohydrazide;
N′-((4-(difluoromethoxy)phenyl)methylidene)-3-phenyl-1H-indole-2-carbohydrazide;
N′-((3-bromophenyl)methylidene)-3-phenyl-1H-indole-2-carbohydrazide;
N′-((3-cyanophenyl)methylidene)-3-phenyl-1H-indole-2-carbohydrazide;
N′-((4-cyanophenyl)methylidene)-3-phenyl-1H-indole-2-carbohydrazide;
N′-((4-isopropylphenyl)methylidene)-3-phenyl-1H-indole-2-carbohydrazide;
3-phenyl-N′-((4-(1-pyrrolidinyl)phenyl)methylidene)-1H-indole-2-carbohydrazide;
N′-((4-(methylsulfonyl)phenyl)methylidene)-3-phenyl-1H-indole-2-carbohydrazide;
N′-((4-chlorophenyl)methylidene)-3-phenyl-1H-indole-2-carbohydrazide;
methyl 4-((((3-phenyl-1H-indol-2-yl)carbonyl)hydrazono)methyl)benzoate;
N′-((4-bromophenyl)methylidene)-3-(4-fluorophenyl)-1H-indole-2-carbohydrazide;
N′-((4-chlorophenyl)methylidene)-3-(4-fluorophenyl)-1H-indole-2-carbohydrazide;
N′-((4-cyanophenyl)methylidene)-3-(4-fluorophenyl)-1H-indole-2-carbohydrazide;
3-(4-chlorophenyl)-N′-((4-cyanophenyl)methylidene)-1H-indole-2-carbohydrazide;
N′-((4-bromophenyl)methylidene)-3-(4-chlorophenyl)-1H-indole-2-carbohydrazide;
3-(4-chlorophenyl)-N′-((4-chlorophenyl)methylidene)-1H-indole-2-carbohydrazide;
N′-((4-bromo-3,5-dimethoxyphenyl)methylidene)-3-phenyl-1H-indole-2-carbohydrazide;
N′-((3,4-dichlorophenyl)methylidene)-3-phenyl-1H-indole-2-carbohydrazide;
N′-((4-bromo-2-fluorophenyl)methylidene)-3-phenyl-1H-indole-2-carbohydrazide;
N′-((2,4-dichlorophenyl)methylidene)-3-phenyl-1H-indole-2-carbohydrazide;
N′-((4-cyanophenyl)methylidene)-3-(3,4-dimethylphenyl)-1H-indole-2-carbohydrazide;
N′-((4-chlorophenyl)methylidene)-3-(3,4-dimethylphenyl)-1H-indole-2-carbohydrazide;
3-(3,4-dimethylphenyl)-N′-((4-fluorophenyl)methylidene)-1H-indole-2-carbohydrazide;
N′-((4-cyanophenyl)methylidene)-N-methyl-3-phenyl-1H-indole-2-carbohydrazide;
N′-((4-bromophenyl)methylidene)-N-methyl-3-phenyl-1H-indole-2-carbohydrazide;
N′-((4-iodophenyl)methylidene)-3-phenyl-1H-indole-2-carbohydrazide;
N′-((4-chlorophenyl)methylidene)-3-(4-(trifluoromethoxy)phenyl)-1H-indole-2-carbohydrazide;
N′-((4-bromophenyl)methylidene)-3-(4-(trifluoromethoxy)phenyl)-1H-indole-2-carbohydrazide;
N′-((4-bromophenyl)methylidene)-3-(3-chlorophenyl)-1H-indole-2-carbohydrazide;
N′-((4-bromophenyl)methylidene)-3-(4-(dimethylamino)phenyl)-1H-indole-2-carbohydrazide;
3-(2-chlorophenyl)-N′-((4-chlorophenyl)methylidene)-1H-indole-2-carbohydrazide;
N′-((4-bromophenyl)methylidene)-3-(2-chlorophenyl)-1H-indole-2-carbohydrazide;
4-((((3-phenyl-1H-indol-2-yl)carbonyl)hydrazono)methyl)benzenesulfonamide;
N′-((4-hydroxyphenyl)methylidene)-3-phenyl-1H-indole-2-carbohydrazide; and
4-((((3-phenyl-1H-indol-2-yl)carbonyl)hydrazono)methyl)phenyl sulfamate.

36. The compound of claim 30 wherein R² is selected from the group consisting of alkenyl, alkoxy, alkylsulfanyl, alkylsulfonyl, aminocarbonyl, arylalkyl, arylsulfanyl, arylsulfonyl, halo, and heterocycle.

37. The compound of claim 36 selected from the group consisting of
N′-((4-chlorophenyl)methylidene)-3-(phenylsulfonyl)-1H-indole-2-carbohydrazide;
N′-((4-bromophenyl)methylidene)-3-(phenylsulfonyl)-1H-indole-2-carbohydrazide;
3-benzyl-N′-((4-chlorophenyl)methylidene)-1H-indole-2-carbohydrazide;
3-benzyl-N′-((4-bromophenyl)methylidene)-1H-indole-2-carbohydrazide;
2-((2-(4-chlorobenzylidene)hydrazino)carbonyl)-N,N-diethyl-1H-indole-3-carboxamide;
2-((2-(4-chlorobenzylidene)hydrazino)carbonyl)-N,N-dimethyl-1H-indole-3-carboxamide;
2-((2-(4-chlorobenzylidene)hydrazino)carbonyl)-N-phenyl-1H-indole-3-carboxamide;
N′-((4-chlorophenyl)methylidene)-3-(methylthio)-1H-indole-2-carbohydrazide;
N′-((4-chlorophenyl)methylidene)-3-(methylsulfonyl)-1H-indole-2-carbohydrazide;
3-(methylsulfonyl)-N′-(1,3-thiazol-2-ylmethylidene)-1H-indole-2-carbohydrazide;
3-(methylsulfonyl)-N′-(2-naphthylmethylidene)-1H-indole-2-carbohydrazide;
N′-((4-chlorophenyl)methylidene)-3-methoxy-1H-indole-2-carbohydrazide;
3-bromo-N′-((4-chlorophenyl)methylidene)-1H-indole-2-carbohydrazide;
3-(methylsulfonyl)-N′-(1-naphthylmethylidene)-1H-indole-2-carbohydrazide;
N,N-dimethyl-2-((2-(2-naphthylmethylene)hydrazino)carbonyl)-1H-indole-3-carboxamide;
N′-((4-chlorophenyl)methylidene)-3-isopropoxy-1H-indole-2-carbohydrazide;
N′-((4-cyanophenyl)methylidene)-3-isopropoxy-1H-indole-2-carbohydrazide;
3-chloro-N′-(phenylmethylidene)-1H-indole-2-carbohydrazide;
3-chloro-N′-((4-chlorophenyl)methylidene)-1H-indole-2-carbohydrazide;
3-chloro-N′-((4-cyanophenyl)methylidene)-1H-indole-2-carbohydrazide;
3-chloro-N′-((4-(difluoromethoxy)phenyl)methylidene)-1H-indole-2-carbohydrazide;
N′-((4-chlorophenyl)methylidene)-3-thien-2-yl-1H-indole-2-carbohydrazide;
N′-((4-bromophenyl)methylidene)-3-thien-2-yl-1H-indole-2-carbohydrazide;
N′-((4-chlorophenyl)methylidene)-3-pyridin-4-yl-1H-indole-2-carbohydrazide;
N′-((4-bromophenyl)methylidene)-3-pyridin-4-yl-1H-indole-2-carbohydrazide;
N′-((4-bromophenyl)methylidene)-3-thien-3-yl-1H-indole-2-carbohydrazide; and
N′-((4-fluorophenyl)methylidene)-3-vinyl-1H-indole-2-carbohydrazide.

38. The compound of claim 30 wherein R¹ is halo.

39. The compound of claim 38 selected from the group consisting of
7-chloro-N′-((4-chlorophenyl)methylidene)-3-methyl-1H-indole-2-carbohydrazide;
7-chloro-N′-((4-methoxyphenyl)methylidene)-3-methyl-1H-indole-2-carbohydrazide;
N′-((4-bromophenyl)methylidene)-7-chloro-3-methyl-1H-indole-2-carbohydrazide;
7-chloro-N′-((4-chlorophenyl)methylidene)-3-phenyl-1H-indole-2-carbohydrazide;
7-chloro-N′-((4-cyanophenyl)methylidene)-3-phenyl-1H-indole-2-carbohydrazide;
N′-((4-chlorophenyl)methylidene)-7-fluoro-3-phenyl-1H-indole-2-carbohydrazide;
N′-((4-cyanophenyl)methylidene)-7-fluoro-3-phenyl-1H-indole-2-carbohydrazide;
N′-((4-bromophenyl)methylidene)-7-chloro-3-phenyl-1H-indole-2-carbohydrazide; and
N′-((4-bromophenyl)methylidene)-7-fluoro-3-phenyl-1H-indole-2-carbohydrazide.

40. A pharmaceutical composition comprising a compound of claim 22, or a therapeutically acceptable salt thereof, in combination with a therapeutically acceptable carrier.
